(19) United States
(12) Patent Application Publication
(10) Pub. No.: US 2017/0121678 A1
(43) Pub. Date: May 4, 2017

(54) METHODS AND COMPOSITIONS TO STABILIZE DIFFERENT STEM CELL STATES

(71) Applicants: UNIVERSITY OF WASHINGTON - CENTER COMMERCIALIZATION, Seattle, WA (US); SAGE BIONETWORKS, Seattle, WA (US)

(72) Inventors: Hannele Ruohola-Baker, Seattle, WA (US); Julie Mathieu, Seattle, WA (US); Henrik Sperber, Seattle, WA (US); Yuliang Wang, Seattle, WA (US); Jason Wayne Miklas, Seattle, WA (US)

(21) Appl. No.: 15/316,021

(22) PCT Filed: Jun. 2, 2015
(86) PCT No.: PCT/US15/33778

§ 371 Date: Dec. 2, 2016

Related U.S. Application Data
(60) Provisional application No. 62/006,774, filed on Jun. 2, 2014.

(51) Int. Cl. C12N 5/0735; C12Q 1/68; G01N 33/68

(52) U.S. Cl. CPC C12N 5/0606; G01N 33/6848; C12Q 1/6888; C12N 2501/235; C12N 2501/105; C12N 2501/115; C12N 2501/727

(57) ABSTRACT

Provided herein are methods and compositions for maintaining human stem cells in either a nave state or a primed state, inducing, promoting, inhibiting, or controlling the transition from one state to another, and detecting the state of a stem cell. Also disclosed are compositions comprising substantially homogenous populations of primed state or nave state stem cells.

### CROSS REFERENCE TO RELATED APPLICATIONS

This application claims priority to U.S. Provisional Patent Application 62/006,774 filed Jun. 2, 2014, which is incorporated by reference herein in its entirety.

### GOVERNMENT SUPPORT STATEMENT

This invention was made with U.S. government support under Grant No. GM083867, awarded by the National Institutes of Health. The government has certain rights in the invention.

### FIELD

The present disclosure relates to primed and naïve stem cells and methods and compositions for stabilizing the stem cells in either of the primed or naïve states.

### BACKGROUND

Pluripotent stem cells are able to self-renew and have the capacity to regenerate all tissues in the body. Unveiling the molecular mechanisms through which pluripotency is maintained holds tremendous promise for understanding early animal development and for developing therapies in regenerative medicine. Pluripotency does not represent a single defined state. Subtle states of pluripotency, with differences in measurable characteristics relating to gene expression, epigenetics and cellular phenotype, provide an experimental system for studying potential key regulators that constrain or expand the developmental capacity of pluripotent cells. Two stable pluripotent states which have been defined are preimplantation naïve and postimplantation primed states. Naïve, preimplantation human embryonic stem cells (hESCs) show higher developmental potential than postimplantation, primed hESCs.

Metabolic signatures are highly characteristic for a cell and are proposed to act as a leading cause for cell fate changes. Pluripotent stem cells have a unique metabolic pattern. The naïve to primed mouse ESC transition accompanies a dramatic metabolic switch from a bivalent to a highly glycolytic state. However, the primed state having inert mitochondria rapidly changes to highly respiring mitochondria during further differentiation.

In mouse embryonic stem cells (mESCs), threonine and S-adenosyl methionine (SAM) metabolism are coupled, resulting in regulation of histone methylation marks. Methionine and SAM are also required for the self renewal of hESCs, since depletion of SAM leads to reduced trimethylated histone H3K4 (H3K4me3) marks and defects in maintenance of the hESC state. SAM therefore is a key regulator for maintaining ESC undifferentiated state and regulating their differentiation. However, little is known about SAM levels or its regulation during the transition between naïve and primed human embryonic states. The epigenetic landscape changes from the naïve to primed state through increased H3K27me3 repressive methylation marks.

### SUMMARY

Provided herein are methods and compositions for maintaining human stem cells in either a naïve state or a primed state, inducing, promoting, inhibiting, or controlling the transition from one state to another, and detecting the state of a stem cell.

In certain embodiments, a method of detecting the developmental state of a stem cell is provided, the method comprising measuring the levels of one or more metabolites in a culture of the stem cells; and determining whether the stem cells are in a naïve or a primed state, wherein naïve state stem cells have higher levels of the one or more naïve state metabolites than the primed state stem cells, and primed state stem cells have higher levels of one or more primed state metabolites than the naïve state stem cells. In some embodiments, the one or more metabolites are primed state metabolites or naïve state metabolites.

In other embodiments, a method of promoting the transition of stem cells from a naïve state to a primed state is provided, comprising: culturing the stem cell in a culture medium supplemented with at least one primed state metabolite; and determining the level of kynurenine in the stem cells, wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In yet other embodiments, a method of promoting the transition of stem cells from a primed state to a naïve state is provided, comprising: culturing the stem cell in a culture medium supplemented with at least one naïve state metabolite and determining the level of kynurenine in the stem cells, wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In some embodiments, a method of maintaining stem cells in a naïve state is provided comprising culturing naïve state stem cells in a culture medium supplemented with at least one naïve state metabolite. In some embodiments, the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In other embodiments, a method of maintaining stem cells in a primed state is provided comprising culturing primed state stem cells in a culture medium supplemented with at least one primed state metabolite. In other embodiments, the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In yet other embodiments, a method of inhibiting the transition of stem cells from a primed state to a naïve state is provided, comprising culturing primed state stem cells in a culture medium supplemented with at least one primed state metabolite. In other embodiments, the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In some embodiments, a method of inhibiting the transition of stem cells from a naïve state to a primed state is provided comprising culturing naïve state stem cells in a culture medium containing at least one naïve state metabolite. In other embodiments, the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In other embodiments of the methods provided herein, the stem cell is an embryonic stem cell, a germline stem cell, an induced pluripotent stem cell, an adult stem cell, a hematopoietic stem cell, and a dental pulp stem cell.

In other embodiments of the methods provided herein, the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

In yet other embodiments of the methods provided herein, the stem cells are cultured with the primed state metabolite or the naïve state metabolite for at least three days to reach the desired state.

In some embodiments of the methods provided herein, the primed state metabolite is a breakdown product of tryptophan, S-adenosyl methionine (SAM), succinate, fructose (1,6/2,6)-biphosphonate, lactate, methionine, nicotinamide, kynurenine, long carbon chain lipids, or an aryl hydrocarbon receptor (AHR) ligand, or an inducer of indoleamine 2,3-diozygenase 1 (IDO1), IDO2, or tryptophan 2,3-dioxygenase 2 (TDO2), or an inhibitor of nicotinamide-N-methyl-transferase (NNMT).

In other embodiments of the methods provided herein, the naïve state metabolite is a glycogen synthase kinase 3 (GSK3) inhibitor, a mitogen-activated protein kinase (MEK) inhibitor, 1-methylnicotinamide (1-MNA), tryptophan, S-adenosylhomocysteine (SAH), or an inhibitor of indoleamine 2,3-diozygenase 1 (IDO1), IDO2, or tryptophan 2,3-dioxygenase 2 (TDO2), or an inducer of nicotinamide-N-methyl-transferase (NNMT).

In yet other embodiments of the methods provided herein, the AHR ligand is a halogenated aromatic hydrocarbon such as a polychlorinated dibenzodioxin (2,3,7,8-tetrachlorodibenzo-p-dioxin (TCDD), a dibenzofuran, or a biphenyl, a polycyclic aromatic hydrocarbon such as (3-methylcholanthrene, a benzo(a)pyrene, a benzanthracene, or a benzoflavone, a derivative of tryptophan such as an indigo dye or indirubin, a tetrapyrrole such as bilirubin, an arachidonic acid metabolite such as lipoxin A4 or prostaglandin G, a modified low-density lipoprotein, or a dietary carotenoid.

In other embodiments of the methods provided herein, the long carbon chain lipid has a carbon chain length between about 25 and about 50 carbons, or between about 35 and about 45 carbons, or longer than about 40 carbons.

In other embodiments of the methods provided herein, the MEK inhibitor is trametinib, selumetinib, binimetinib, PD-325901, cobimetinib, CI1040, or PD035901.

In some embodiments, compositions are provided comprising naïve state stem cells produced by the methods disclosed herein.

In some other embodiments, compositions are provided comprising primed state stem cells produced by the methods disclosed herein.

In yet other embodiments, a method of treating a disorder in a subject in need thereof is provided, the method comprising administering to the subject a composition of primed or naïve state stem cells produced by the methods disclosed herein. In certain embodiments, the disorder is diabetes, cardiovascular disease, neurodegenerative diseases, spinal cord injury, brain injury, various aspects of aging, wound healing, and dental disorders.

### BRIEF DESCRIPTION OF THE DRAWINGS

FIGS. 1A-1M depict that naïve and primed ESCs are metabolically different. FIG. 1A: PCA of RNA-seq and microarray data from this study (Elf1, H1, in vivo mouse ICM, in vivo mouse Epiblast), Chan 2013. (Chen et al., Cell Stem Cell 13:663-675, 2013), Gafni 2013 (Gafni et al., Nature 504:282-286, 2013), Theunissen 2014 (Theunissen et al. Cell Stem Cell 15:471-487, 2014), Takashima 2014 (Takashima et al., Cell 158:1254-1269, 2014) and single-cell RNA-seq data from Yan 2014 (Yan et al., Nat. Struct. Mol. Bio. 20:1131-1139, 2013). FIG. 1B: Metabolic profile of naïve and primed human pluripotent stem cells (naïve: Elf1 and H1 4iLIF; primed: H1). A representative trace of oxygen consumption rate (OCR) change is shown under a MitoStress protocol (sequential addition of oligomycin, carbonyl cyanide p-(trifluoromethoxy)phenylhydrazone [FCCP], and antimycin/rotenone). FIG. 1C: Primed hESCs (H7 and H1) have reduced OCR changes in response to FCCP following oligomycin treatment compared to naïve hESCs (Elf1 and H1 4iLIF). n=3, SEM: **p<0.01, ***p<0.001, 2-tailed t-test. FIGS. 1D and 1E: Transition of naïve hESCs Elf1 (FIG. 1D) and WIN1 (FIG. 1E) toward a more primed state by culture in activin A/FGF (AF) media reduced OCR changes in response to FCCP after 1-3 days (n=3, SEM **p<0.01, ***p<0.001, 2-tailed t-test). FIGS. 1F-1G: Naïve hESCs (Elf1) and primed hESCs (Elf1 AF) have similar mitochondrial DNA copy number (FIG. 1F) and mitochondrial mutation frequencies (FIG. 1G). FIG. 1H: log 2 fold expression change of all cytochrome C oxidases (COX) genes between primed and naïve states are shown in both our data set (left) and Teunissen 2014 (right). Consistently, COX genes are downregulated in the primed state. FIG. 1I: Hypoxia inducible domain family, member 1A (HIGD1A) is consistently downregulated in primed hESCs vs naïve hESCs in our study and others. FIG. 1J: Hypoxia inducible factor 1, alpha subunit (HIF1a) protein is stabilized in primed hESCs (H7 and Elf1 AF). FIG. 1K: Proteomic workflow used to identify differentially regulated protein expression in primed vs. naïve hESCs. FIG. 1L: Volcano plot of differentially expressed proteins in primed hESCs (right, green; Elf1 AF) vs naïve cells (left, blue, Elf1). Significant hits are shown (FDR<0.05). Proteins were quantified by nano-LC-MS/MS on a Fusion Orbitrap. FIG. 1M: JARID2 (Jumonji AT rich interactive domain 2) and lactate dehydrogenase (LDHA) proteins are upregulated in primed hESCs (Elf1 AF and H7) compared to naïve hESCs (Elf1), as revealed by Western blot analysis.

FIGS. 2A-2I depict metabolomic analysis of naïve and primed ESCs. FIG. 2A: Scheme of mass spectrometry experiments performed for metabolites on mouse and human naïve (pre-implantation) and primed (post-implantation) ESCs. FIGS. 2B-2C depict naïve and primed stem cells can be clearly separated based on their metabolic profiles. FIG. 2B: A principal component analysis (PCA) plot of water-soluble untargeted GC-MS metabolomics data. The first principal component (PC), which separates the primed cell types (left) from the naïve cell types (right) explained 50.5% of total variance. FIG. 2C: PCA plot of untargeted LC metabolomics data. Three clusters are along the first PC: primed cells (left), primed cells toggled back to naïve cells (middle) and naïve cells (right). The first PC explained 68.2% of total variance. FIGS. 2D-2G depict volcano plots of differentially abundant metabolites between primed and naïve cells in mESCs (FIGS. 2D and 2E) and hESCs (FIGS. 2F and 2G). x-axis is log 2 fold change of abundance, y-axis is negative log 10 of p-value. Metabolites of biological interest for further analysis are labeled. FIGS. 2H and 2I: Fold change of glycolysis metabolites (n=3, **p<0.01, 2-tailed t-test) after log 2 transformation and mean centering in H 1 vs. Elf1 detected by targeted LC-QQQ-MS.

FIGS. 3A-3J depict primed ESCs accumulate lipids while naïve ESCs use fatty acids as a source of energy. FIGS. 3A-3B: More abundant lipids in primed cells (H1) have more carbon atoms (FIG. 3A) and larger mass (FIG. 3B) than more abundant lipids in naïve cells (Elf1). FIG. 3C: More abundant lipids in primed cells (R1AF) are more unsaturated than more abundant lipids in naïve cells (R1). FIG. 3D: Carnitine palmitoyltransferase 1A (CPT1A) is downregulated in human and mouse primed ESCs compared to naïve ESCs in our study and others (relative fold change of CPT1A expression, RNA seq or microarray). FIG. 3E: ChIP-seq analysis of CPT1A gene shows more repressive H3K27me3 marks and less active H3K4me3 and H3K27ac marks in primed hESCs (C1, WIBR3 5, H1, H9 48) than naïve hESCs (Elf18; naïve CI, naïve BGO1, naïve WIBR35). FIG. 3F: volcano plot representation of microRNAs expression in naïve hESCs (Elf1) and primed hESCs (H1, ENCODE). FIG. 3G: qPCR expression of microRNA miR-9 (predicted to target CPT1A) and miR-10a (predicted to target FASN) showing a 34-fold higher expression of hsa-miR-10a in Elf1 compared to H1, and 4-fold higher expression of hsa-miR-9 in H1 compared to Elf1 (n=3, SEM, *p<0.05, **p<0.01, 2-tailed t-test). FIGS. 3H-3J: Seahorse palmitate assay shows that naïve human and mouse ESCs use fatty acids as a source of energy. A representative trace of OCR changes is shown after addition of two doses of palmitate or BSA vehicle followed by two doses of ETO (etomoxir) in human ESCs (naïve Elf1 and primed H7, FIG. 3H) and mouse ESCs (naïve R1 and primed EpiSCs, FIG. 3I). Changes after ETO injections were quantified in FIG. 3J (n=3, SEM *p<0.05, **p<0.01, 2-tailed t-test).

FIGS. 4A-4M depict that amino acids methionine and tryptophan are differentially regulated in naïve and primed hESCs. FIG. 4A: Model of the tryptophan-kynurenine pathway. FIG. 4B: IDO1 is highly expressed in primed hESCs (H1, H7, Elf1 AF) as compared to naïve hESCs (Elf1 2iLIF, H1 2iF, H1 4iLIF), as shown by qPCR analysis (n=3, SEM, ***p<0.001, 2-tailed t-test). FIG. 4C: The kynurenine vs. tryptophan ratio is higher in primed hESCs (H1, H7, Elf1 AF) than naïve hESCs (Elf1, WIN1, H1 2iF), as detected in three independent targeted mass spectrometry experiments and four non targeted mass spectrometry experiments. (SEM, *p<0.05, **p<0.01, ***p<0.001, 1-tailed t-test, non-targeted n=6, HILIC targeted n=4, QQQ n=3). FIG. 4D: Kynurenine is secreted in the media by primed hESCs (H7). n=5, SEM *p<0.05, 2-tailed t-test. FIG. 4E: Addition of kynurenine (100 μM, 2 days) reduced OCR changes in response to FCCP in naïve hESCs (WIN1), SEM ***p<0.001, 2-tailed t-test. FIG. 4F: Model of S-adenosyl-L-methionine (SAM) pathway and nicotinamide N-methyltransferase (NNMT). Metabolites in red are up-regulated in primed hESCs while metabolites and enzymes in blue are up-regulated in naïve hESCs. FIG. 4G: Volcano plot of RNA-seq data from naïve hESCs (Elf1) and primed hESCs (H1). Genes with greater than 2 fold change and false discovery rate <0.05 are colored. NNMT and IDO1 are among the most differentially expressed genes. FIG. 4H: NNMT is highly up-regulated in naïve hESCs (H7 5iLIF, WIN1 5iLAF, H7 5iLAF, WIN1 5iLA, H1 2iF, Elf1 2iLIF, H1 4iLIF, H1 4iLTF) compared to primed hESCs (WIN1 F, H1, H7, Elf1 AF), as shown with qPCR analysis. Numbers indicate fold changes of naïve hESCs compared to H1 and H7 primed hESCs. (SEM, ***p<0.001, 2-tailed t-test, n=3) FIG. 4I: Naïve hESCs (WIN1, Elf1, H1 2iF, H1 4iLIF) have higher amounts of the NNMT product, 1-methylnicotinamide (1MNA), than primed hESCs (H1). (SEM **p<0.01, ***p<0.001, 2-tailed t-test) FIG. 4J: SAM levels are higher in primed hESCs (H1) than in naïve hESCs (Elf1), while S-adenosyl-L-homocysteine (SAH) levels are lower in naïve hESCs (Elf1) than primed hESCs (H1). (SEM, *p<0.05, **p<0.01, 1-tailed t-test, n=4) FIGS. 4K4-L: SAM induces a “primed-like” metabolic profile in naïve hESCs. Addition of SAM (500 μM) for 5 hr in media without methionine reduces OCR changes in response to FCCP in naïve hESCs (WIN1). n=4, SEM *p<0.05, 2-tailed t-test. FIG. 4M: Overexpression of NNMT delays the metabolic transition from naïve to primed. Cells transfected with NNMT over expression (OE) construct have increased OCR changes in response to FCCP compared to cells transfected with a catalytically inactive NNMT mutant (Y20A) in naïve hESCs transitioning to primed hESCs (Elf1 AF, 2 days). SEM *p<0.05, 2-tailed t-test.

FIGS. 5A-5R depicts high NNMT expression in naïve hESCs regulates histone methylation status. FIGS. 5A-5B: H3K27me3 reads mapped 5 kb around transcription start sites (TSS) of 648 developmental genes were plotted for Ware 2014 (Ware et al., Proc. Natl. Acad. Sci. USA 111:4484-4489, 2014), Gafni 2013, Theunissen 2014, Bernstein 2010 (Bernstein et al., Nat. Biotechnol. 28:1045-1048, 2010) (FIG. 5A), as well as Chan 2013 (FIG. 5B) ChIP-seq data sets. All showed that primed cells have more H3K27me3 repressive marks around TSS. Cr Western blot analysis show higher H3K27me3 and H3K9me3 in primed hESCs (H7) than naïve hESCs (Elf1) while H3K9/K14Ac does not change. FIG. 5D: qPCR analysis shows a knock-down regulation of NNMT using siRNA (50 nM, 72 hr) in naïve hESCs (Elf1), inducing a decrease of 1MNA levels and a downregulation of miR-10a. (SEM, *p<0.05, ***p<0.001, 2-tailed t-test, HILIC n=4, qPCR n=3) FIG. 5E: Western blot analysis of histone marks in Elf1 cells treated with siRNA against NNMT or siRNA against luciferase as a control. FIG. 5F: Western blot analysis of histone modifications after treatment of Elf1 cells with 100 μM of STAT3 inhibitor. FIG. 5G: Six hour treatment with STAT3 inhibitor (100 μM) in Elf1 cells increases H3K27me3 marks, as shown by ChipSeq analysis on all genes. FIG. 5H: Wnt ligands HIGD1A and EGLN1 are among the 313 overlapping genes with increased H3K27me3 mark in primed vs. naïve hESCs (Gafni 2013, Theunissen 2014, Bernstein 2010), and Elf1 cells treated for 6 hr with 100 μM STAT3 inhibitor vs. Elf1 cells. FIG. 5I: Windowed chromatin heatmaps of H3K27me3 profile±5 kb of promoters of the 313 overlapping genes with increased H3K27me3 FIG. 5J: Wnt is activated in naïve hESCs. Endogenous Wnt signaling in naïve (Elf1) and primed (Elf1 AF) BAR-reporter cells. Scale bars represent 200 μm. FIG. 5K: Wnt inhibitor IWP2 (2 μM) and Wnt antagonist XAV939 (5 μM) inhibit the reporter activity in naïve Elf1 cells after 72 h. Scale bars represent 200 μm. FIG. 5L: Wnt inhibition by IWP2 (2 μM, 72 hr) downregulates NNMT and miR-372 expression in naïve hESCs (Elf1) as shown by qPCR analysis. (SEM, *p<0.05, ***p<0.001, 2-tailed t-test, n=3) FIG. 5M: Wnt inhibition by IWP2 (2 μM, 48 hr) decreases OCR changes after FCCP in naïve hESCs (Elf1, WIN1) and in naïve hESCs transitioning to primed (WIN1 AF), SEM **p<0.01, ***p<0.001, 2-tailed t-test. FIG. 5N. Model of self-reinforcing loop between WNT and NNMT in naïve hESCs. FIG. 5O: screen shot of RNA expression and H3K27me3 marks of EGLN1 (PHD2) in naïve hESCs [Elf1 (Ware 2014), WIRB3 naïve and BGO1 naïve (Gafni 2013)], primed hESCs [WIRB3 primed (Gafni 2013), H1 and H9 (Bernstein 2010) and Elf1 treated with STAT3 inhibitor (100 NM) for 6 hr. FIG. 5P: HIFa is hydroxylated on prolyl residues by EGLN1 (PHD2), leading to VHL-mediated proteolysis. FIG. 5Q: HIF1a overexpression increases H3K27me3 marks. EV=empty vector control. FIG. 5R: Model of the intricate relationship between metabolism and epigenetic in hESCs. Primed hESCs have downregulated NNMT expression compared to naïve hESCs, resulting in increased levels of SAM and induction of H3K27me3 repressive marks on genes involved in metabolism switch.

FIGS. 6A-6I. FIG. 6A: PCA of RNA-seq and microarray data from this study, Chan 2013, Gafni 2013, Theunissen 2014, Takashima 2014 and Yan 2013. Expression values of H1 and H12i samples were normalized to the mean expression level of H12i samples; expression values of ELFAF and ELFAF were normalized to the mean expression level of ELF. FIG. 6B: PCA of microarray data (Elf1, H12iF, Elf1AF, H1; Ware 2014). FIGS. 6C-6D: Representative trace of OCR changes during the transition from naïve hESCs (Elf1, C and WIN1, FIG. 6D) to primed hESCs (cultured in activin A and FGF, overnight or for 2 or 3 days). FIG. 6E: ECAR changes after oligomycin injection in naïve hESC (Elf1, H1 4iLIF) and primed hESCs (H1, H7). FIG. 6F: ECAR changes after oligomycin injection during the transition from naïve hESCs (Elf1) to primed hESCs (Elf1 AF overnight, 2 or 3 days). FIG. 6G: Representative trace of OCR changes in primed hESCs (H7) and naïve hESCs (Elf1). FIGS. 6H-6I: Metabolic profile of primed hESCs (H1), hESCs toggled toward a more naïve state (H1 2iF) and naïve mouse embryonic stem cells (R1). A representative trace of OCR changes is shown (FIG. 6H). Primed hESCs (H1) have reduced OCR changes in response to FCCP following glucose treatment compared to naïve human and mouse ESCs (H1 2iF and R1, respectively; FIG. 6I).

FIGS. 7A-7E. FIGS. 7A-7B: Mitochondrial DNA copy numbers quantified in naïve hESCs (Elf1) and primed hESCs (H1, H7). FIG. 7C: Mitochondrial DNA mutation frequency in naïve hESCs (Elf1) and primed hESCs (H1). FIG. 7D: Mitochondrial DNA deletion frequency in naïve hESCs (Elf1) and primed hESCs (H1) FIG. 7E: Label-free quantification of protein expression is reproducible. Tryptic digestions of naïve hESCs (Elf1 2iLIF) or primed hESCs (Elf1 AF) were analyzed in triplicate by nano-LC MS/MS, with an average Pearson correlation of 0.86.

FIGS. 8A-8G. Log 2 fold expression change of mitochondria complexes genes between primed and naïve stages are shown in our data set (H1 vs. Elf1, FIG. 8A), Grow 2015 (Grow et al., Nature 2015 Apr. 20, doi: 10.1038/nature14308) (Elf1 AF vs. Elf1, FIG. 8B), Takashima 2014 (FIG. 8C), Theunissen 2014 (FIG. 8D), Gafni 2013 (FIG. 8E), Chan 2013 (FIG. 8F) and Tesar 2007 (Tesar et al., Nature 448:196-199, 2007) (FIG. 8G).

FIGS. 9A-9D. FIG. 9A: Heatmap generated through hierarchical Pearson clustering of metabolite expression from GC-TOF shows that naïve ESCs cluster away from primed ESCs, regardless of origin (mouse or human). FIG. 9B: volcano plot of differentially abundant metabolites between primed hESCs (Elf1 AF) and naïve hESCs (Elf1). FIGS. 9C-9D: PCA plot of water-soluble untargeted GC-MS (FIG. 9C) and LC-MS (FIG. 9D) metabolomics data showing a separation of naïve ESCs (mouse:R1 and human: Elf1) and primed ESCs (mouse: Epi, and human: Elf1 AF, H1, H7).

FIGS. 10A-10I. FIGS. 10A-10B: BODIPY 493/503 staining shows an increase of lipid droplet accumulation in primed hESCs (H7, H1, Elf1 AF) compared to naïve hESCs (Elf1, H1 2iF, H1 4iLIF). Images were taken at 5× magnification (FIG. 10A) and 20× magnification (FIG. 10B). FIG. 10C: BODIPY 493/503 staining shows an increase of lipid droplet accumulation in primed mESCs (EpiSCs) compared to naïve mESCs (R1). FIG. 10D: Oil Red 0 staining shows an increase of lipid droplet accumulation in primed hESCs (H7) compared to naïve hESCs (Elf1). FIG. 10E: H3K27me3 reads mapped 5 kb around transcription start site (TSS) of CPT1A were plotted for Chan 2013. ChIP-seq data sets. Primed cells have more H3K27me3 repressive marks around TSS of CPT1A. FIG. 10F: Expression of key genes involved in fatty acid synthesis from RNA-seq analysis in naïve (Elf1) and primed (H1) hESCs. FIG. 10G: Representative trace of OCR changes under Seahorse palmitate assay with addition of 2 doses of palmitate or BSA vehicle followed by 2 doses of ETO in hESCs H1 pushed toward a more naïve state using 4iLIF (H1 4iLIF) and primed hESCs H1. FIG. 10H: More abundant lipids in primed human cells (Elf1 AF) are more unsaturated than more abundant lipids in naïve human cells (Elf1). FIG. 10I: More abundant lipids in primed mouse cells (EpiSC) have more carbon atoms than more abundant lipids in naïve mouse cells (R1).

FIGS. 11A-11B. depicts the relative fold change of expression of genes involved in transport of fatty acids into mitochondria and genes involved in 4 steps of fatty acid beta-oxidation from RNASeq analysis in human ESCs (H1 vs. Elf1, FIG. 11A) and mouse ESCs (Epiblasts vs.ICM, FIG. 11B).

FIGS. 12A-12C. FIG. 12A: RNA expression of IDO1 in human 8-cell embryo and primed hESCs at passage 0 (hESCpO) and passage 10 (hESCp10) analyzed by single cell RNA Seq data. FIG. 12B: RNA expression of IDO1, IDO2, tryptophan 2,3-dioxygenase (TDO2) and aminoadipate aminotransferase (AADAT) in naïve hESCs Elf1, primed hESCs H1 and H1 differentiated during 4 days detected by microarray. FIG. 12C: IDO expression in H1 cells and H1 differentiated toward various lineages.

FIGS. 13A-13C. FIG. 13A: NNMT expression in various tissues analyzed by RNASeq. FIG. 13B: NNMT expression in various organs of rats over time (from 2 to 104 weeks) analyzed by RNASeq. FIG. 13C: NNMT expression in H1 cells and H1 differentiated toward various lineages (Chadwick, 2012).

FIGS. 14A-14J. FIGS. 14A-14D: Reads for H3K4me1 (FIG. 14A), H3K4me3 (FIG. 14B), H3K9me3 (FIG. 14C) and H3K27ac (FIG. 14D) mapped 5 kb around transcription start sites (TSS) were plotted for Gafni 2013. ChIP-seq data set. FIG. 14E-14F: Reads for H3K4me3 (FIG. 14E) and H3K27ac (FIG. 14F) mapped 5 kb around TSS were plotted for Chan 2013 ChIP-seq data set. FIG. 14G: Reads for H3K4me3 mapped 5 kb around TSS were plotted for Theunissen 2014 ChIP-seq data set. FIG. 14H: qPCR analysis of miR-518b and miR-520f in Elf1 cells after transfection with siRNA against NNMT or luciferase (50 nM, 72 hr). FIG. 14I: Western blot analysis of H3K4me3 mark in naïve hESCs (Elf1) transfected or not with siRNA against NNMT (50 nM, 72 hr) and in primed hESCs (Elf1 AF). FIG. 14J: 1MNA (0.5 mM, 72 hr) reduces H3K27me3 mark in primed hESCs (Elf1 AF).

FIGS. 15A-15B. RNA-seq data of histone methyltransferases (FIG. 15A) and histone demethylases (FIG. 15B) in naïve (Elf1) and primed (H1) hESCs.

FIGS. 16A-16C. FIG. 16A: STAT3 is phosphorylated in H1 cells pushed toward a more naïve stage (H1 2iF), even without LIF addition to the media. FIG. 16B: qPCR analysis of NNMT expression after treatment of Elf1 cells with 100 μM of STAT3 inhibitor. FIG. 16C: H3K27me3 reads from ChIP-seq data mapped 5 kb around transcription start sites (TSS) were plotted for naïve hESCs (C1, WIBR3, BGO1 from Gafni 2013, and Elf1 from Ware 2014), primed hESCs (C1, WIBR3 from Gafni 2013, H1 from Ware 2014), and naïve hESCs Elf1 treated for 6 h with 100 μM of STAT3 inhibitor. STAT3 inhibitor treatment increases H3K27me3 on 313 overlapping genes between primed vs. naïve hESCs (Gafni 2013) and STAT3i vs. Elf1.

FIGS. 17A-F depict upregulation of Wnt ligands and Wnt targets in naïve hESCs compared to primed hESCs, as detected by RNA seq in this study (FIG. 17A), Grow 2015 (FIG. 17B), Chan 2013 (FIG. 17C), Takashima 2014 (FIG. 17D) and microarray in Gafni 2013 (FIG. 17E) and Theunissen 2014 (FIG. 17F).

FIG. 18A-18D. FIG. 18A: siRNA against R-catenin inhibits the reporter activity in naïve Elf1 cells after 72 hr. Scale bars represent 200 μm. FIG. 18A: BAR reporter is activated in naïve hESCs (Elf1 2iL). FIG. 18C: treatment of primed hESCs (Elf1 AF) with Wnt3a CM or GSK3 inhibitor (CHIR99021, 10 μM) for 3 days induces differentiation and re-activation of the BAR reporter. FIG. 18D: Seahorse representative trace of OCR following mitostress protocol in naïve hESCs (WIN1) with or without treatment with Wnt inhibitor IWP2 (2 μM, 48 hr).

FIG. 19A-19B depict expression of NNMT (FIG. 19A) and IDO1 (FIG. 19B) in naïve hESCs (Elf1) after infection with an empty vector (EV) or HIF1 overexpressing (HIF1 OE) virus, analyzed by qPCR.

FIG. 20A-20B. FIG. 20A: qPCR analysis of miR-200b in naïve hESCs (Elf1) and primed hESCs (H1). FIG. 20B: details of the model presented in FIG. 5N showing some of the genes with H3K27me3 mark found in the overlap between primed hESCs and naïve hESCs treated with STAT3 inhibitor, and their possible involvement in the metabolic switch occurring between naïve and primed hESCs. Shown are miRNAs predicted to regulate those genes and that have a change in expression inversely correlating with the expression of those genes.

### DETAILED DESCRIPTION

Provided herein are methods and compositions for maintaining human stem cells in either a naïve state or a primed state, inducing, promoting, inhibiting, or controlling the transition from one state to another, and detecting the state of a stem cell.

Naïve stem cells have more robust developmental potential than primed stem cells. Metabolic signatures are highly characteristic for a cell and may act as a leading indicator for cell fate changes, preceding changes in cell fate genes. During the transition from the naïve to the primed state, the cells undergo a dramatic transition from metabolically bivalent to highly glycolytic. However, the primed state of inert mitochondria rapidly changes to highly potent mitochondria during further differentiation. It is not yet understood how and why the pluripotent cells enter the highly glycolytic, metabolically cancer-like (Warburg effect) state and how a differentiating cell leaves this stage.

Metabolomic profiling of naïve and primed human endothelial stem cells (hESCs) shows a difference in metabolite profile between naïve and primed cells, which is consistent across species for human and mouse. Glucose levels are reduced and lactate levels increase in the primed state, which is consistent with the cells becoming exclusively glycolytic. Further study of the glycolysis shows a high upregulation of fructose 1,6-bisphosphate (F16BP) due to upregulation of phosphofructokinase (PFK) and downregulation of gluconeogenesis gene fbp (fructose 1,6-bisphosphate) in primed ESCs indicating that the accumulation of F16BP is due to upregulated glycolysis and downregulated gluconeogenesis.

Interestingly glyceraldehyde-3-phosphate (G3P), the downstream metabolite of F16BP, does not increase in primed stem cells. G3P can be conserved for biosynthetic purposes in the synthesis of fatty acids and amino acids and a significant increase in long carbon chain lipids is seen in primed stem cells, an indication of increased synthesis and/or decreased beta-oxidation.

In addition primed cells show high enrichment of the tryptophan degradation product kynurenine. Kynurenine can act as a ligand for the transcription factor AHR (aryl hydrocarbon receptor). In cancer cells, AHR activation by kynurenine is shown to induce growth while in surrounding T-cells, kynurenine-based AHR activation inhibits the immune response against cancer cells. Microarray and qPCR data showed a high increase of the tryptophan degrading enzyme indoleamine-pyrrole 2,3-dioxygenase 1 (IDO1) in primed hESC, which, in combination with the increase in glycolytic products, explains the mechanism by which kynurenine is accumulated. After peaking in primed ESCs, IDO1 levels quickly drop as the ESCs begin to differentiate, indicating that the function of IDO1 is specific for the primed stage. IDO1 levels are 60-fold higher in primed hESC (H1) compared to naïve hESC (Elf1). Kynurenine is a key metabolite acting in primed hESC in a manner similar to cancer cells; in primed hESC kynurenine may support stem cell growth and self-renewal when secreted from hESC. Additionally, kynurenine may inhibit Treg cell proliferation, thereby providing protection to the primed stage embryo by silencing the mother's immune cells through AHR activation.

Increased trimyethylated histone H3K27 (H3K27me3) repressive methylation mark expression during naïve to primed human embryonic stem cell (hESC) transition is regulated by cellular metabolite and nicotinamide-N-methyl-transferase (NNMT) levels. The epigenetic repressive mark regulates the hypoxia inducing factor (HIF) and Wnt pathways and electron transport chain supercomplex stabilizer, thereby controlling the key metabolic switch observed during naïve to primed pluripotent transition. Increased lipid biosynthesis, reduced beta-oxidation, and reduced mitochondrial oxygen consumption is seen in primed hESCs. Metabolic and gene expression analysis revealed that NNMT and its enzymatic product 1-methylnicotinamide (1-MNA) are highly upregulated, while the substrates nicotinamide and SAM are downregulated in the naïve state, correlating with reduced H3K27me3 marks. Furthermore, increased SAM levels accelerate, while NNMT overexpression represses the naïve to primed hESC metabolic transition. Knockdown of NNMT in naïve hESCs increases H3K27me3 repressive marks in developmental as well as key metabolic genes that regulate the metabolic switch in naïve to primed transition. NNMT consumes SAM in naïve cells, making it unavailable for histone methylation. Histone methylation further regulates the key signaling pathways important for the metabolic changes that are necessary for early human development.

Human naïve and primed stem cells display distinct metabolic profiles. Switching between these metabolic states is regulated by NNMT, which controls the amount of SAM available for polycomb repressive complex 2 (PRC2)-dependent H3K27me3 histone methylation. Repressive histone methylation then controls the primed hESC specific metabolism through the Wnt and HIF pathways. The naïve to primed stem cells transition shows a reduction in Wnt signaling, electron transport chain activity, and fatty acid beta-oxidation and increase in mechanisms involved in lipid biosynthesis and HIF1a stabilization. In naïve stem cells, NNMT, and its enzymatic product 1-MNA, are highly upregulated, while the substrates nicotinamide and SAM are downregulated, correlating with reduced H3K27me3 marks. Inhibition of the NNMT regulator signal transducer and activator of transcription 3 (STAT3) in naïve hESCs increases H3K27me3 repressive marks in developmental and metabolic genes, including Wnt signaling and the HIF1 repressor, prolyl hydroxylase EGLN1 (egl-9 family hypoxia-inducible factor 1) as well as HIGD1, a key regulator of electron transport chain super complex formation. NNMT consumes SAM in naïve cells, making it unavailable for histone methylation which represses Wnt pathway and electron transport chain activity and activate HIF pathway and lipid synthesis, facilitating the metabolic switch in the naïve to primed hESC transition. Differential metabolites between pluripotent states control epigenetic dynamics and signaling.

Primed stem cells are dependent on glycolysis while early glycolysis metabolites are upregulated, the downstream metabolites are downregulated in primed state stem cells, suggesting that metabolites are being channeled off to increase the amount of glycerol backbone available for biosynthesis of lipids in primed cells, or for the one-carbon cycle for methylation reactions by SAM. SAM can also be regulated by NNMT, which is dramatically downregulated in primed compared to naïve hESCs, making SAM available as a substrate for DNA and histone methylation. A difference in SAM levels between naïve and primed hESCs correlates with dramatic changes in H3K27me3 marks and reveal NNMT as a key regulator of these changes. While SAM-dependent regulation of histone methylation is present in stable primed hESCs, the effect was mainly observed in trimethylated histone H3K4 (H3K4me3), not in H3K27me3 marks. It is plausible that H3K27me3 marks, once established are less dynamic in primed hESCs than H3K4me3 marks.

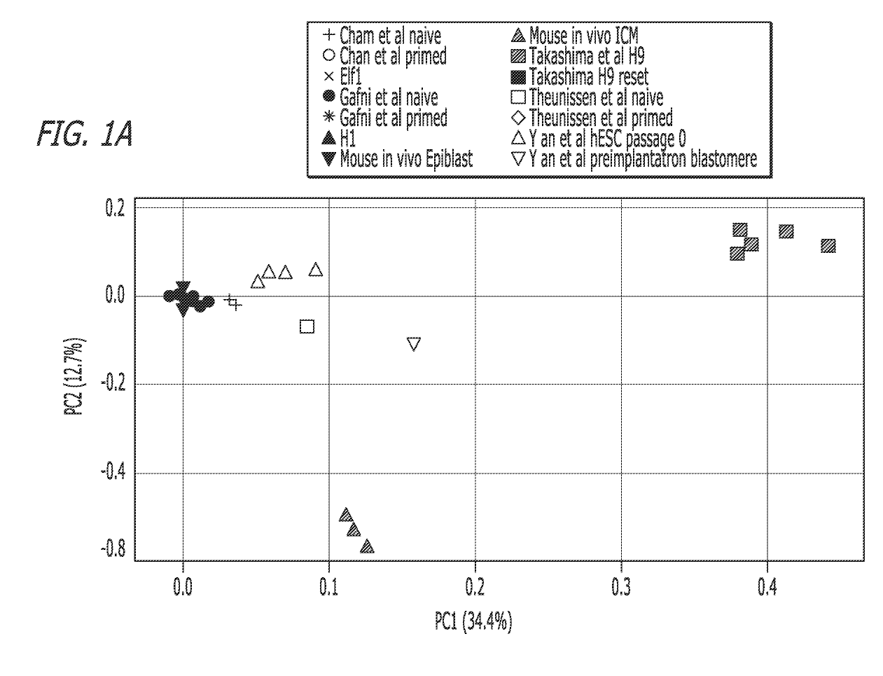

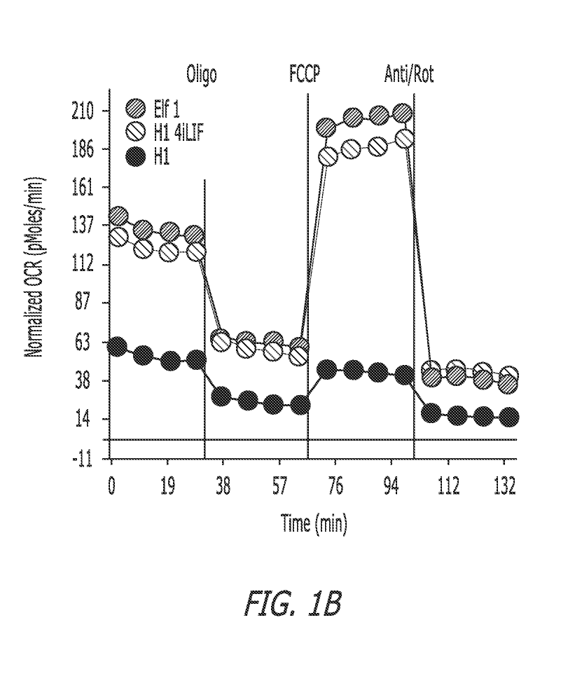

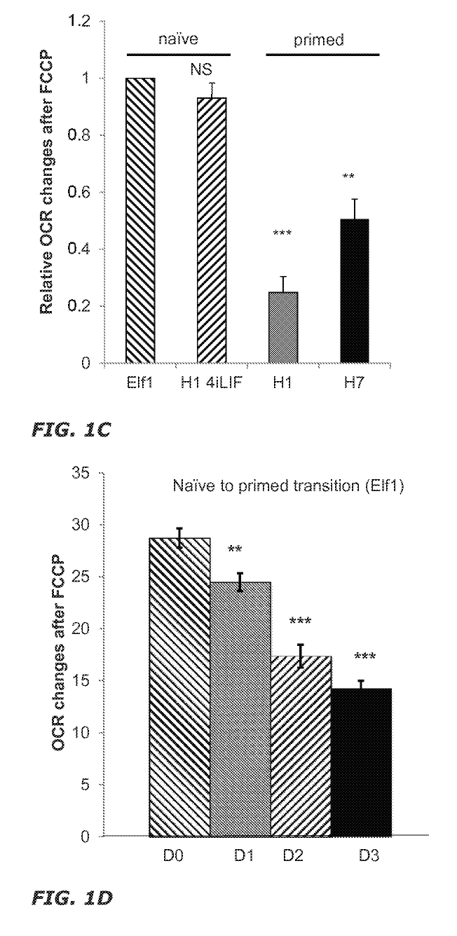

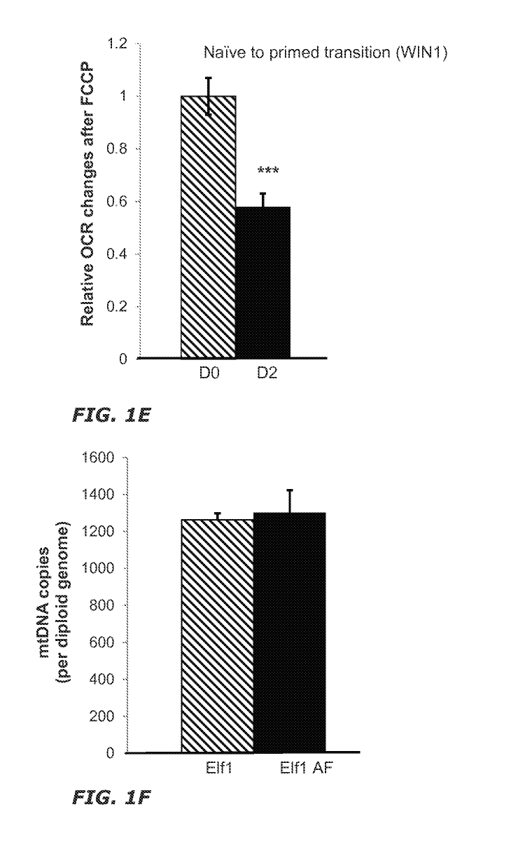

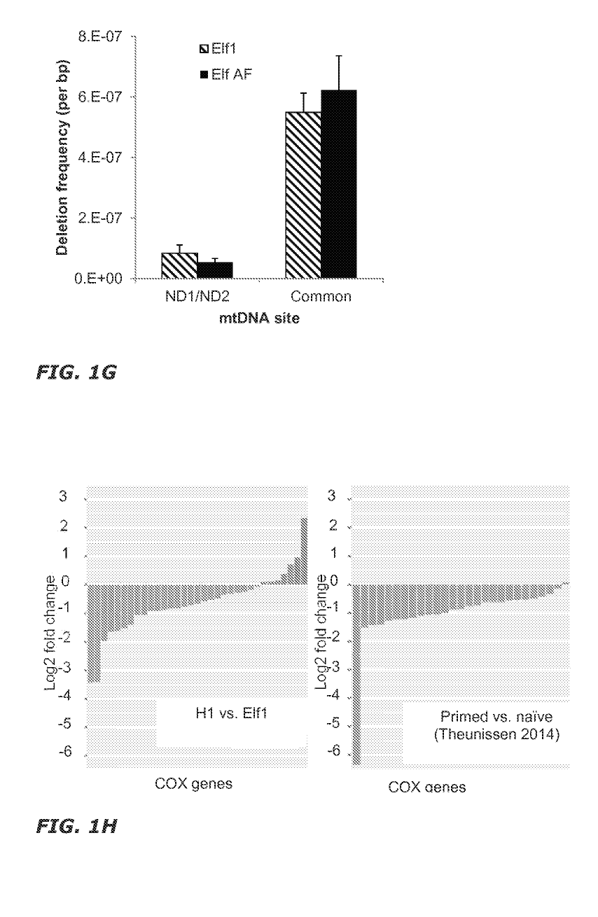

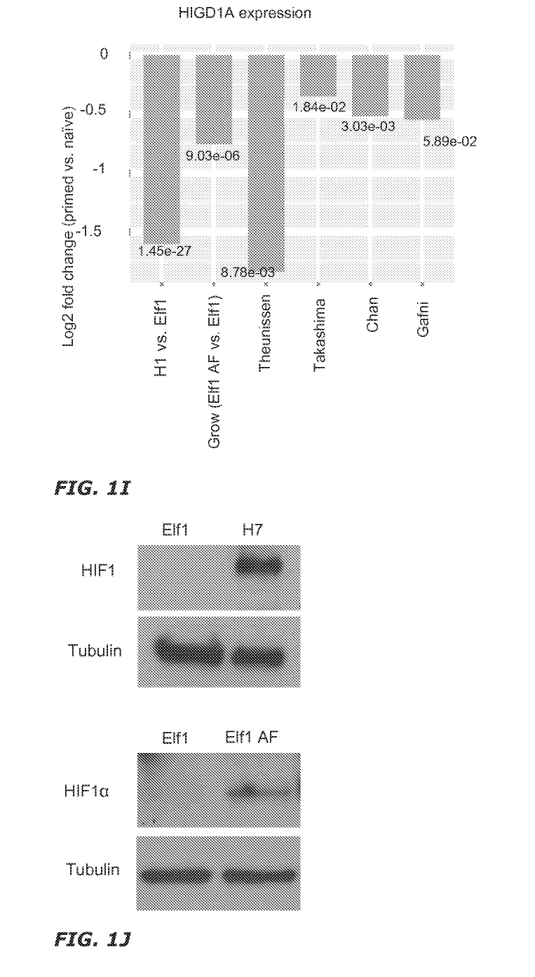

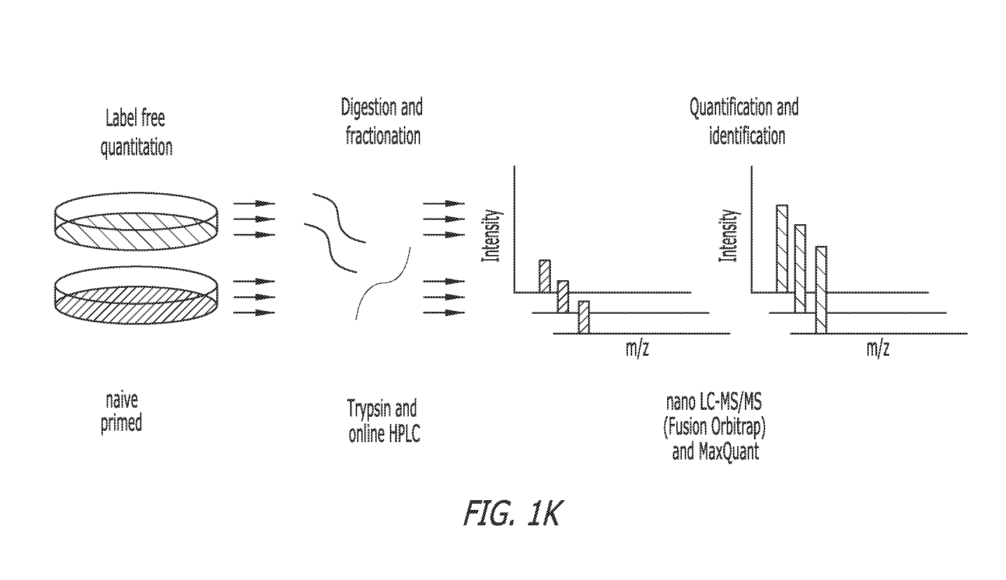

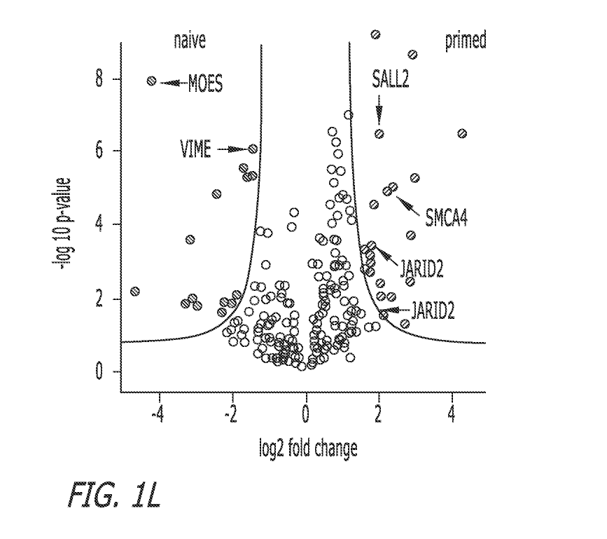

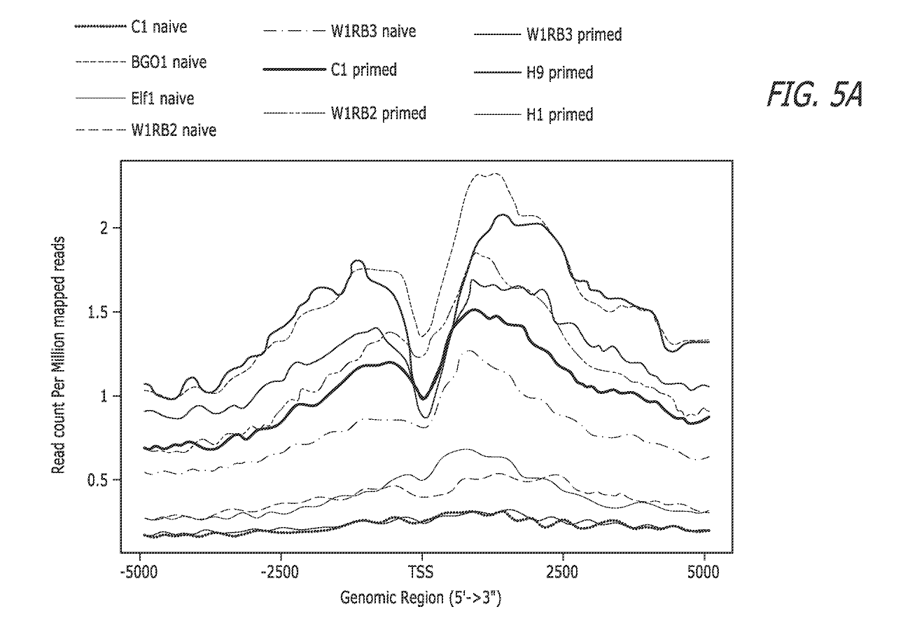

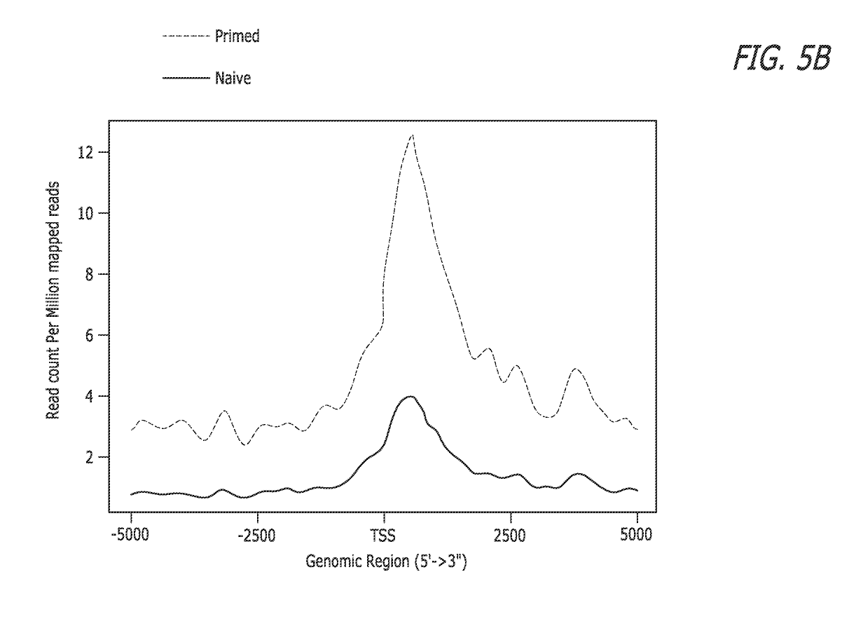

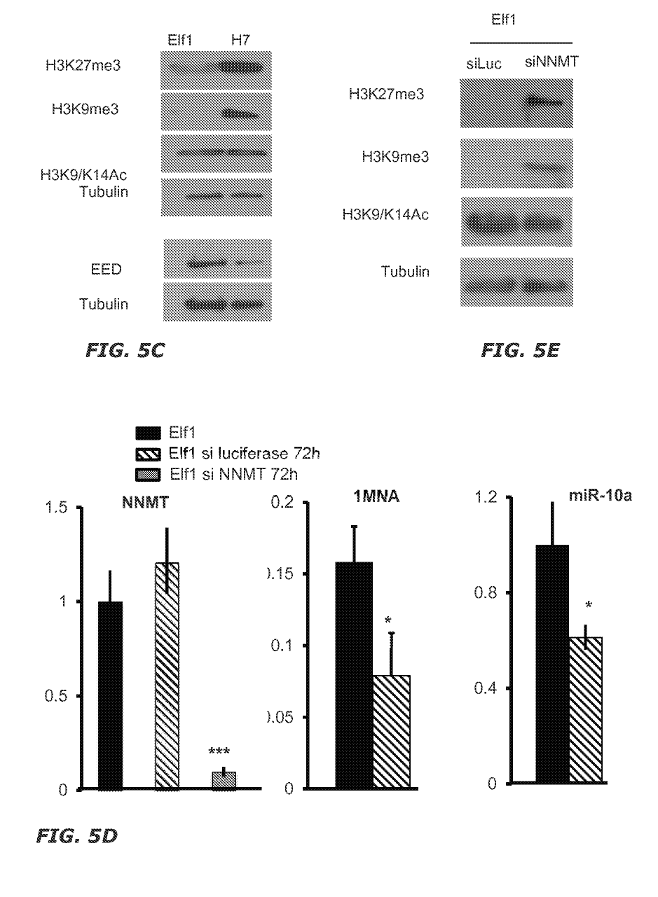

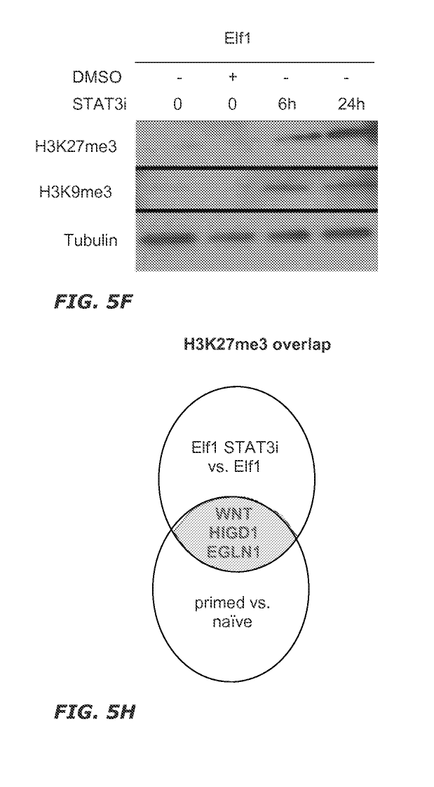

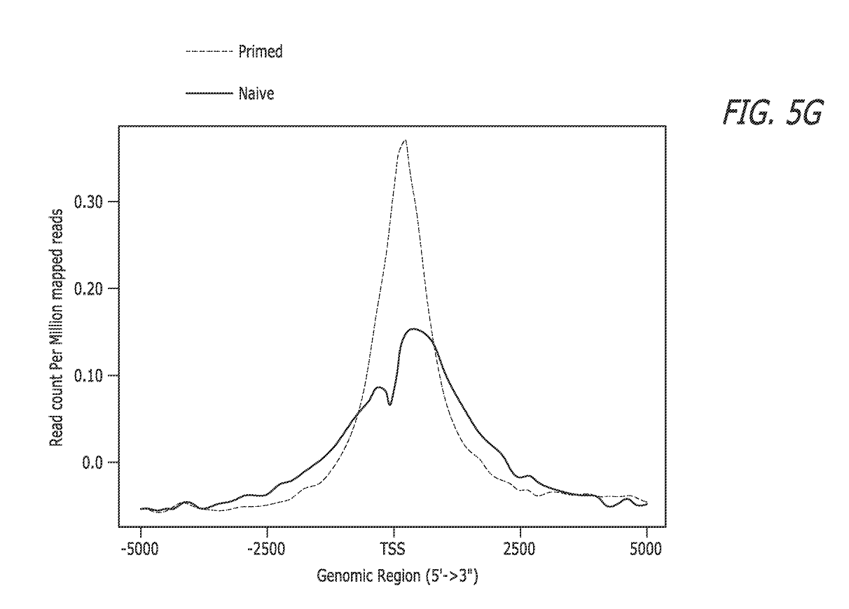

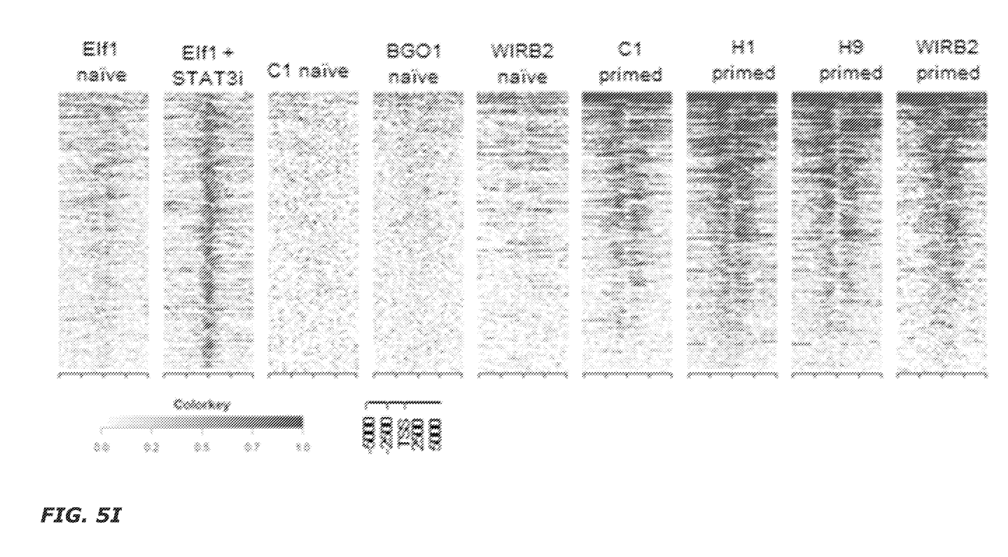

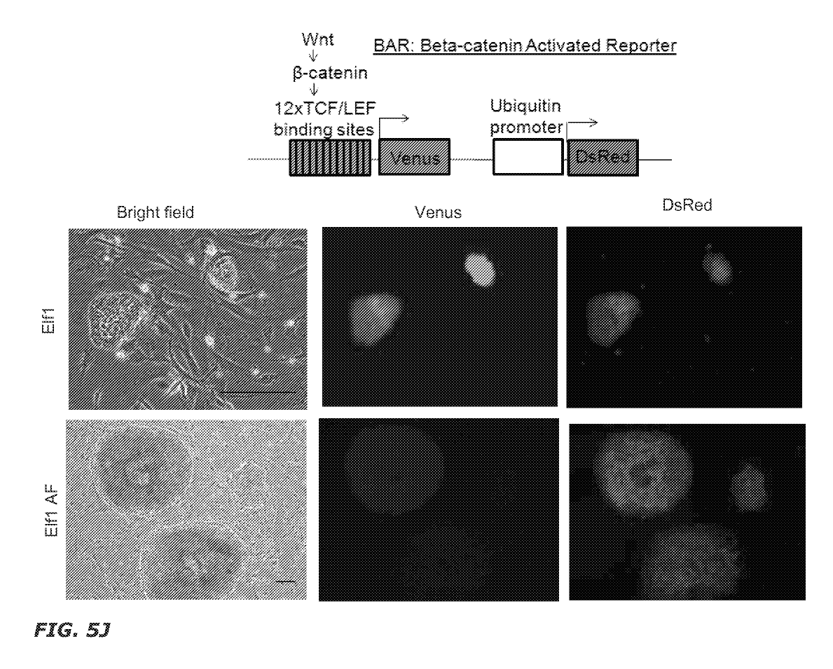

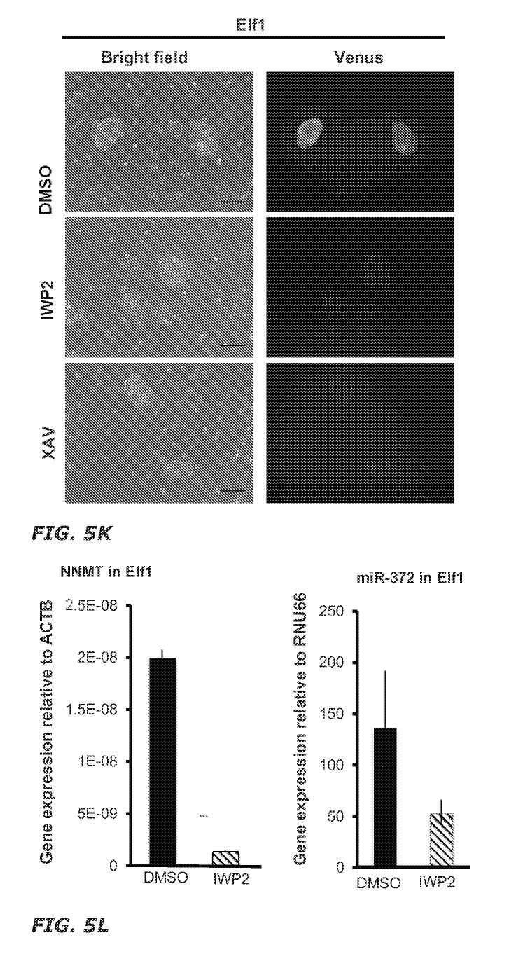

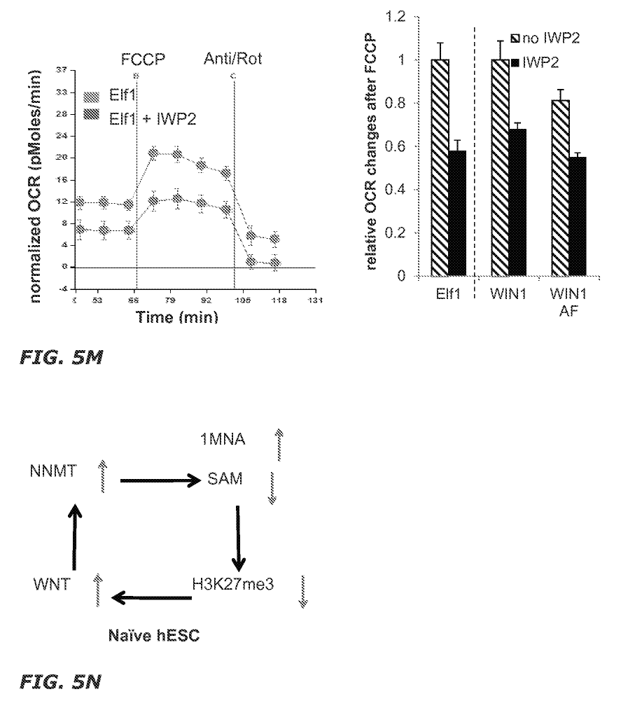

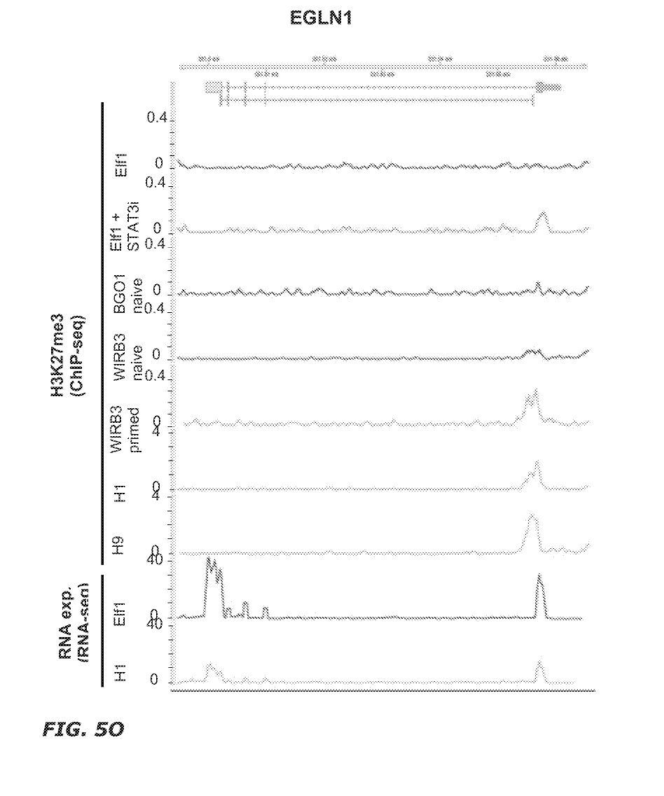

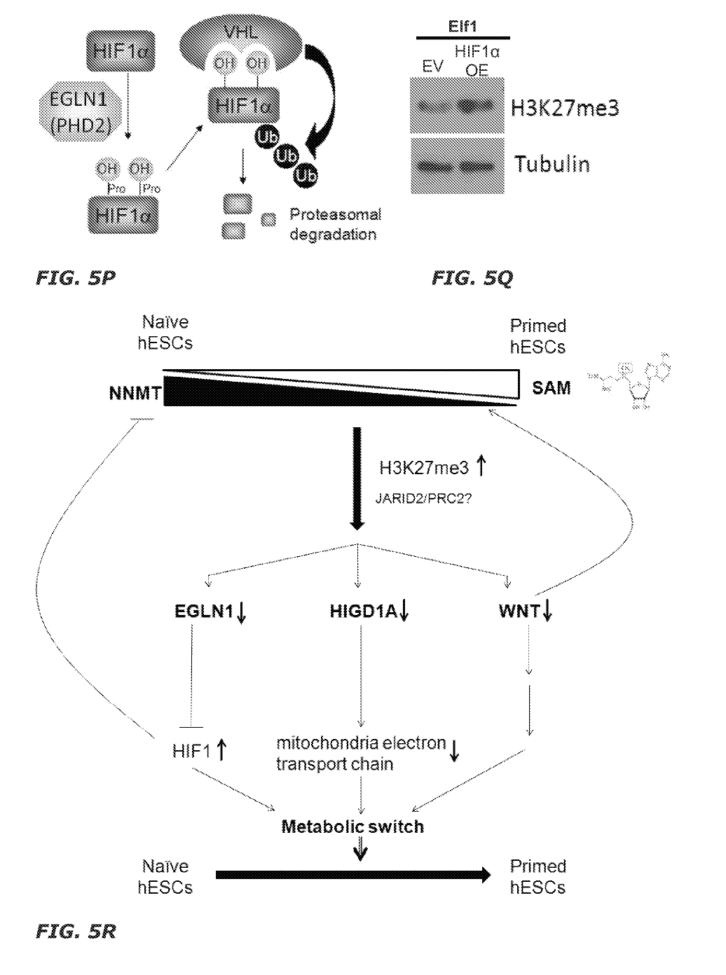

While H3K27me3 marks are reduced in naïve compared to primed hESCs, the enzymes required for this methylation, EZH2/EED, (enhancer of zeste homolog 2/end-to-end polycomb protein) are not downregulated. High NNMT activity in naïve hESCs sequesters the methylation substrate, SAM, thereby repressing the H3K27me3 mark. Furthermore the PRC2 recruiting protein JARID2 is upregulated in primed hESCs compared to naïve, which may give further specificity to PRC2 action in naïve to primed hESC transition (FIG. 1L-1M, FIG. 5R).

SAM levels and NNMT function directly impact histone marks in naïve hESCs, also demonstrated in knockdown experiments, revealing that changes in the metabolic profile of hESCs shape the epigenetic landscape during hESC development. Upregulation of the metabolite SAM accelerates the naïve to primed hESC transition. SAM availability is normally limited by high NNMT activity in naïve hESCs. NNMT knockdown results in reduction of naïve hESC enriched microRNAs as well as an increase in H3K27me3 patterns, both indications of transition towards the primed hESC state (FIG. 5D-5E). The key early responsive targets to STAT3 inhibition, and thereby NNMT reduction are EGLN (HIF inhibitor), Wnt ligands, and HIGD1A (electron transport supercomplex regulator) (FIG. 5H). Wnt and HIF pathways are regulated by metabolite levels in the naïve to primed hESC transition. Inhibition of the Wnt-pathway in the naïve stage reduces NNMT and naïve enriched microRNA-372 levels (FIG. 5L) and furthermore accelerates the metabolic switch towards the primed stage disrupting the feedback loop required to maintain naïve hESC stage (FIG. 5M-5N). Similarly, stabilization of HIF1 reduces naïve and increases primed hESC markers (FIG. 5Q). The availability of SAM triggers the cascade by activating PRC2 and thereby increasing repressive H3K27me3 epigenetic marks in the promoters of key regulators of naïve to primed transition, HIF repressor, Wnt ligands and electron transport chain supercomplex stabilizer (FIG. 5R). The combinatorial action of these key regulators is required for the naïve to primed hESC metabolic transition.

Thus, disclosed herein are methods of detecting the developmental state of a stem cell. The developmental state of the stem cell is determined by measuring the levels of one or more metabolites in a culture of stem cells. Naïve state stem cells have higher levels of the one or more naïve state metabolites than the primed state stem cells, and primed state stem cells have higher levels of one or more primed state metabolites than the naïve state stem cells.

Primed and naïve stem cells have different characteristics that indicate that the cells can be used in different applications. Naïve state stem cells may have a greater potential for differentiation and maturation into different cell types as they may be more plastic than primed state cells. Prime state stem cells, on the other hand, are better studied and already have a wide range of established differentiation protocols.

As used herein, the term “stem cell”, when not specifically referring to an embryonic stem cell, includes hESCs as well as induced pluripotent stem cells (iPSC), germline stem cells, adult stem cells, hematopoietic stem cells and dental pulp stem cells.

As used herein, the term “human embryonic stem cell” or “hESC” refers to human pluripotent stem cells derived from the inner cell mass of a blastocyst, an early-stage preimplantation embryo. Embryonic stem cells are distinguished by their ability to differentiate into any cell type and by their ability to propagate. Embryonic stem cell's properties include having a normal karyotype, maintaining high telomerase activity, and exhibiting remarkable long-term proliferative potential. As used herein, hESCs encompass hESC cell lines and freshly isolated hESCs.

As used herein, the term “primed” or “primed state”, when used in the context of stem cells, refers to stem cells which rely primarily, or solely, on glucose as an energy source and which are not substantially mitochondrially active. Primed stem cells exhibit increased lipid biosynthesis, reduced beta-oxidation, reduced (or substantially eliminated) mitochondrial oxygen consumption, and upregulation of nicotinamide and SAM. Primed state stem cells exhibit a kythenurine/tryptophan ratio of greater than about 0.015.

As used herein, the term “naïve” or “naïve state”, when used in the context of stem cells, refers to stem cells which rely primarily, or solely, on mitochondria as a source of energy. Naïve stem cells exhibit upregulation of NNMT and 1-MNA. Naïve state stem cells exhibit a kythenurine/tryptophan ratio of less than about 0.015.

As used herein, the term “metabolite” refers to a product of cellular metabolism, or a compound which is an agonist or antagonist of a product of cellular metabolism. Metabolites include, but are not limited to, a breakdown product of tryptophan, tryptophan, S-adenosyl methionine (SAM), succinate, fructose (1,6/2,6)-biphosphonate, lactate, methionine, nicotinamide, a long carbon chain lipid, kynurenine, an aryl hydrocarbon receptor (AHR) ligand, an inhibitor or inducer of indoleamine 2,3-diozygenase 1 (IDO1), an inhibitor or inducer of IDO2, an inhibitor or inducer of tryptophan 2,3-dioxygenase 2 (TDO2), an inhibitor or inducer of nicotinamide-N-methyl-transferase (NNMT), an inhibitor of glycogen synthase kinase 3 (GSK3), an inhibitor of mitogen-activated protein kinase (MEK), 1-methylnicotinamide (1-MNA), or S-adenosyl homocysteine (SAH).

Exemplary primed state metabolites include, but are not limited to, a breakdown product of tryptophan, SAM, succinate, fructose (1,6/2,6)-biphosphonate, lactate, methionine, nicotinamide, kynurenine, a long carbon chain lipid, an AHR ligand, an inducer of IDO1, an inducer of IDO2, an inducer of TDO2, or an inhibitor of NNMT.

Exemplary naïve state metabolites include, but are not limited to, a GSK3 inhibitor, a MEK inhibitor, 1-MNA, tryptophan, SAH, an inhibitor of IDO1, an inhibitor of IDO2, an inhibitor of TDO2, an inducer of NNMT, a JNK inhibitor, or a p38 inhibitor.

Breakdown products of tryptophan include, but are not limited to, indole and pyruvic acid.

Long carbon chain lipids, as disclosed herein as metabolites, include, but are not limited to, lipids having carbon chains between 25 and 75 carbons. In certain embodiments, the long carbon chain lipids have carbon chains longer than 25 carbons, longer than 30 carbons, longer than 35 carbons, longer than 40 carbons, longer than 45 carbons, or longer than 50 carbons.

Aryl hydrocarbon receptor (AHR) ligands include, but are not limited to, a halogenated aromatic hydrocarbon such as a polychlorinated dibenzodioxin (2,3,7,8-tetrachlorodibenzo-p-dioxin) (TCDD), a dibenzofuran, a biphenyl, a polycyclic aromatic hydrocarbon such as 3-methylcholanthrene, a benzo(a)pyrene, a benzanthracene, or a benzoflavone, a derivative of tryptophan such as an indigo dye or indirubin, a tetrapyrrole such as bilirubin, an arachidonic acid metabolite such as lipoxin A4 or prostaglandin G, a modified low-density lipoprotein, or a dietary carotenoid.

Exemplary dietary carotenoids include, but are not limited to a zanthophyll, a carotene, lycopene, α-carotene, β-carotene, lycopersene, phytofluene, hexahydrolycopene, torulene, α-zeacarotene, alloxanthin, cynthiaxanthin, pectenoxanthin, cryptomonaxanthin, crustaxanthin, gazaniaxanthin, OH-chlorobactene, loroxanthin, lutein, lycoxanthin, rhodopin, rhodopinol (warmingol), saproxanthin, zeaxanthin, oscillaxanthin, phleixanthophyll, rhodovibrin, spheroidene, diadinoxanthin, luteoxanthin, mutatoxanthin, citroxanthin, zeaxanthin furanoxide, neochrome, foliachrome, trollichrome, vaucheriaxanthin, rhodopinal, warmingone, torularhodinaldehyde, torularhodin, torularhodin methyl ester, astacene, astaxanthin, canthaxanthin (aphanicin), chlorellaxanthin, capsanthin, capsorubin, cryptocapsin, 2,2′-diketospirilloxanthin, echinenone, 3′-hydroxyechinenone, flexixanthin, 3-OH-canthaxanthin (adonirubin, phoenicoxanthin), hydroxyspheriodenone, okenone, pectenolone, phoeniconone (dehydroadonirubin), phoenicopterone, rubixanthone, sphonaxanthin, astacein, fucoxanthin, isofucoxanthin, physalien, zeaxanthin, siphonein, p-apo-Z-carotenal, apo-2-lycopenal, apo-6′-lycopenal, azafrinaldehyde, bixin, citranaxanthin, crocetin, crocetinsemialdehyde, crocin, hopkinsiaxanthin, methyl apo-6′-lycopenoate, paracentrone, sintaxanthin, actinioerythrin, 3-carotenone, peridinin, pyrrhoxanthininol, semi-a-carotenone, semi-p-carotenone, triphasiaxanthin, retro-carotenoids and retro-apo-carotenoids, eschscholtzxanthin, eschscholtzxanthone, rhodoxanthin, tangeraxanthin, nonaprenoxanthin, decaprenoxanthin, C.p. 450 2-[4-hydroxy-3-(hydroxymethyl)-2-butenyl]-2′-(3-methyl-2-butenyl)-b,b-carotene, C.p. 473 2′-(4-hydroxy-3-methyl-2-butenyl)-2-(3-methyl-2-butenyl)-3′,4′-didehydro-l′,2′-dihydro-b,y-caroten-1′-ol, and bacterioruberin.

Inhibitors of IDO1 and/or IDO2 include, but are not limited to, agents capable of inhibiting the activity (e.g., the oxidoreductase activity) such as NLG919, INCB024360, indoximod, norharmane, a COX-2 inhibitor, 1-methyl-D-tryptophan, alpha-methyl tryptophan, 1-methyl-L-tryptophan, 1-methyl-D/L-tryptophan, phenyl-thiohydantoin-trp (3-(N-phenyl-thiohydantoin)-indole), methyl-thiohydantoin-trp (3-(N-methyl-thiohydantoin)-indole), propenyl-thiohydantoin-trp (3-(N-allyl-thiohydantoin)-indole), 4-(butylamino)-3-hydroxy-2,2-dimethyl-3,4-dihydro-2H-benzo[g]chromene-5,1-0-dione (and hydroquinone derivative), 6-hydroxy-2,2-dimethyl-2H-benzo[g]chromene-5,10-dione (and hydroquinone derivative), napthalen-2-ylmethyl 2-(1H-indol-2-yl)ethylcarbamodithioate, pyridin-2-ylmethyl 2-(1H-indol-2-yl)ethylcarbamodithioate, 3-(1H-imidazol-4-yl)benzenethiol, 4-(1H-imidazol-4-yl)benzenethiol, 2-(1H-imidazol-4-yl)phenol, and β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, 5-bromoindoxyl diacetate, 5-bromo-4-chloroindoxyl 1,3-diacetate, annulin A, annulin B, annulin C, brassinin derivatives, necrostatin 1/methylthiohydantoine-tryptophan (5-(1H-indol-3-ylmethyl)-3-methyl-2-thioxo-4-imidazolidinone 5-(Indol-3-ylmethyl)-3-methyl-2-thio-hydantoin MTH-DL-tryptophan), a naphtoquinone, p-coumarinic acid ((E)-3-(4-hydroxyphenyl)-2-propenoic acid), rosmarinic acid ((2R)-2-[[(2E)-3-(3,4-dihydroxyphenyl)-1-oxo-2-propenyl]]oxyl-3-(3,4-dihydroxyphenyl)propanoic acid), and epigallocatechin. Additional examples of IDO1 and IDO2 inhibitors are disclosed in WO2008/115804, WO2007/050963, WO2004/093871, and WO2004/094409, which are incorporated by reference herein for all they disclose regarding IDO1 and IDO2 inhibitors. Exemplary nonlimiting COX-2 inhibitors include etoricoxib, celecoxib, rofecoxib, and meloxicam.

Inducers of IDO1 and/or IDO2 include, but are not limited to, interferon gamma, interferon alpha, interferon beta, lipopolysaccharide, dioxin, a Toll-like receptor (TLR), a TLR ligand, a TLR4 agonist, and a TLR9 agonist such as a CpG-oligonucleotide such as those disclosed in US2004005154, U.S. Pat. No. 6,194,388, U.S. Pat. No. 6,207,646, U.S. Pat. No. 6,239,116, U.S. Pat. No. 6,339,068, U.S. Pat. No. 6,406,705, U.S. Pat. No. 6,426,334 U.S. Pat. No. 6,476,000, US2002/0086295, US2003/0212028, and US2004/0248837, all of which are incorporated by reference for all they disclose regarding TLR agonists.

Inhibitors of TDO2 include, but are not limited to, 680C91 ((E)-6-fluoro-3-[2-(3-pyridyl)vinyl]-1H-indole), 709W92 ((E)-6-fluoro-3-[2-(4-pyridyl)vinyl]-1H-indole), sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole), tolmetin (2-[1-methyl-5-(4-methylbenzoyl)-pyrrol-2-yl]acetic acid), diethyl maleate, and L-buthionine-(S,R)-sulfoximine.

Inducers of TDO2 include, but are not limited to, high dietary fat, estrogen, progesterone, and 8-bromoadenosine-cAMP.

Inhibitors of NNMT include, but are not limited to, 1-MNA, depsipeptide, and NMMT inhibitors disclosed in WO 2012/0684863 which is incorporated by reference for all it discloses regarding NNMT inhibitors.

Inducers of NNMT include, but are not limited to, nicotinic acid, interleukin 6, and STAT3.

Inhibitors of GSK3 include, but are not limited to, beryllium, copper, lithium, mercury, tungsten, 6-BIO, dibromocantharelline, hymenialdesine, an indirubins, a meridianin, an aminopyrimidines, CT98014, CT98023, CT99021, TWS119, SB-216763, SB-41528, AR-A014418, AZD-1080, alsterpaullone, cazpaullone, kenpaullone, an aloisine, manzamine A, palinurine, tricantine, TDZD-8, NP00111, NP031115, tideglusib, HMK-32, CHIR99021, and L803-mts.

Inhibitors of MEK include, but are not limited to, trametinib (GSK1120212), selumetinib, binimetinib (MEK162), PD-325901, cobimetinib (XL518), CI-1040, U0126-EtOH, PD98059, BIX 02189, pimasertib, BIX 02188, TAK-733, AZD8330, PD318088, honokiol, SL-327, and refametinib.

Methods of measuring metabolites in cultures of stem cells include, but are not limited to, normal phase liquid chromatography, hydrophilic interaction chromatography (HILIC), time of flight gas chromatography (GC-TOF), and triple quad liquid chromatography (LC-QQQ-MS). There are also protocols to measure specific metabolites, such as the use of Ehrlich's reagent to detect kynurenine as are known to persons of ordinary skill in the art.

In certain embodiments, primed stem cells are differentiated from naïve stem cells based upon a ratio of the metabolites kynurenine and tryptophan in the spent culture media of cells which have not been supplemented with kynurenine or tryptophan. A kynurenine/tryptophan ratio less than or equal to 0.015 is indicative of naïve stem cells and a kynurenine/tryptophan ratio more than 0.015 is indicative of primed stem cells.

In other embodiments, methods are provided for promoting the transition of stem cells from a naïve state to a primed state, or from a primed state to a naïve state. The claimed transition promotion methods comprise culturing the stem cells in a media supplemented with one or more metabolites specific for the desired state. Thus, a method of promoting the transition of stem cells from a naïve state to a primed state comprises culturing naïve stem cells in a cell culture media supplemented with at least one primed state metabolite. Further, a method of promoting the transition of stem cells from a primed state to a naïve state comprises culturing primed stem cells in a cell culture medium supplemented with at least one naïve state metabolite. Confirmation that a transition from primed to naïve state, or from naïve to primed state, is performed by determining the ratio of kynurenine/tryptophan in the culture medium. A kynurenine/tryptophan ratio less than or equal to 0.015 is indicative of naïve stem cells and a kynurenine/tryptophan ratio more than 0.015 is indicative of primed stem cells.

In certain embodiments, the cells are cultured under the transition conditions for at least about 2-7 days to induce a transition from a primed state to a naïve state, or from a naïve state to a primed state. In some embodiments, the cells are cultured for about at least 3 days, about at least 4 days, about at least 5 days, or about at least 6 days to induce a transition from a primed state to a naïve state, or from a naïve state to a primed state.

Suitable cell culture media include any media capable of supporting stem cells. Exemplary media include, but are not limited to, DMEM, DMEM/F-12, and mTeSR™ (Stemcell Technologies). Additionally, cultures of stem cells may have additional additives to promote health of the cells including, but not limited to, basic fibroblast growth factor (bFGF), a histone deacetylase inhibitor (e.g., vorinostat, butyrate), activin A, leukemia inhibitory factor (LIF), a Rho-associated protein kinase (ROCK) inhibitor, (e.g., Y-27632, B-RAF, H-4-023), transforming growth factor beta (TGFβ), insulin-like growth factor (IGF-1), serum, amino acids, etc.

The addition of a primed state stem cell metabolite or a naïve state stem cell metabolite to a culture for promoting a state transition comprises adding a concentration of the metabolite to the culture which is sufficient to promote the desired transition. The concentration of supplemented metabolite is dependent on the metabolite and can be determined by persons of ordinary skill in the art without undue experimentation. In certain embodiments, the concentration of supplemented metabolite is two times the concentration that does not induce a transition. In other embodiments, the concentration of supplemented metabolite is two times the concentration present in a non-supplemented media.

Also disclosed are methods of maintaining stem cells in a naïve or a primed state. In one embodiment, the maintenance method comprises culturing naïve stem cells in a culture medium supplemented with one or more naïve state metabolites to maintain the naïve state. In certain aspects of this embodiment, the concentration of the one or more naïve state metabolites is maintained in the culture medium at the desired concentration by addition of one or more naïve state metabolite or exchange of culture medium containing a lower concentration of the one or more naïve state metabolite.

In another embodiment, the maintenance method comprises culturing primed stem cells in a culture medium supplemented with one or more primed state metabolites to maintain the primed state. In certain aspects of this embodiment, the concentration of the one or more primed state metabolites is maintained in the culture medium at the desired concentration by addition of one or more primed state metabolites or exchange of culture medium containing a lower concentration of the one or more primed state metabolites.

Also disclosed are methods of inhibiting the transition of stem cells from a naïve state to a primed state comprising culturing naïve state stem cells in a culture medium supplemented with one or more naïve state metabolites. In certain aspects of this embodiment, the concentration of the one or more naïve state metabolites is maintained in the culture medium at the desired concentration by addition of one or more naïve state metabolites or exchange of culture medium containing a lower concentration of the one or more naïve state metabolites.

Also provided herein are methods of inhibiting the transition of stem cells from a primed state to a naïve state comprising culturing primed state stem cells in a culture medium supplemented with one or more primed state metabolites. In certain aspects of this embodiment, the concentration of the one or more primed state metabolites is maintained in the culture medium at the desired concentration by addition of one or more primed state metabolite or exchange of culture medium containing a lower concentration of the one or more primed state metabolite.

Furthermore, the methods disclosed herein provide substantially homogeneous populations of primed or naïve state stem cells. As used herein, the term “substantially” refers to more than about 75% of the cells having the desired characteristic. In other embodiments, substantially homogeneous populations comprise populations in which more than about 75%, more than about 80%, more than about 85%, more than about 90%, or more than about 95% of the cells having the desired characteristic, such as a primed or naïve state stem cell as evidenced by kynurenine/tryptophan ratio.

Thus, also disclosed are substantially homogeneous populations of naïve state stem cells produced by the disclosed methods and compostions comprising the substantially homogeneous populations. Also disclosed are substantially homogeneous populations of primed state stem cells produced by the disclosed methods and composition comprising the substantially homogeneous populations.

The disclosed substantially homogeneous populations of naïve state or primed state stem cells are useful in cellular regeneration therapy in which the substantially homogenous populations of stem cells are provided to a subject in need of cellular regeneration. Diseases and disorders amenable to cellular regeneration therapy include diabetes, cardiovascular disease, neurodegenerative diseases, spinal cord injury, brain injury, various aspects of aging, wound healing, and dental disorders.

For cellular regeneration therapy, substantially homogeneous populations of primed or naïve stem cells are provided in a suitable diluent and injected into a target site of a subject, with our without supplementary growth factors. Formulations for the stem cell compositions are known to persons of ordinary skill in the art.

### EXAMPLES

The following materials and methods are used in the experiments described infra.

Culture of Primed and Naïve Embryonic Stem Cells

Primed human embryonic stem cells (ESCs) [H1 (WA-01) and H7 (WA-07)] and naïve hESCs [Elf-1(NIHhESC-12-0156) and WIN1 (NIHhEDC-14-0299)] were cultured on a feeder layer of irradiated primary mouse embryonic fibroblasts (MEF) in hESC media: high glucose (3.151 g/L) DMEM/F-12 media supplemented with 20% knock-out serum replacer (KSR), 1 mM sodium pyruvate, 0.1 mM nonessential amino acids (NEAA), 50 U/ml penicillin, 50 μg/ml streptomycin (all from Invitrogen) and 0.1 mM β-mercaptoethanol (Sigma-Aldrich). hESC media was supplemented with 4 ng/ml basic fibroblast growth factor (bFGF) for primed hESCs and 1 μM GSK3 inhibitor (CHIR99021, Selleckchem), 1 μM of MEK inhibitor (PD0325901, Selleckchem), 10 ng/mL human leukemia inhibitory factor (LIF, Chemicon), 5 ng/mL IGF-1 (Peprotech), 10 ng/mL bFGF for naïve hESCs (Elf1 2iL-I-F). WIN1 naïve cells were cultured in DMEM/F12, Neurobasal (Invitrogen), N-2 supplement (Invitrogen;), B-27 supplement (Invitrogen), 1 mM glutamine, 1% NEAA, 0.1 mM β-mercaptoethanol, penicillin-streptomycin, 50 μg/ml BSA (Sigma), 1 μM GSK3 inhibitor (CHIR99021), 1 μM of MEK inhibitor (PD0325901), 10 μM ROCK (Rho-associated protein kinase) inhibitor (Y-27632, Stemgent), 0.5 μM B-RAF (serine/threonine-protein kinase B-Raf) (SB590885, R&D systems), 1 μM SRC inhibitor (sarcome proto-oncogene) (WH-4-023, A Chemtek), 20 ng/mL human LIF, activin A (Peprotech, 20 ng/ml) and 8 ng/mL bFGF (WIN 1 5iL-A-F6 media). When started, naïve WIN1 cells were cultured without FGF (WIN1 5iL-A). One passage prior to the experiments, the cells were transferred to growth factor reduced MATRIGEL® (Becton Dickinson) in MEF conditioned media (CM). Dispase and Trypsin/EDTA (Invitrogen) were used to passage primed and naïve hESCs, respectively.

To reverse toggle to naïve state, H1 hESCs were first cultured for 3 passages in presence of HDAC (histone deacetylase) inhibitors [50 nM SAHA [vorinostat, suberanilohydroxamic acid] (Cayman) and 0.1 mM butyrate (Sigma-Aldrich)], followed by 1 μM CHIR99021, 1 μM PD0325901 and 10 ng/mL bFGF (basic fibroblast growth factor) (H1 2iF) for 3 passages. Alternatively, H1 cells were pushed toward a more naïve state by culture in 4iLTF media (1 μM GSK3 inhibitor (CHIR99021), 1 μM of MEK inhibitor (PD0325901), 10 μM JNK (Janus N-terminal kinase) inhibitor (SP600125, Selleck), 10 μM p38 inhibitor (SB203580, Selleck), 10 ng/mL human LIF, 5 ng/mL TGFβ (transforming growth factor beta) and 10 ng/mL bFGF or modified protocol where TGFβ was replaced with 5 ng/mL IGF (4iLIF) for 3 passages. H7 cells were also toggled toward naïve state using protocol with 5iL-A-F media (Theunissen 2014) or modified protocol with IGF instead of activin A (H7 5iL-I-F) for 15 passages. In addition, human naïve cells (Elf1 and WIN1) were pushed toward a more primed state by culturing them in bFGF (10 ng/mL) with or without activin A (10 ng/mL) for 3 passages (Elf1 AF, WIN1 AF, WIN1 F cells).

Naïve mouse ESCs (R1) were cultured in DMEM media supplemented with 20% FBS (ES qualified, Invitrogen), 1.5 μM CHIR99021 and 1 μM PD0325901 and mouse LIF at 1,000 units/mL (Chemicon). Primed mouse ESCs (EpiSCs) were cultured in hESC media supplemented with activin A (10 ng/mL) and bFGF (10 ng/mL). mESC R1 were toggled to a primed state with addition of activin A (10 ng/mL) and bFGF (10 ng/mL) (R1 AF) for 3 passages.

All cells were grown at 37° C., 5% CO₂ and 5% O₂.

OCR and ECAR Measurement Using Seahorse Cellular Flux Assays

Naïve and primed ESCs were seeded in their specific growth media onto 96-well Seahorse plates (Seahorse Bioscience) pre-coated with MATRIGEL® at 25×10⁴ or 40×10⁴ cells/well. Culture media were exchanged for base media (unbuffered DMEM (Sigma D5030) supplemented with sodium pyruvate (Gibco, 1 mM) and with 25 mM glucose (for mito stress assay), 25 mM glucose and 50 μM carnitine (for palmitate assay), or 2 mM glutamine (for glucose stress assay)) 1 hr prior to the assay and for the duration of the measurement. Substrates and selective inhibitors were added during the measurements to achieve final concentrations of glucose (2.5 mM), 4-(trifluoromethoxy)phenylhydrazone (FCCP, 300 nM-500 nM), oligomycin (2.5 nM), antimycin (2.5 μM), rotenone (2.5 μM), palmitate (50 μM in BSA), BSA (bovine serum albumin) and ETO (50 μM).

The mitochondrial stress protocol starts with the measurement of baseline oxygen consumption rate (OCR) and extracellular acidification rate (ECAR) followed by measurement of OCR and ECAR changes in response to addition of oligomycin, FCCP and finally antimycin and rotenone.

Then palmitate assay starts with the measurement of baseline OCR followed by measurement of OCR changes in response to injection of palmitate or BSA (in negative controls) and ETO. The OCR and ECAR values were further normalized to the number of cells present in each well, quantified by Hoechst staining (H033342; Sigma-Aldrich) as measured using fluorescence at 355 nm excitation and 460 nm emission (ENVISION® 2104 Multilabel reader, Perkin Elmer). Changes in OCR and ECAR in response to substrates and inhibitors addition were defined as the maximal change after the chemical addition compared to the last OCR value before the addition.

Mitochondrial DNA Mutation Frequency and Copy Number Analysis

The DNA of Elf1 and H7 cells was isolated using DNAZOL® (Invitrogen) following manufacturers protocol. TAQMAN® primers were used to quantify mitochondrial and genomic DNA. The ratio of mitochondrial DNA (mtDNA) to genomic DNA was determined using a standard curve for each primer. Each reaction contained 2 ng of DNA extract, 1× TAQMAN® Universal PCR Master Mix No AMPERASE® UNG (Applied Biosystems), 500 nM of each primer, and 200 nM of the TAQMAN® probe. Using 7300 Real-Time PCR system (Applied Biosystems), the reactions were amplified at 50° C. for 2 min, 95° C. for 10 min, and then 40 cycles of 15 sec at 95° C. followed by 1 min at 60° C. where the intensity of fluorescence was measured.

Naïve hESCs (Elf1) and primed hESCs (H1 and Elf1 AF) cells were grown in triplicate for mutation analysis. Elf1 were analyzed between passage 19 and 23, Elf1AF were analyzed at passage 25, and H1 at passage 65. All lines were grown on MATRIGEL® for the last passage prior to analysis.

DNA was isolated from hESCs with the DNEASY® Blood and Tissue Kit (QIAGEN) according to kit instructions. Rare mutation-bearing molecules were selectively enriched through endonucleolytic destruction of wild-type target sites. A 100 μL digestion reaction mixture was prepared containing 1 μg of genomic DNA, 1 μL (100 U) of Taq1 (New England Biolabs), and Taq1 reaction buffer (Fermentas) and incubated at 65° C. for 10 hr, with an additional 100 U of Taq1 added to each reaction every hour. Larger scale reactions (5 μg DNA in 400 μL) were prepared for digital deletion detection. After each Taq1 addition, samples were mixed and centrifuged to ensure efficient digestion. Prior to ddPCR, complete cleavage of wild-type Taq1 sites was verified by PCR amplification of the target regions followed by restriction digestion and agarose gel electrophoresis. Following digestion, the reactions were recombined, extracted with phenol/chloroform/isoamyl alcohol (25:24:1, v/v), precipitated with ethanol, and resuspended in 1 mM Tris, pH 8. The final concentration of digested DNA was adjusted to yield less than 3500 positive molecules per μL, which is within the range of linearity for the Poisson calculation. Reaction mixtures (25 μL) contained ddPCR Master Mix (Bio-Rad), 250 nM TAQMAN® probe, 900 nM of each appropriate flanking primer, and 0-1000 ng of Taq1-digested DNA. Reaction droplets were made by applying 20 μL of each reaction mixture to a droplet generator DG8 cartridge (Bio-Rad) for use in the QX100 Droplet Generator (Bio-Rad). Following droplet generation, 38 μL of the droplet emulsion was transferred to a Twin.tec semi-skirted 96-well PCR plate (Eppendorf) and heat-sealed with a pierceable foil sheet. Fragments for point mutation detection and mtDNA copy number measurement were amplified as follows: 95° C. for 10 min, followed by 40 cycles of 94° C. for 30 sec, and 60° C. for 1 min. For digital deletion detection, thermal cycling was as follows: 95° C. for 10 min, followed by 50 cycles of 94° C. for 30 sec, and 63.5° C. for 2 min. The thermally cycled droplets were analyzed by flow cytometry in a QX100™ Droplet Digital™ Reader (Bio-Rad) for fluorescence analysis and quantification of mutation frequencies. Positive (mutation-bearing) and negative droplets were distinguished on the basis of fluorescence amplitude using a global threshold. The number of mutant genomes per droplet was calculated automatically by the accompanying software (QUANTASOFT®, Bio-Rad) using Poisson statistics.

Quantification of point mutation or deletion frequency required ddPCR amplification using two primer sets. The first primer set flanks the test region and measures the concentration of mutation-bearing molecules. The second primer set flanks a region in the mitochondrial genome that bears no restriction recognition sites. This control set measures the concentration of all mtDNA genomes. Because de novo point mutations and deletions are rare, reactions using the different primer sets are run using different dilutions of the digested DNA, and the results normalized against undiluted concentrations during downstream calculations. Point mutation frequency per base pair was calculated by taking the ratio of the normalized concentrations of mutation-bearing mtDNA molecules to the total mtDNA molecules screened, divided by the number of bases per target site. Reactions that yielded <10 positive droplets per well were scored conservatively as having no positives above background.

Similarly, mtDNA copy number was determined using two primer sets: the mtDNA control primer set, and a primer set in the RNaseP gene, a region that occurs twice per diploid genome. mtDNA copy number was calculated by taking the ratio of normalized concentrations of total mtDNA molecules to RNaseP molecules, and multiplying this value by 2 to get the number of mtDNA molecules per cell.

Proteomics

Naïve hESCs (Elf1 2iLIF) and primed hESCs (Elf1 AF) were washed in 1×PBS and flash frozen. Cell pellets were lysed in 1 M urea, 50 mM ammonium bicarbonate, pH 7.8, and heated to 50° C. for 20 min. Cell debris was removed by centrifugation (1000×g for 2 min). Following measurement of protein in the samples, normalized quantities of protein were reduced with 2 mM dithiothreitol (DTT), alkylated with 15 mM iodoacetamide, and digested overnight with a 1:50 ratio of trypsin to total protein. The resulting peptides were desalted on Waters SEP-PAK® C18 cartridges.

Peptides were measured by nano-LC-MS/MS on a Thermo Scientific FUSION™ mass spectrometer. Peptides were separated online by reverse phase chromatography using a heated 50° C. 30 cm C18 column (75 mm ID packed with Magic C18 AQ 3 μM/100 A beads) in a 180 min gradient (1% to 45% acetonitrile with 0.1% formic acid) separated at 250 nL/min. The FUSION™ was operated in the data-dependent mode with the following settings: 60000 resolution, 400-1600 m/z full scan, Top Speed 3 seconds, and an 1.8 m/z isolation window. Identification and label free quantification of peptides was done with MaxQuant 1.5 using a 1% false discovery rate (FDR) against the human Swiss-Prot/TrEMB database downloaded from Uniprot on Oct. 11, 2013. The databases contained forward and reverse mouse sequences as well as common contaminants. Two biological and three technical replicates were analyzed per condition. Peptides were searched using a 5 ppm mass error and a match between run window of 2 min. Proteins that were significantly regulated between conditions were identified using a permutation-based t-test (S1, FDR 5%) in Perseus 1.4.1.3.

Non-Targeted GC-TOF and LC-QTOF Analysis for Metabolites in Mouse and Human ESCs

For the first set of experiment cells (5×10 cm plates for each H1 replicate—equivalent of 5-10 million cells, and 2×10 cm plate for each R1 replicate—equivalent of 10-20 million cells) were scraped in PBS, pelleted in 6 replicates per condition, quick frozen and stored at −80° C., then thawed on ice and mixed with 2 mL of ice-cold degassed acetonitrile followed by vortexing for 20 sec, sonication for 5 min, and vortexing for 20 sec. One milliliter of material was taken and centrifuged for 5 min at 14,000 rcf and the remaining material was saved. Supernatant was divided into a 500 μL (GC-TOF) and a 250 μL (LC-QTOF) aliquot, which were lyophilized. Lyophilized material was resuspended in 500 μL of 1:1 acetonitrile:H₂O and centrifuged for 2 min at 14,000 rcf to remove membrane lipids and triglycerides. Supernatant was collected and lyophilized.

For the second set of experiments 2×10 cm plates of cells were used for each replicate. The cells were scraped, washed in PBS, pelleted, quick frozen and stored at −80° C., then thawed on ice and extracted by adding 225 μL of chilled methanol with internal standard mixture, 750 μL of chilled MTBE (methyl tertiary butyl ether, Sigma Aldrich) containing the internal standard 22:1 cholesteryl ester and 188 pL of LCMS grade water (Sigma Aldrich). After vortexing, samples were centrifuged at 14000 rcf for 2 min to separate phases. The upper layer was used for lipid analysis and bottom layer used for GC analysis, both layers were evaporated to dryness.

GC-TOF MS Extraction and Analysis

GC-TOF analysis and data processing was performed using a Leco Pegasus IV time of flight mass spectrometer (Leco Corporation) coupled to an Agilent 6890 gas chromatograph (Agilent Technologies) equipped with a 30 m long 0.25 mm id Rtx5Sil-MS column and a Gerstel MPS2 automatic liner exchange system (Gerstel GMBH & Co. KG).

LC-QTOF MS Extraction and Analysis for Non-Targeted Analysis

For the first set of experiments lyophilized material was redissolved in 100 μL initial LC gradient solvent and analyzed within 24 hr. HILIC and reversed phase LC-QTOF analysis and data processing was performed using an Agilent 1200 series HPLC equipped with either Agilent ZORBAX® Eclipse Plus C18 2.1×150 mm column for reversed phase or a Waters 1.7 μm ACQUITY® BEH HILIC 2.1×150 mm column. LC eluents were analyzed with an Agilent 6530 accurate mass Q-TOF mass spectrometer. The LipidBlast database was used for identifications.

For the second set of experiments the lipid extracted phase was re-dissolved in 90:10 methanol:toluene (Fisher Scientific) with 50 ng/mL CUDA (12-[[(cyclohexylamino)carbonyl]amino]-dodecanoic acid, Cayman Chemical) and analyzed using an Agilent 1290 Infinity Ultrahigh Pressure Liquid Chromatography stack (Agilent Technologies) equipped with an auto-sampler (40c) using 3 μL and 5 μL injections for positive and negative respectively into an ACQUITY® UPLC CSH C18 column (Waters Corporation) maintained at 65° C. with 1.7 μm particles and 2.1 mm i.d.×100 mm length. Mobile phases were prepared with 10 mM ammonium formate and 0.1% formic acid for positive mode and 10 mM ammonium acetate for negative mode. Both positive and negative modes used mobile phase composition of 60:40 acetonitrile:water for mobile phase A and 90:10 isopropanol:acetonitrile for mobile phase B. Gradient elution was performed from 0 min 15% B, 0-2 min 30% B, 2-2.5 min 48% B, 2.5-11 min 82% B, 11-11.5 min 99% B, 11.5-12 min 99% B, 12-12.1 min 15% B, and 12.1-15 min 15% B with a column flow of 0.6 mL/min. Metabolites were detected and quantified by an Agilent 6550 accurate mass quadrupole time-of-flight (QTOF) mass spectrometer with a jet stream ESI source (Agilent). Mass calibration was maintained by constant reference ion infusion, with MS data collected at 2 spectra/s. Method blanks and human pooled plasma samples were used as QC controls. MZmine 2.10 was used to process the raw data and metabolites were reported when present in 50% of each samples in each group. Annotations were made based on in house accurate mass and retention time library created using LipidBlast.

Multivariate Analysis of Primed vs Naïve Metabolomes

Metabolite measurements of known and unknown GC metabolites and known lipid metabolites (from ESI(+) and ESI(−) modes) were submitted using R to DeviumWeb (v 0.3.2). Metabolites were normalized using unit norm normalization. O-PLS-DA, a multivariate classification model, was used to identify differences between primed and naïve human and mouse cells. Robust model performance statistics were generated by 100 rounds of Monte Carlo cross validation using training and testing compared to the permuted model (random chance) and all plots were mean centered, and used unit variance for scaling. Based on the test/training and permutation testing the selected O-PLS-DA plots showed significantly higher classification performance (P<0.05) compared to the permuted model.

LC-QTOF for Elf1 and HI hESCs

For lipid extraction Elf1 and H1 cells were grown on MATRIGEL®-coated 35 mm plates for one passage. Cells were washed with PBS, followed by a 2 sec wash with 37° C. deionized water and the addition of 0.5 ml of a −75° C. solution and internal standards. The plates were incubated on dry ice for 15 min before scraping the plates and transferring everything into microcentrifuge tubes. One milliliter of chloroform was added to the tubes, followed by another 15 min incubation on dry ice. The mixture was spun for 5 min at 4° C. at 18000 rcf, after which the lower phase was collected and stored at −80° C.

LC-QTOF-MS experiments were performed using an Agilent 1200 SL LC system coupled online with an Agilent 6520 Q-TOF mass spectrometer (Agilent Technologies). Each prepared sample (4 μL for positive ESI ionization, 8 μL for negative ESI ionization) was injected onto an Agilent ZORBAX® 300 SB-C8 column (2.1×50 mm, 1.8-micron), which was heated to 50° C. The flow rate was 0.4 mL/min. Mobile phase A was 5 mM ammonium acetate and 0.1% formic acid in water, and mobile phase B was 5% water in ACN containing 5 mM ammonium acetate and 0.1% formic acid. The mobile phase composition was kept isocratic at 35% B for 1 min, and was increased to 95% B in 19 min; after another 10 min at 95% B, the mobile phase composition was returned to 35% B. The ESI voltage was 3.8 kV.

Targeted LC-QQQ-MS Analysis for Water Soluble Metabolites

Elf1 and Hi cells were grown on MATRIGEL®-coated 35 mm plates (3 plates per replicate) for one passage. Cells were washed with PBS, followed by a 2 sec wash with ice cold deionized water and the addition of a −75° C. 0.75 mL 9:1 methanol:chloroform solution. The plates were incubated on dry ice for 15 min before scraping the plates and transferring the cellular debris into microcentrifuge tubes, which were spun at 18000 rcf for 5 min at 4° C. All soluble extract was transferred into a new tube and vacuum dried. Samples were stored in −80° C. before preparing for MS analysis.

Chromatography conditions: dried samples were reconstituted in 200 μL 5 mM ammonium acetate in 40% water/60% acetonitrile+0.2% acetic acid, and filtered through 0.45 μm PVDF filters (Phenomenex) prior to LC-MS analysis. LC-MS/MS was performed using an Agilent 1260 LC AB-Sciex 5500 QQQ MS 62. Both chromatographic separations were performed in HILIC mode on two SEQUANT® ZIC-cHILIC columns (150×2.1 mm, 3.0 μm particle size, Merck KGaA). While one column was performing the separation, the other column was reconditioned for the next injection. The flow rate was 0.300 mL/min, auto-sampler temperature was kept at 4° C., the column compartment was set at 40° C., and total separation time for both ionization modes was 20 min The mobile phase was composed of Solvents A (5 mM ammonium acetate in 90% H₂O/10% acetonitrile+0.2% acetic acid) and B (5 mM ammonium acetate in 90% acetonitrile/10% H₂O+0.2% acetic acid). The gradient conditions for both separations were identical and were as follows: 0-2 min, 25% A—2-5 min, 25% to 70% A, linear gradient—5-7 min, 70% A—9-11 min 70% to 25% A, linear gradient—11-20 min, The chromatographic separation, MS ionization and data acquisition was performed using an AB SCIEX® QTrap 5500 mass spectrometer equipped with electrospray ionization (ESI) source. The instrument was controlled by Analyst 1.5 software (AB Sciex). Targeted data acquisition was performed in multiple-reaction-monitoring (MRM) mode. Ninety-eight and 59 MRM transitions in negative and positive mode, respectively (157 transitions total) were monitored.

The extracted MRM peaks were integrated using MultiQuant 2.1 software (AB Sciex).

Targeted HILIC-QTOF Mass Spectrometry Metabolite Quantifications of Methionine Metabolites

Cells were grown on MATRIGEL®-coated 10 cm plates for one passage (4 replicates each, 2 plates per replicate). Cells were scraped and washed with PBS at room temperature, pelleted and flash frozen in liquid nitrogen. Samples were extracted by adding 1 mL cold 3:1 methanol:water to the cell pellet. Samples were vortexed, placed at −20° C. for 30 min, and then centrifuged for 10 min at 14000 rcf. The supernatant was transferred then centrifuged again, then supernatant was evaporated to dryness. Samples were resuspended in 80:20 acetonitrile:water containing Val-Try-Val (Sigma). Standard curve dilutions for quantifications were prepared using mixture of 1methylnicotinamide HCl (1-MNA), S-methyl-5′-thioadenosine (MTA), S-adenosyl methionine (SAM), S-adenosyl homocysteine (SAH), methionine, kynurenine, and tryptophan (Sigma). Hydrophilic interaction chromatography (HILIC) analysis of standard curve and samples was performed using an Agilent 1290 Infinity Ultrahigh Pressure Liquid Chromatography stack (Agilent Technologies) equipped with an auto-sampler (4° C.) using 5 μL injections into an ACQUITY® UPLC BEH Amide column (Waters Corporation) maintained at 45° C. with 1.7 μm particles and 2.1 mm i.d. (inner diameter)×150 mm length. Mobile phases were prepared with 10 mM ammonium formate and 0.125% formic acid in either 100% LCMS grade water for mobile phase A or 95:5 acetonitrile:water for mobile phase B. Gradient elution was performed from 100% B at 0-2 min to 70% B at 7.7 min, 40% B at 9.5 min, 30% B at 10.25 min, 100% B at 12.75 min, isocratic until 16.75 min with a column flow of 0.4 mL/min. Metabolites were detected and quantified by an Agilent 6530 accurate mass quadrupole time-of-flight (QTOF) mass spectrometer with a jet stream ESI source in positive ion mode (Agilent). Mass calibration was maintained by constant reference ion infusion, with MS data collected at 4 spectra/s. Data files were analyzed using Agilent Mass Hunter TOE Quantitative Analysis software. Metabolites were quantified at the precursor ion level set at a mass window of 0.02 Da. Peak filtering was performed manually to eliminate peaks with a signal to noise ratio of less than 3. Retention times and major adducts for each compound are as follows: 1-MNA (m/z 137.0715) 6.345 min, MTA (m/z 297.0896) 2.583 min M+H, tryptophan (m/z 204.0899) 6.904 min M+H, kynurenine (m/z 208.0848) 6.971 min. M+H & M+Na, methionine (m/z 149.0511) 7.493 min M+H & M+2Na+H, SAH (m/z 384.1216) 8.810 min M+H, SAM (m/z 399.1451) 9.768 min.

Metabolite levels were sum-normalized for each sample using the methionine metabolite values (nicotinamide, MTA, 1-MNA, SAM and SAH). P-values were calculated using a 1-tailed t-test.

Transcriptomic Data Analysis

To replicate the principle component analysis results in Takashima 2014, RNA-seq data processing was performed according to Takashima 2014. Raw RNA-seq reads from this study generated herein and from three other studies (Chan 2013, Takashima 2014, and Yan 2013) were aligned to hgl9/GRCh37 with STAR aligner. Transcript quantification was performed with htseq-count from HTSeq package using GENCODE v1565. Differential expression analysis was performed with DESeq after filtering out genes whose total read count across samples are below the 40th quantile of all genes.

Size factors used to normalize by library size were computed using the DESeq packagers Reads were further normalized by gene length.

Affymetrix Human Gene Array 1.0 ST arrays (Gafni 2013) were processed with oligo package and normalized using Robust Multi-array Average. Multiple probes mapping into the same gene were summarized into a single expression value by taking the max. Affymetrix PrimeView arrays from Theunissen 2014 were processed with Affy package and normalized with RMA. Microarray differential expression analysis was performed using the limma package. To combine RNA-seq and microarray data from different studies across different platforms, following steps outlined in Takashima 2014, expression levels were converted to log 2 fold change relative to the mean of human embryo-derived PSC samples within each study. One-to-one orthologous genes between mouse-human were mapped in the same way as Takashima 2014. PCA plot of all samples from all studies were generated using the princomp function from R stats package.

Global Metabolomic Data Analysis

AIII global metabolomic data was mean-centered within each sample prcomp function in R (R Core Team, 2013) is used for Principle Component Analysis of metabolomics data. Differentially abundant metabolites were defined as metabolites with 2 fold change in abundance and Benjamini-Hochberg adjusted false discovery rate <0.2.

For the lipidomics data, features missing in more than half of all samples (4 or more out of 6) were removed from further analysis. Missing values were replaced with minimum detected values within each sample before mean-centering.

ChIP-Seq Data Analysis

ChIP-seq data of H3K27me3 H3K4me3, H3K9me3 and H3K27ac modifications from Chan 2013, Gafni 2013, Theunissen 2014, and Bernstein 2010 was downloaded from Array Express (accession number E-MTAB-2041) and GEO (accession numbers GSE52617 and GSE59435). Reads were aligned to hg19 using Bowtie version 1.0.0. allowing 1 mismatch (—N 1). ngsplot was used to generate plots of reads around 5 KB of transcription start sites of a priori defined developmental genes. Reads of replicate samples for the same cell type were merged for ngsplot. Reads with mapping quality above 20 were used by ngsplot. Differentially marked genomic regions were identified with diffReps version 1.55.4 and annotated to the closest genes. Genes associated with at least one significant genomic region (FDR less than 0.05 and fold change >1) were classified as differentially marked. When a gene was annotated with multiple significant genomic regions, the most significant one is assigned to that gene.

Lipid Droplet Visualization Using Oil Red and BODIPY Staining

Naïve and primed ESCs were fixed with 4% PFA at room temperature for 10 min, washed twice with PBS and stained with Oil Red 0 dye (Sigma) for 10 min at room temperature. Alternatively, lipid droplets were stained using BODIPY 493/503 (Molecular Probes) for 15 min on a rocking platform at room temperature. Pictures were taken using a fluorescent microscope (Leica).

Protein Extraction and Western Blot Analysis

Cellular extracts were prepared using a lysis buffer containing 20 mM Tris HCl (pH 7.5), 150 mM NaCl, 15% glycerol, 1% Triton, 25 mM (3-glycerolphosphate, 50 mM NaF, 10 mM sodium pyrophosphate, orthovanadate, PMSF), protease inhibitor cocktail (Roche) and 2% SDS. 25 U of BENZONASE® nuclease (EMD Chemicals) and 20 mM of DTT were added to the lysis buffer right before use. Protein concentrations were then determined by the method of Bradford. Fifteen micrograms of protein extracts were loaded, separated by 4-20% SDS-PAGE, and transferred to polyvinylidene difluoride membranes (Hybond-N+, Amersham Pharmacia Biotech). Membranes were blocked with 5% nonfat dry milk for 60 min at room temperature, and incubated overnight at 4° C. with primary antibody. Finally, after blots had been incubated for one hr with horseradish peroxidase-conjugated secondary antibodies, they were visualized by enhanced chemiluminescence (Millipore Corp.). Antibodies used in this study were specific for: H3K27me3 (1/1000, Active Motif), H3K9me3 (1/1000, Abcam), H3K4me3 (1/1000, Millipore), H3K9/14Ac (1/1000, Cell Signaling), EED (1/1000, gift from Dr. Bomsztyk), HIF1a (1/2000, BD Biosciences), LDHA (1/1000, Cell Signaling), JARID2 (1/1000, Cell Signaling), pSTAT3 (1/1000, Cell Signalling), y-tubulin (1/10000, Sigma) and β-actin (1/5000, Santa Cruz Biotechnology).

RNA Extraction and qPCR Analysis

Total RNA was extracted using TRIZOL® and subsequently analyzed by SYBR® green qPCR with the 7300 real time PCR system (Applied Biosystems) as well as TAQMAN® qPCR (Applied Biosystems).

qPCR of miRNAs was conducted using AQMAN®miRNA assays (Applied Biosystems). Raw Ct (threshold cycle) values for miRNAs were normalized to RNU66 (endogenous snoRNA, internal control). Linear expression values for all qPCR experiments were calculated using the 2 (−ΔCt) method. P-values were calculated using a student's t-test (*p<0.05, **p<0.01, ***p<0.001).

ChIP-Seq Analysis

Naïve hESCs (Elf1 2iLIF) grown on MATRIGEL® were treated with 100 μM of STAT3 inhibitor (Selleckchem) for 6 hr or 24 hr and analyzed for methylation marks by Western blot and ChIP Seq. For ChIP-seq analysis, cells were harvested with ACCUTASE® and crosslinked in suspension with 1% formaldehyde solution for 10 min at room temperature. Reaction was quenched with glycine and crosslinked cells were rinsed with ice-cold PBS. Nuclei were isolated and chromatin sonicated using a Covaris E210 to approximately 200-500 bp size range. ChIP-seq was conducted as previously described with minor modifications. Briefly, magnetic DYNABEADS® were incubated overnight rotating at 4° C. with antibody against H3K27me3 (Active Motif, cat #39155). Sonicated chromatin from approximately 200 thousand cells was added to the bead-bound-antibodies and allowed to incubate at 4° C. rotating overnight. Beads were washed to remove unbound chromatin. Bound chromatin was eluted from beads and reverse crosslinked overnight. Purified DNA was prepared for next-generation sequencing via end repair, A-tailing, ligation of custom Y-adapters and PCR amplification to generate final DNA library following gel size selection.

Generation of BAR-Elf Reporter Cell Line

Elf1 cells grown in naïve media (2iL or 2iLIF) or primed media (AF) were infected with BAR reporter lentivirus and seeded onto MATRIGEL®-coated plates in MEF-CM with 10 μM Y-27632, and 1 μM Thiazovivin (ROCK inhibitors, Tocris). Transduced Elf cells were cultured for a week on MATRIGEL®, then passaged onto MEF plates for further selection and expansion. Elf1 naïve reporter cells were harvested as single cells via TRYPLE™ Express and FACS sorted for the population with both Venus and DsRed (Discosoma sp. Red fluorescent protein) positive signals. DsRed positive colonies of Elf1 primed reporter cells were manually dissected, transferred onto MEF plates, and the same positive selection was repeated one more round or two depending on the selection efficiency. Negative colonies were manually removed as a negative selection. Once the DsRed positive lines were isolated and stable lines established, the primed Elf1 reporter cells were passaged with dispase.

Manipulation of Wnt Pathway

Wnt secretion and signaling were inhibited in naïve hESCs (Elf1, WIN1) by treatment with IWP2 (2 μM, Torcis) or XAV939 (5 μM, Sigma). Wnt pathway was activated in primed Elf1 AF reporter cells using a GSK3 inhibitor, CHIR99021 (72 hr, 10 μM, AxonMedChem). Both IWP2 and CHIR99021 were reconstituted in DMSO.

Production of Conditioned Medium (LCM and Wnt3A-CM)

L and L-Wnt3A cells (ATCC) were cultured in 15 cm plates in 10% FBS/DMEM media until 90% confluent. Medium was collected every 48 hr for three batches. Biological activity of secreted Wnt3A in the individual batches of the conditional medium was confirmed in 293T-BAR reporter cells, then batches were pooled and filtered. Primed (Elf1 AF) reporter cells were grown on MATRIGEL® with 50% LCM or 50% Wnt3A-CM for 3 days prior taking bright field and fluorescent pictures.

RNA Interference Experiments

Naïve Elf1 2iLIF cells were transfected in MATRIGEL® coated plates in MEF-CM supplemented with ROCK inhibitors (Torcis) using LIPOFECTAMINE™ RNAiMAX™ (Life Technologies). siRNA targeting NNMT (Hs-NNMT-8) was purchased from Qiagen as FLEXITUBE® siRNA premix, and siRNA targeting luciferase was used as control. siRNAs against NNMT and luciferase were used at 50 nM final concentration. Proteins and RNAs were extracted 72 hr after transfection. siRNA targeting beta-catenin (Invitrogen, CTNNBI, SILENCER® Select ID s437) and SILENCER® Select Negative Control 1 (Invitrogen) were transfected in naïve Elf1 2iLIF cells at 10 nM final concentration following a reverse transfection protocol. Bright field and fluorescence images were taken after 3 days. The efficacy of NNMT and beta-catenin siRNAs was confirmed by qPCR analysis.

Overexpression of NNMT

Naïve hESCs Elf1 were transfected with NNMT overexpression construct or inactive NNMT mutant overexpression construct (Y20A). Cells were plated the following day into Seahorse plate coated with MATRIGEL® with primed hESC media (conditioned media+AF) and a mitostress protocol in Seahorse flux analyzer was performed 2 days later.

Detection of Kynurenine in Media

The biological activity of indoleamine 2,3-diozygenase (IDO) was evaluated by measuring the level of tryptophan degradation product, L-kynurenine, present in the medium of primed and naïve hESCs. Proteins in the medium were precipitated with 30% trichloroacetic acid. After centrifugation at 8000×g for 5 min, the supernatant was incubated with an equal volume of Ehrlich's reagent (1% 4-dimethylaminobenzaldehyde in glacial acetic acid) at room temperature for 10 min. Optic density was measured at 492 nm, using a Molecular Devices SPECTRAMAX® Plus 384 microplate reader. The concentration of kynurenine in the medium was calculated according to a standard curve of defined kynurenine concentration (0-100 NM). P-values were calculated using a t-test (*p<0.05)

Treatment with Metabolites SAM, 1MNA and Kynurenine

WIN1 and Elf1 cells were seeded in Seahorse plates in naïve (WIN1, Elf1) or primed (WIN1 AF) conditions with or without Kynurenine (100 μM, Sigma) and mitostress protocol was performed 2 days later.

WIN1 cells were seeded in Seahorse plates 2 days prior change of media with media without L-methionine (Sigma 0422 supplemented with 0.584 gm/L L-glutamine) and addition of SAM (500 μM). Five hours later Seahorse mitostress protocol was performed.

Primed hESCs (Elf1 AF) were treated with 1-MNA (0.5 mM) in media with low L-methionine (Sigma 0422 supplemented with 0.584 gm/L L-glutamine and 10 μM L-methionine) for 3 days before protein extraction.

HIF1a Overexpression in Naïve hESCs

Naïve hESCs (Elf1) were infected with a non-degradable form of HIF1a overexpressing construct (Addgene plasmid 19005, Yan et al., Mol. Cell. Biol. 27:2092-2102, 2007) or a pBABE empty vector construct in presence of 4 ng/ml polybrene. RNA and proteins were harvested 24 days later.

### Example 1

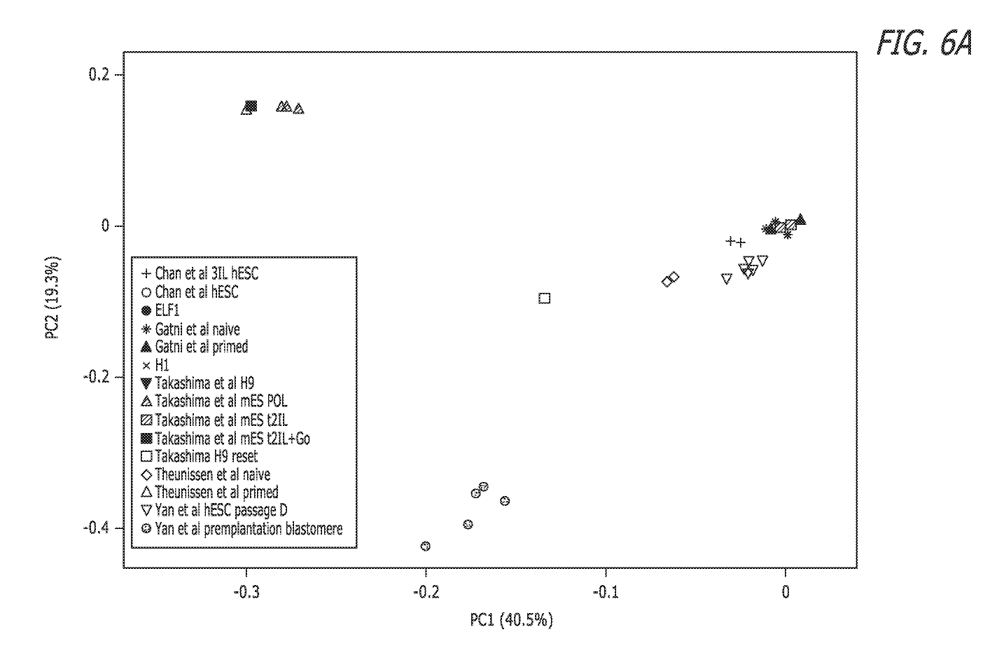

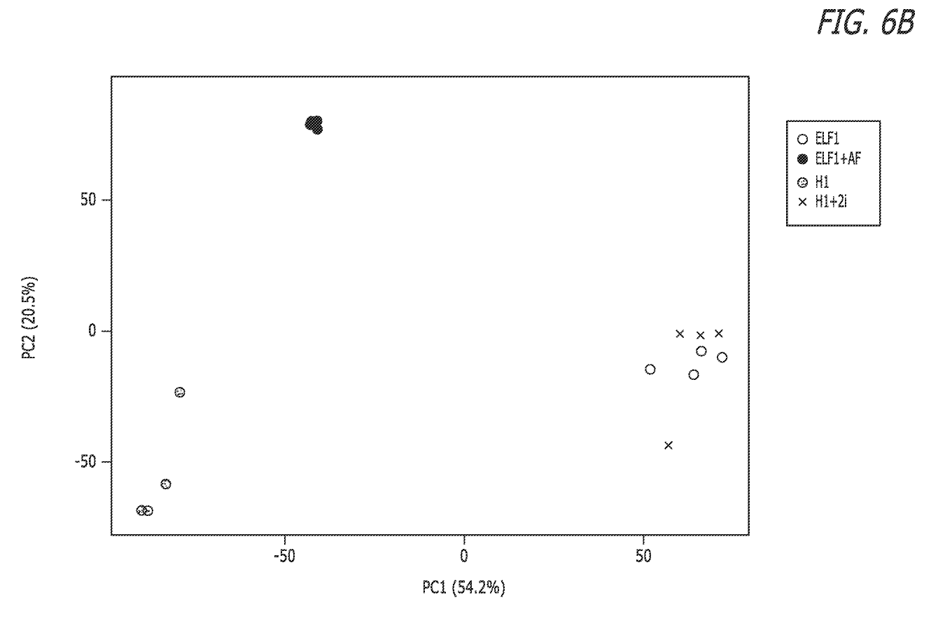

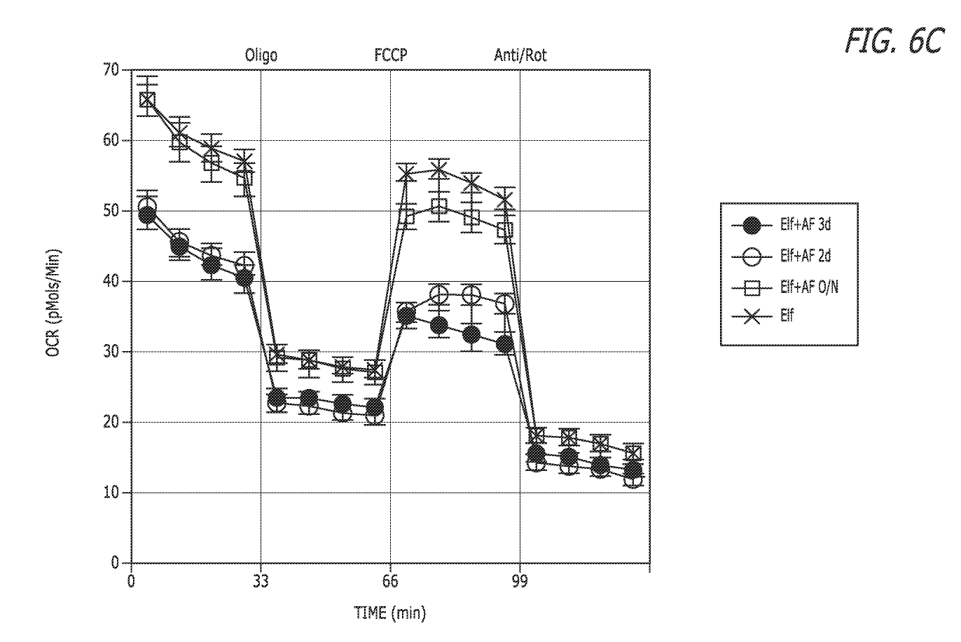

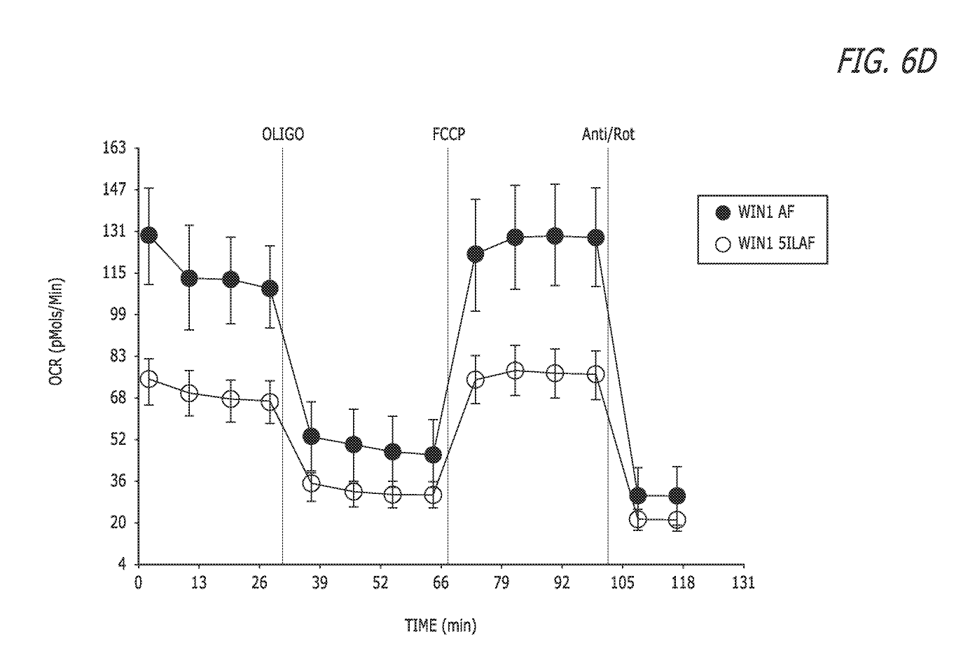

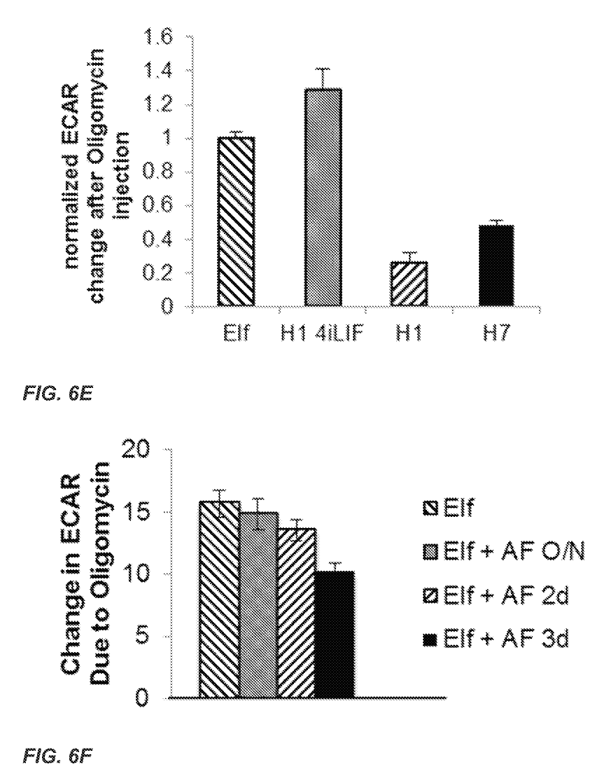

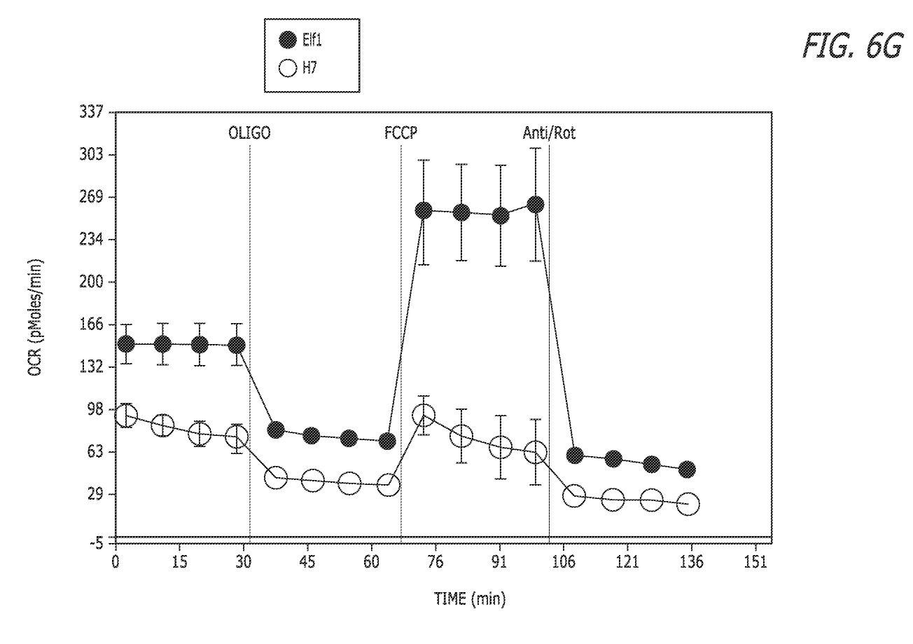

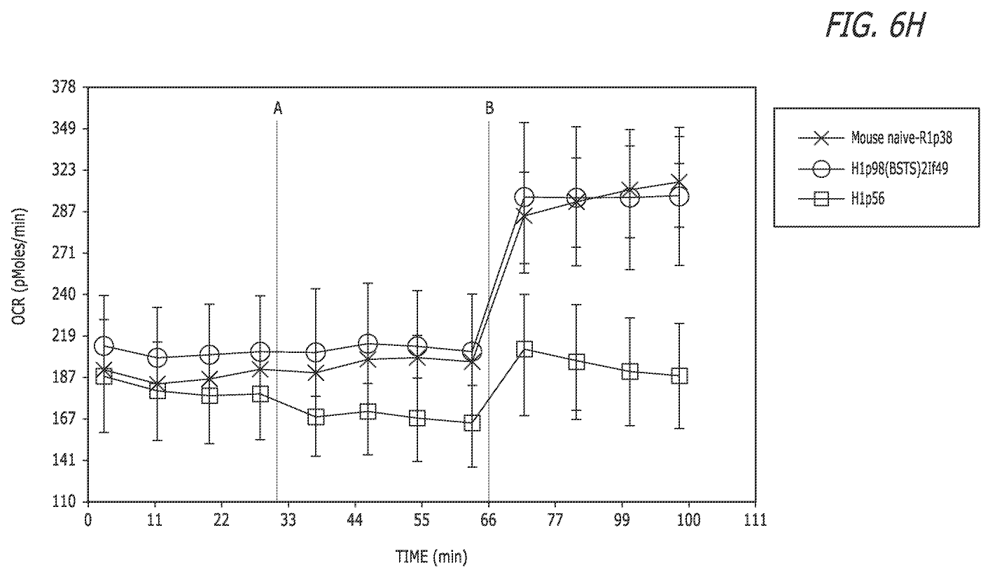

A dramatic metabolic switch occurs in mouse ESCs between pre-implantation (naïve) and post-implantation (primed) state. Human naïve counterpart has been recently toggled or derived from embryos. Principal component analysis (PCA) of the expression signatures of these new cell types confirmed that all derived human naïve hESC are in a significantly earlier stage than primed hESC4 (FIG. 1A and FIG. 6A). To assess the metabolic profiles of the human naïve and primed hESC, the cells' oxygen consumption rates (OCR) were analyzed using a Seahorse extracellular flux analyzer. As seen previously in mouse ESCs, an increase in the oxygen consumption rate was detected after FCCP addition to the newly-derived naïve hESCs (FIG. 1A, Elf18; WIN-16) while little increase was observed in primed hESCs (H1, H7) or cells transitioning to primed state (Elf1 AF, WIN1 AF) (FIG. 1B-1E, FIG. 6C-6G). Likewise, cells “toggled” back to a more naïve state using GSK3 and MEK inhibitors plus bFGF (H1 2iF8) or GSK3, MEK, JNK and p38 inhibitors in addition to LIF, IGF and FGF (H1 4iLIF) showed increased OCR in response to FCCP to a level similar to mESCs (FIG. 1B-1C, FIG. 6H-6I). These results indicate that the primed hESCs have a lower mitochondrial respiration capacity than naïve hESCs.

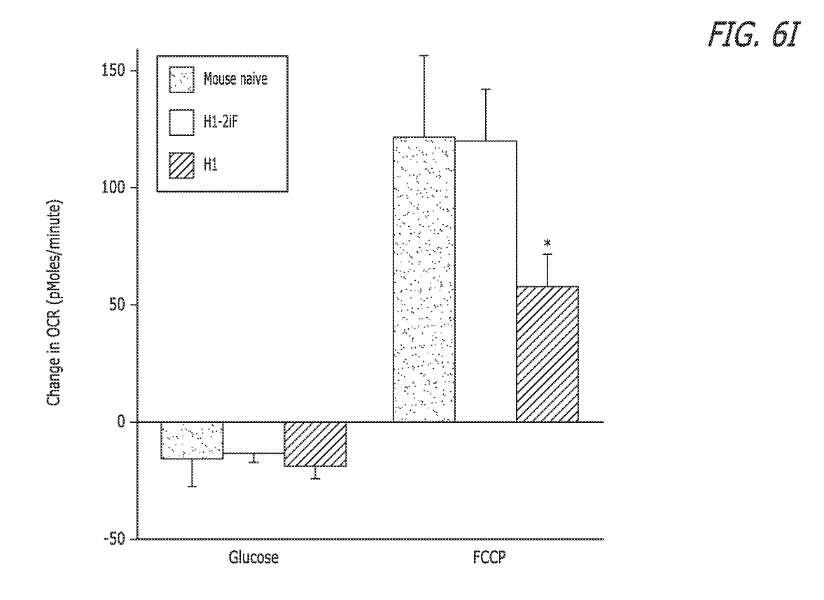

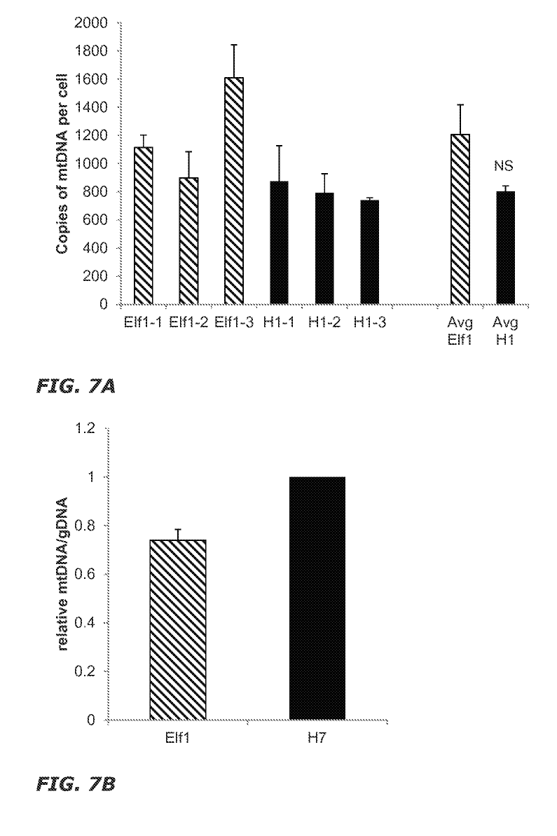

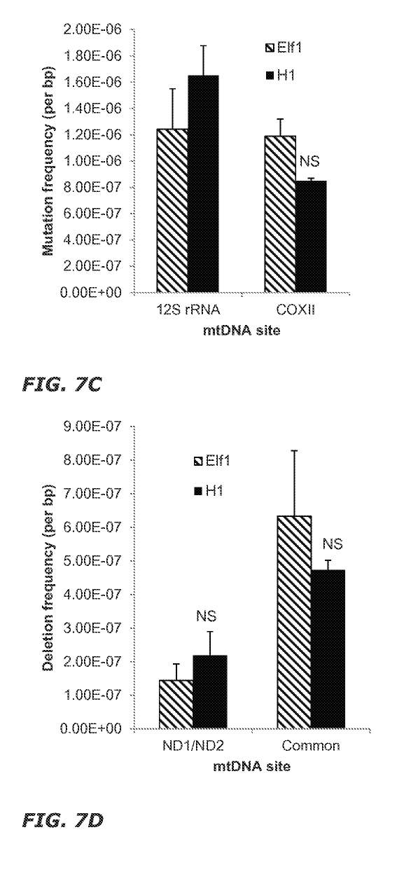

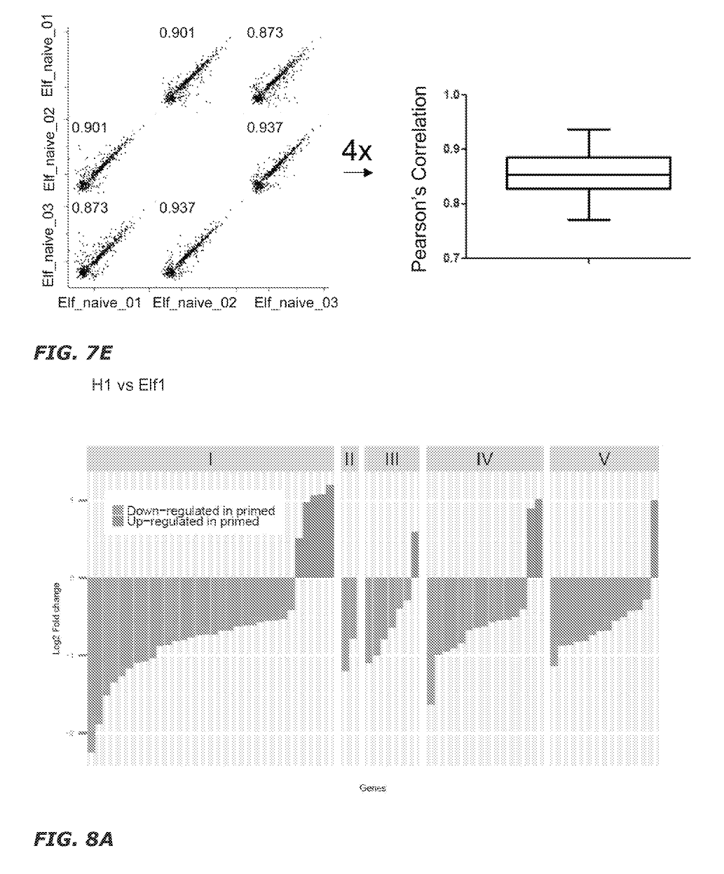

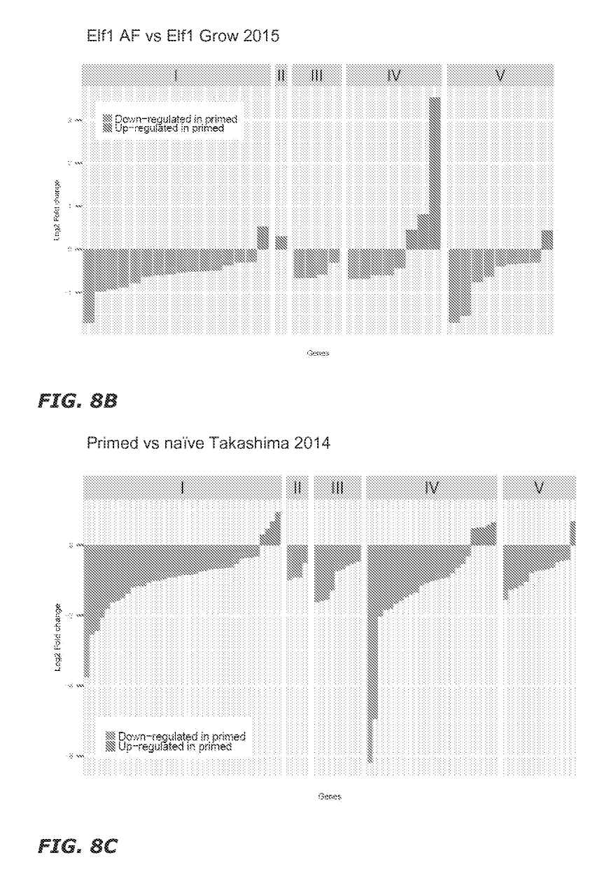

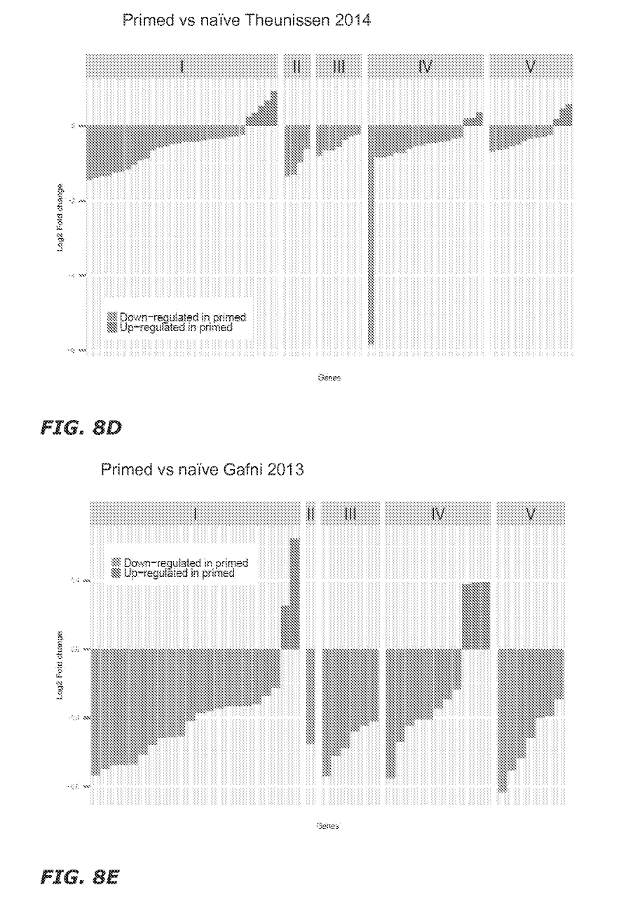

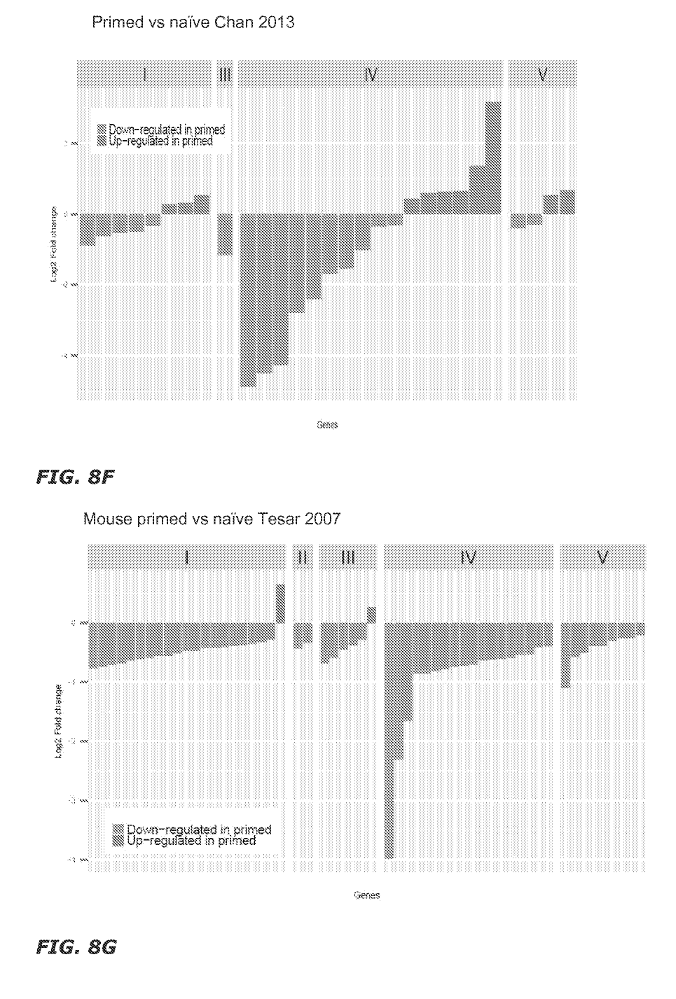

The higher mitochondrial capacity of naïve hESCs (Elf1) reflects neither more mature mitochondria, nor an increase in mitochondrial DNA (mtDNA) copy number compared to primed hESCs (EIfAF, H7, H1) (FIG. 1F, FIG. 7A-7B). Further, no obvious increase in mtDNA mutation frequency was detected in primed compared to naïve hESCs (FIG. 1G, FIG. 7C-7D), suggesting that reduction of oxidative respiration in primed hESCs is not caused by a deteriorating mitochondrial genome. However, consistent with the mouse data, RNA-seq data from this study and microarray or RNA-seq data from others showed that expression of most mitochondrial electron transport chain complex IV-cytochrome c oxidase (COX) genes is significantly downregulated in the primed state compared to the naïve state (FIG. 1H; FIG. 8A-8G). Interestingly, while COX genes are dramatically downregulated in primed hESCs, they are only slightly downregulated in Elf1 AF cells (FIG. 8B). Since Elf1 AF cells (after 3 day transition) already showed a dramatic metabolic change in Seahorse flux analyzer (FIG. 1D), changes in a potential primary controller of the electron transport chain were investigated. One such candidate is HIGD1A, the protein required for electron transport chain supercomplex formation. Importantly, primed hESCs have lower levels of HIGD1A than all published naïve hESC lines (FIG. 1I). Further PCA analysis confirmed that Elf1 AF cells are in the early process of primed transition and the early events encompass a metabolic switch (FIG. 6B). Also, consistent with the mouse data, HIF1a is stabilized in primed, but not in naïve, hESCs (FIG. 1J), correlating with a significant change in expression of prolyl hydroxylase domain-containing protein 2, PHD2 (EGLN1), the primary regulator of HIF1a steady state levels. Further support for HIF1a stabilization and activity at the primed state comes from proteomic analysis revealing a significant increase in the protein expression of HIF targets, lactate dehydrogenase A (LDHA) and Jarid2 (jumonji, AT rich interactive domain 2) at primed hESC state (Elf1 AF cells compared to Elf1 cells; FIG. 1K-1L, FIG. 7E, FIG. 1M).

### Example 2

### Differential Metabolites Between Naïve and Primed Embryonic Stem Cells

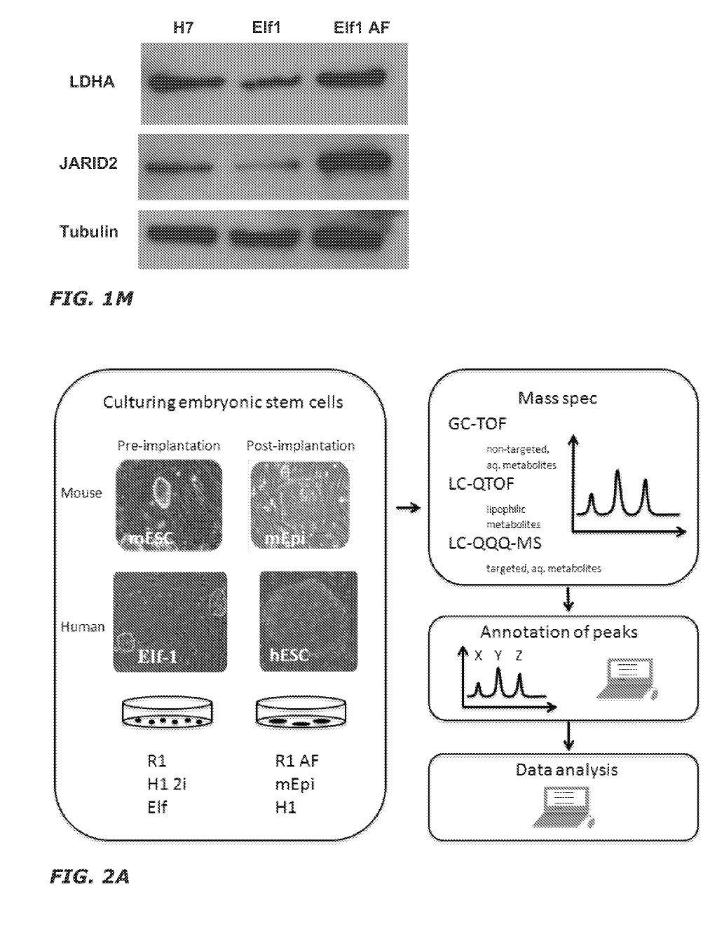

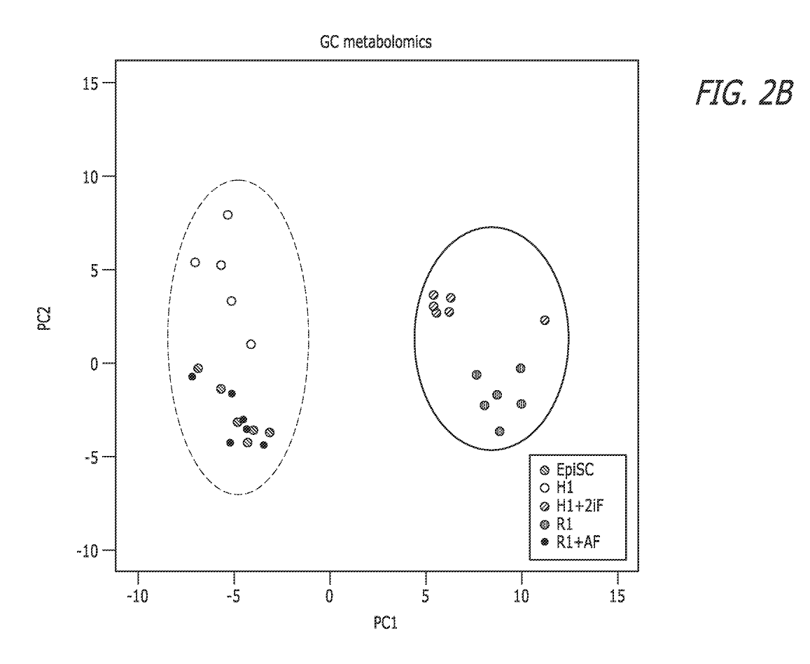

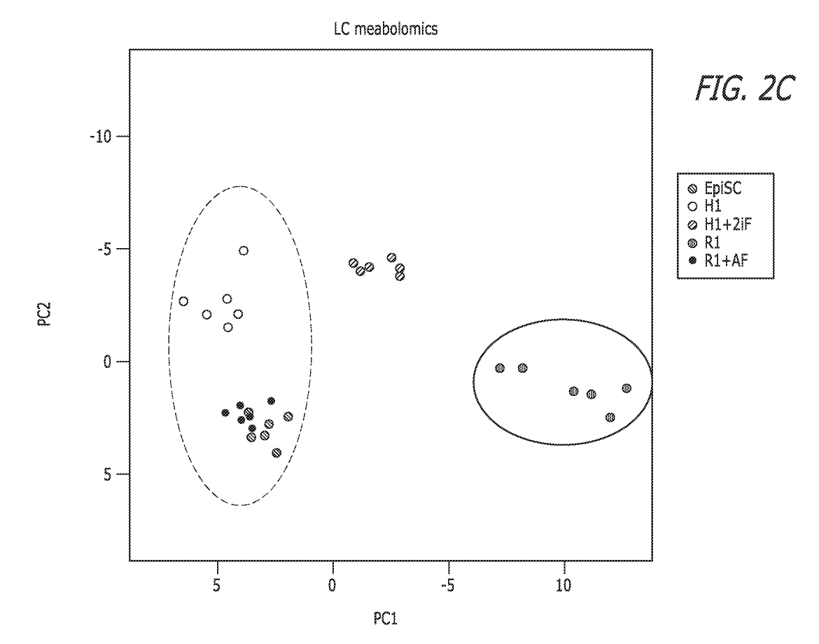

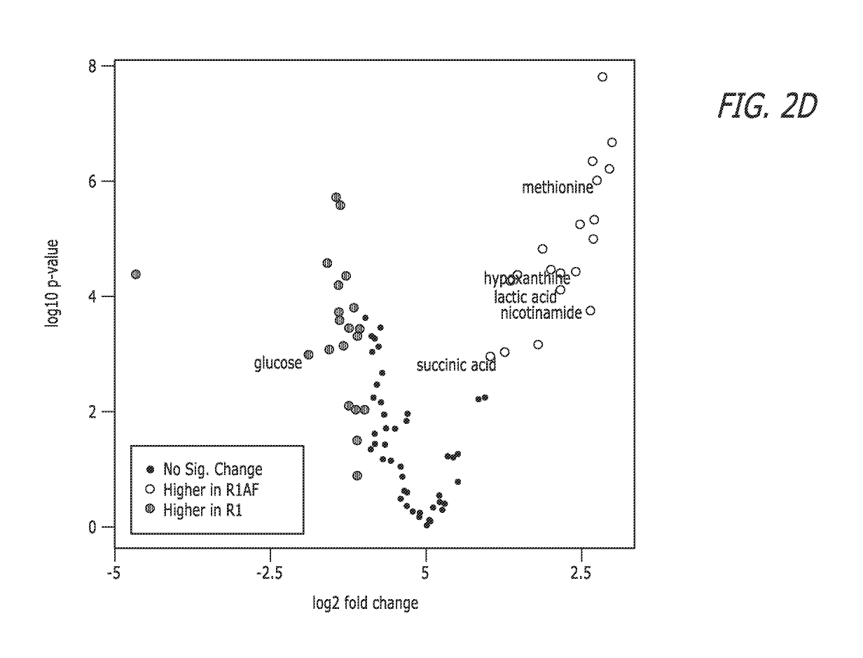

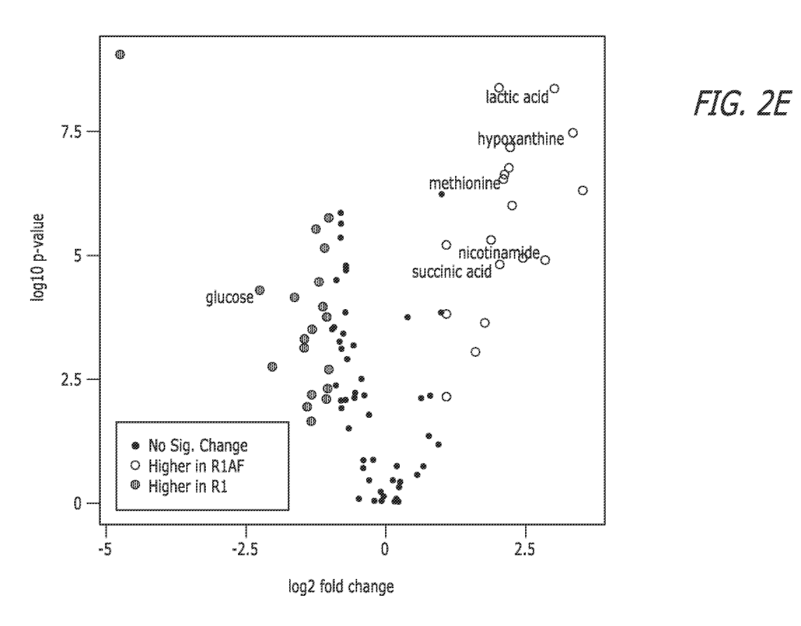

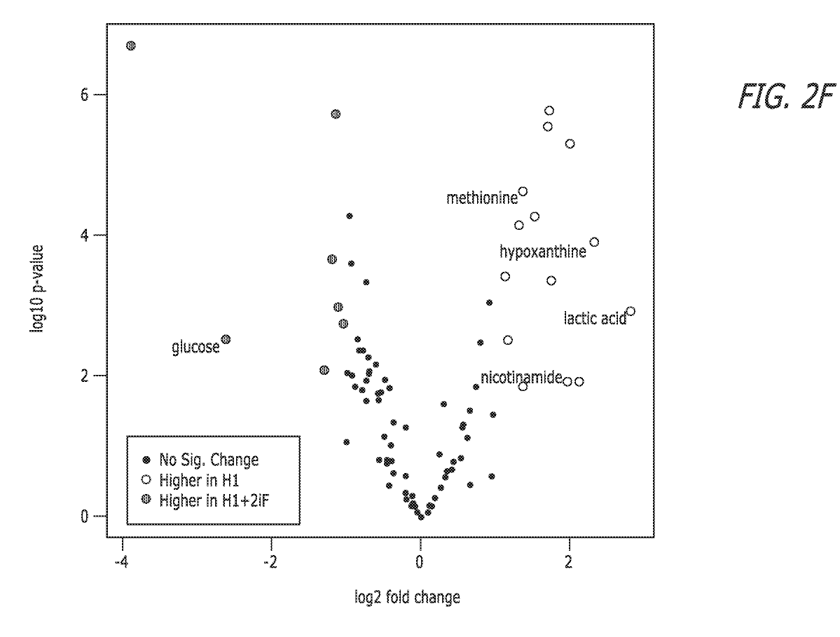

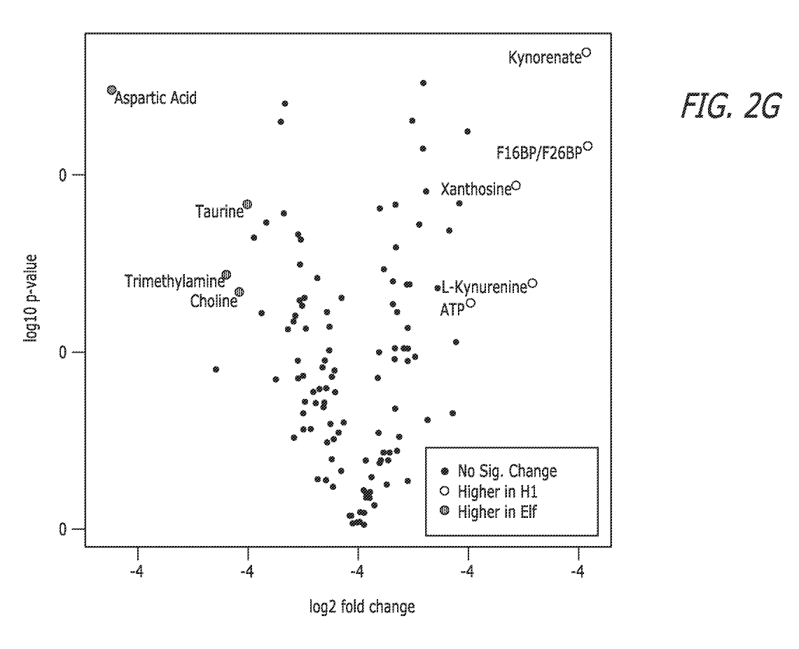

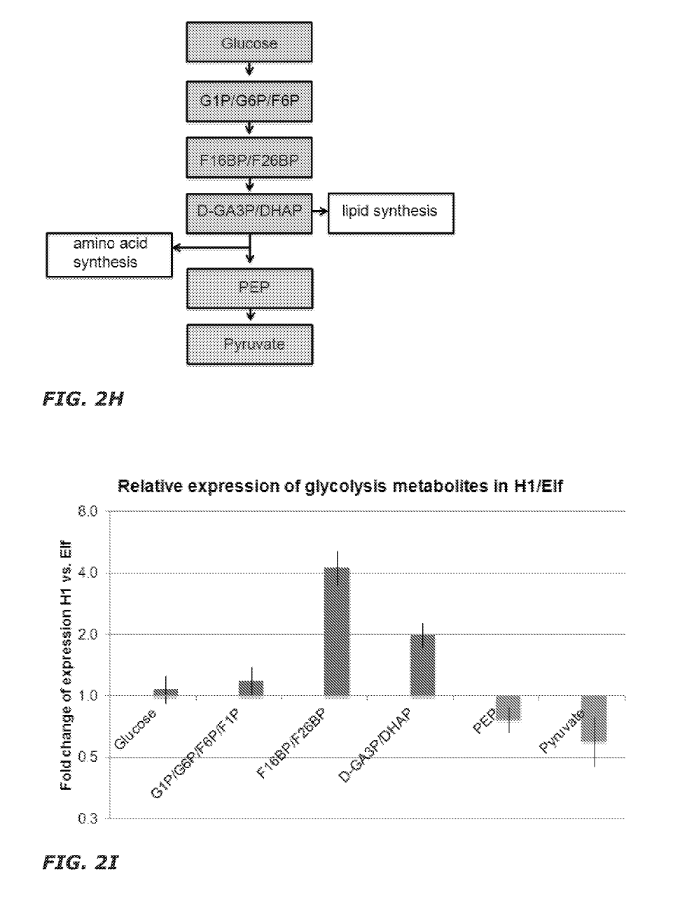

To search for critical metabolites that control the metabolic transitions between naïve and primed mouse and human ESCs, metabolic profiling was performed using non-targeted GC-TOF, LC-QTOF and targeted LC-QQQ mass spectrometry (MS) analysis. For the non-targeted MS analysis naïve and primed human and mouse ESCs were harvested using acetonitrile, sonicated and cleared by centrifugation to prepare for LC-QTOF and GC-TOF analysis. Spectral peaks were annotated using an in house annotation library and final area under the curve (AUC) values were normalized to average intensity, showing a good consistency between biological replicates as well as a clear difference in metabolite profiles between each condition (FIG. 2A).

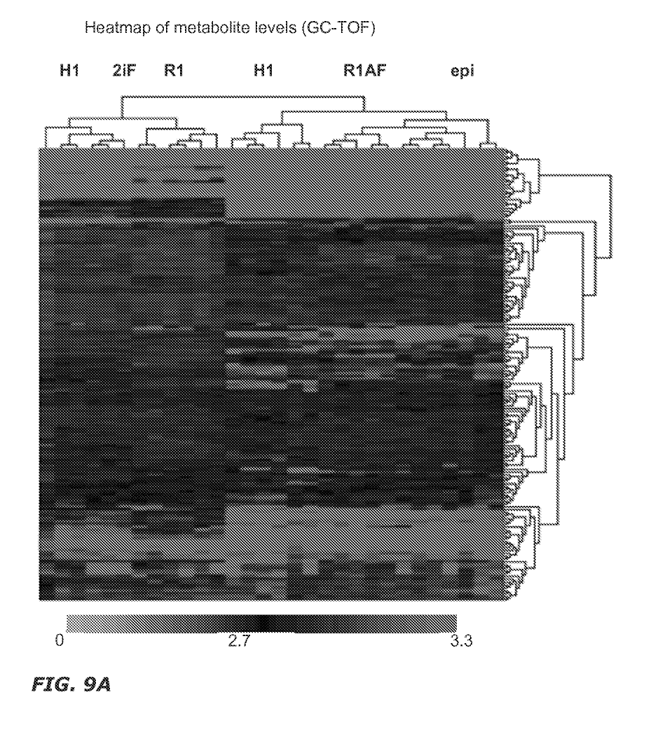

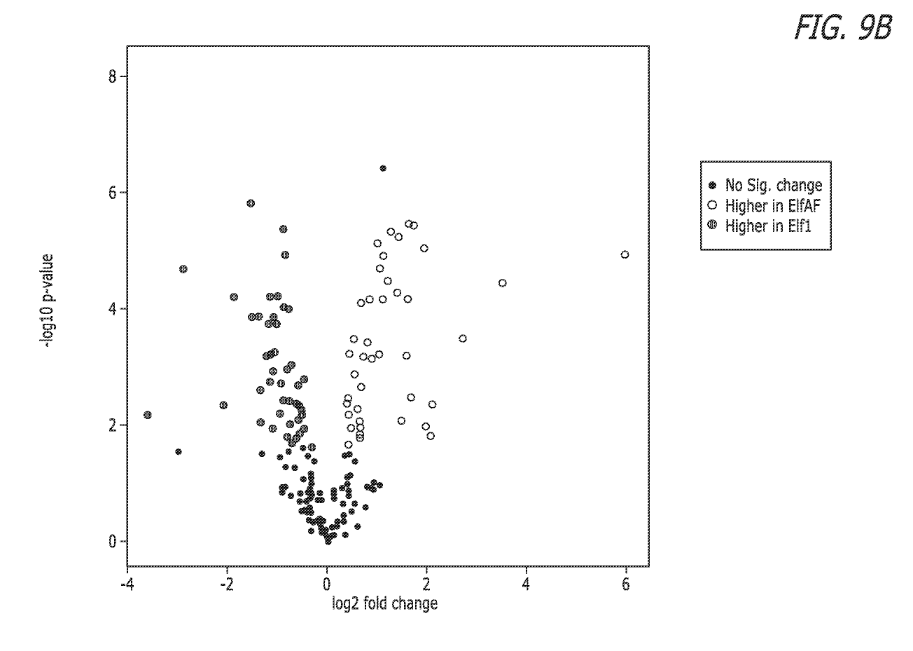

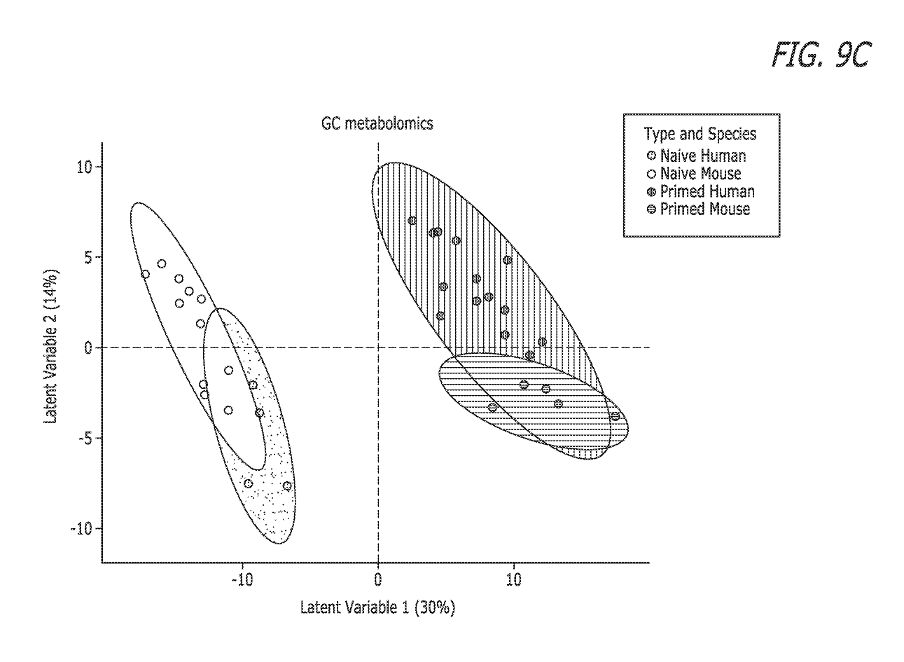

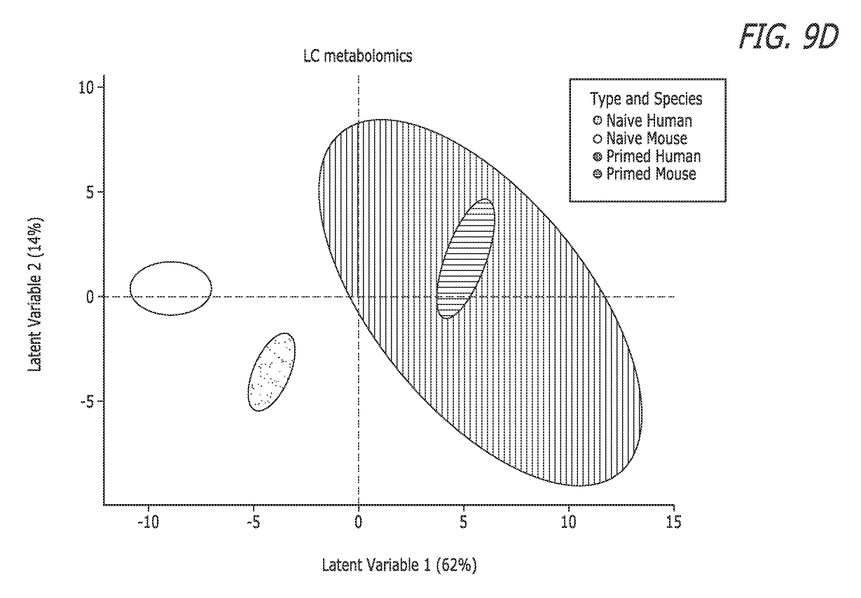

PCA reveals a difference in metabolite profiles between naïve and primed cells, regardless of species (FIG. 2B-2C, FIG. 9A-9D). Multiple naïve and primed cell lines from human and mouse separated clearly by naïve vs. primed state based on the PCA plot of GC metabolomics data (FIG. 2B; FIG. 9A-9D). The first PC, which represents the separation of naïve vs. primed cell lines, explains 50.2% of the variance, whereas the second PC explains 14.5% (FIG. 2B). Stearic acid and cholesterol are the metabolites that contribute the most to the separation within the first PC, which indicates that when ESCs transition from naïve to primed state, a major switch occurs in the lipid metabolism. A similar trend of naïve and primed ESC separation is observed in PCA plots of the LC metabolomics data (FIG. 2C, FIG. 9D). Interestingly though, H1 2iF, which is a primed cell line “toggled” towards the naïve state, and Elf1, the naïve hESC line, clustered midway between mouse naïve cell line (R1) and primed cell lines (R1AF, EpiSC, Elf1AF, H7 and H1) in LC analysis, suggesting that H1 2iF and Elf1 have not reached the same naïve state as observed in mouse, with respect to lipid signature (FIG. 2C; FIG. 9D).

Based on the GC-MS analysis a higher level of succinate was identified in the mouse primed state compared to naïve (FIG. 2D-2E). Succinate can act as an inhibitor for prolyl hydroxylase, thereby allowing HIF stabilization in the primed state. Hence succinate upregulation may be critical for primed state acquisition since HIF is stabilized in the primed state and this stabilization is shown to be sufficient to drive naïve to primed transition in mouse ESC (FIG. 1J).

In addition, targeted analysis of metabolites was performed using LC-QQQ-MS with Elf1 and H1 hESCs. Metabolites upregulated in the primed state include fructose (1,6/2,6)-bisphosphate (FI6BP/F26BP), lactate, methionine, nicotinamide and kynurenine (FIG. 2F-2G). Fold change analysis of glycolysis metabolites detected by targeted LC-QQQ-MS shows an increase in primed H1 hESCs for metabolites in the early but not late steps of glycolysis relative to naïve Elf1 hESCs (FIG. 2H-2I). Upregulation of F16BP is in concord with highly active glycolysis, however, phosphoenolpyruvate (PEP), a downstream metabolite of F16BP, does not increase in primed hESCs (FIG. 2I). Intermediates prior to PEP can be conserved for biosynthetic purposes: 3-phosphoglycerate (3PG) can be diverted to serine and glycine synthesis, which can supply one-carbon units to multiple methylation reactions (e.g., regeneration of methionine from homocysteine in the SAM cycle); dihydroxyacetone phosphate (DHAP) can be converted to glycerol, which serves as the backbone of glycerolipids. Therefore changes in lipid/fatty acid metabolism and amino acid pathways were tested (FIG. 2H).

### Example 3

### Differential Fatty Acid Metabolism in Naïve and Primed ESCs

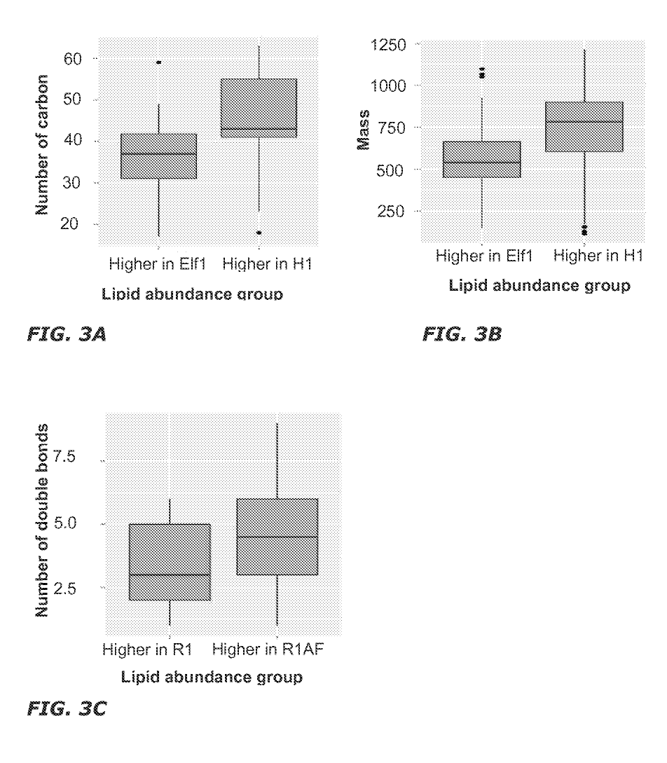

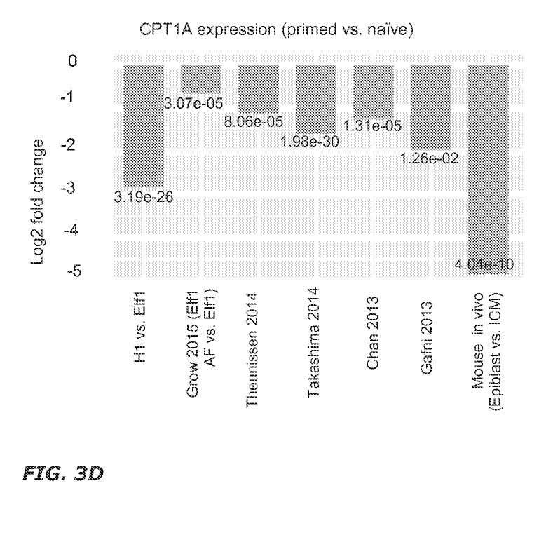

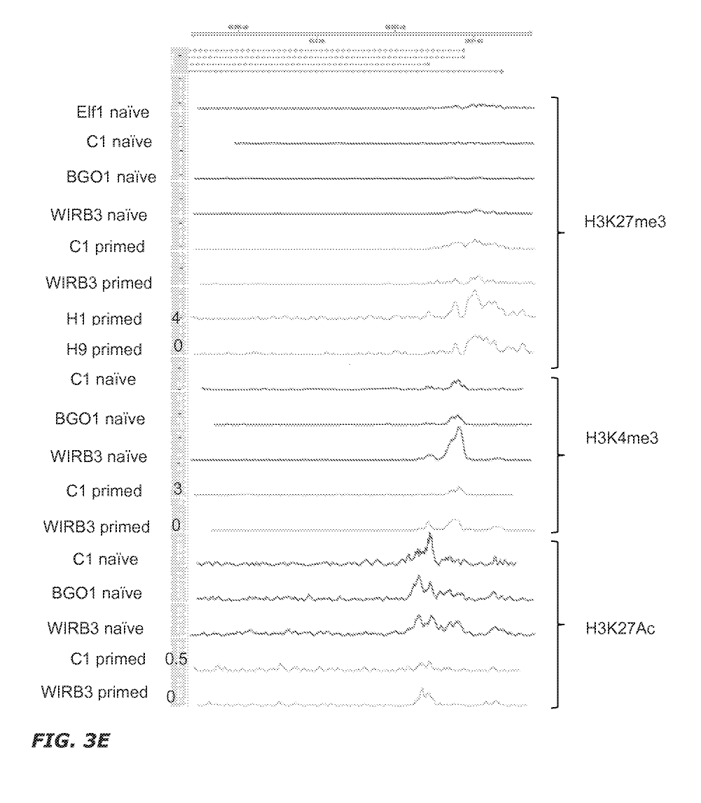

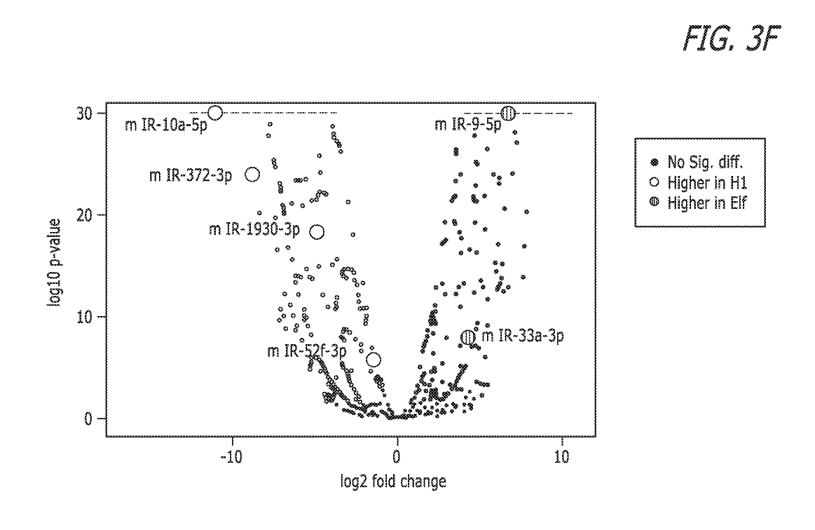

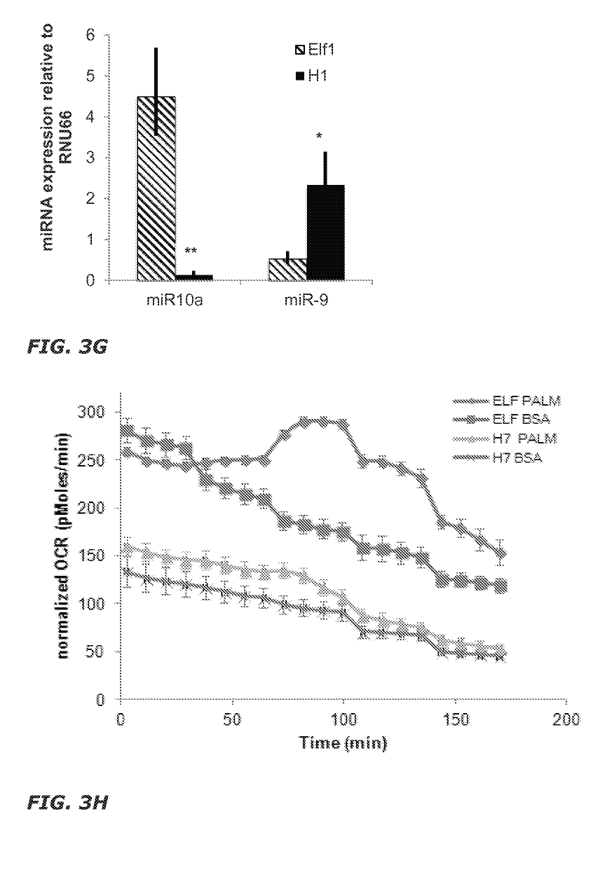

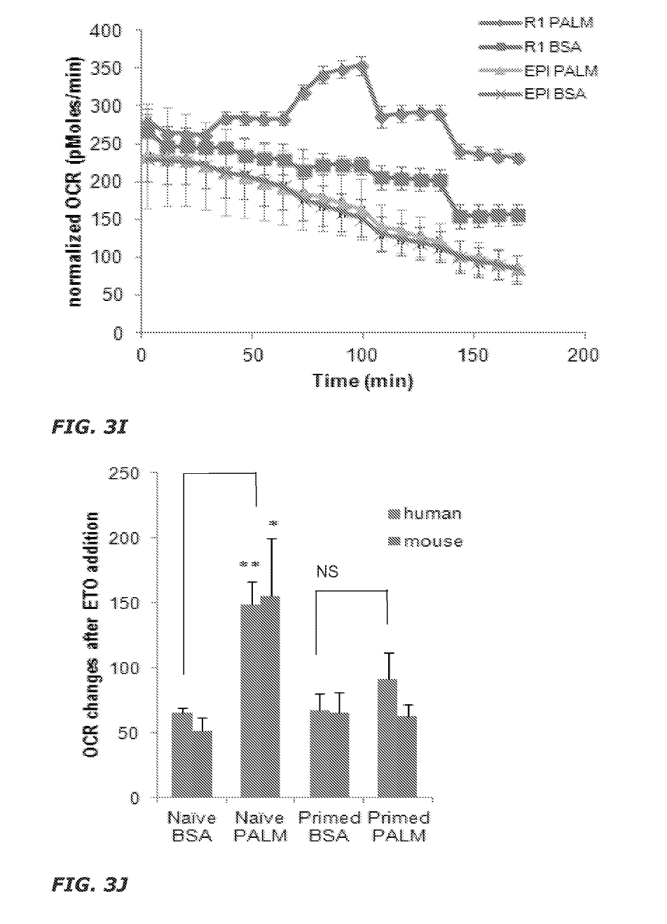

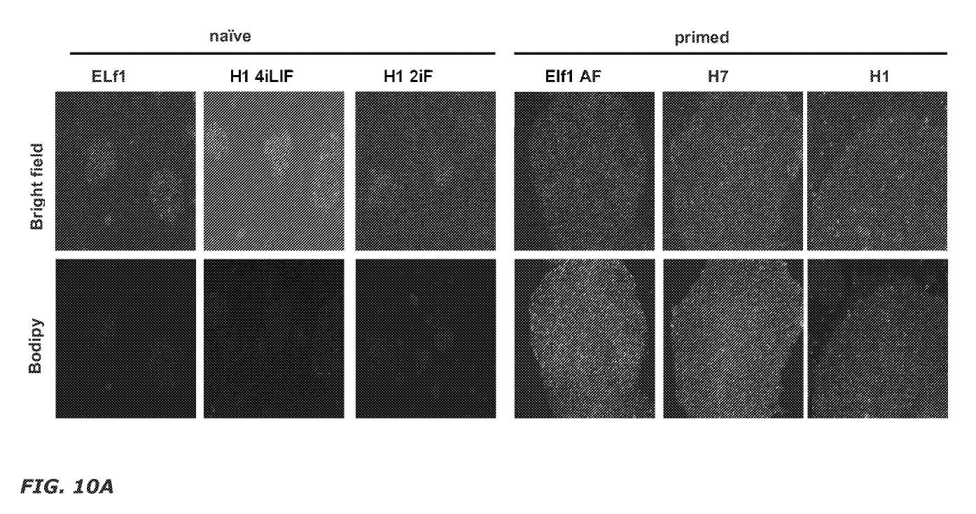

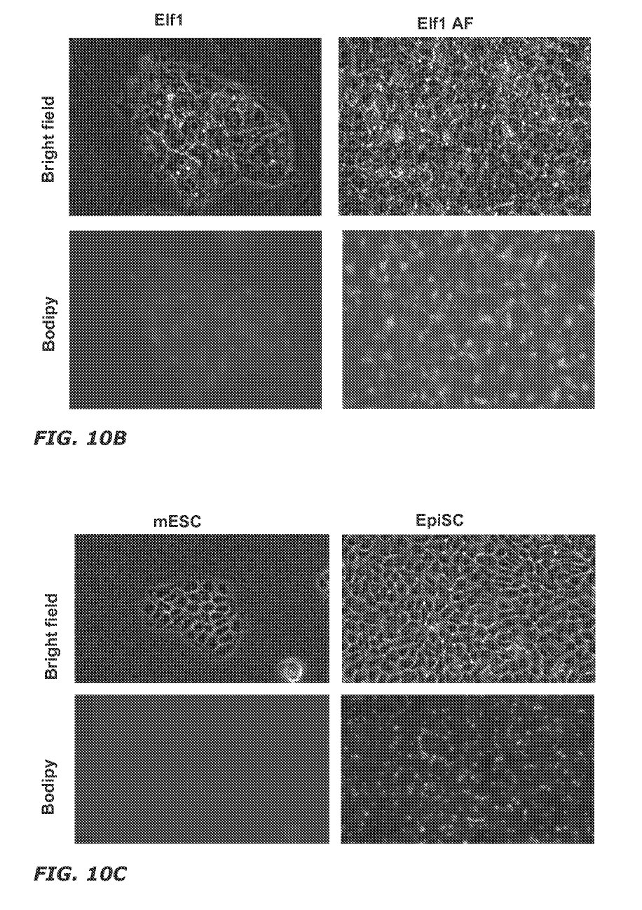

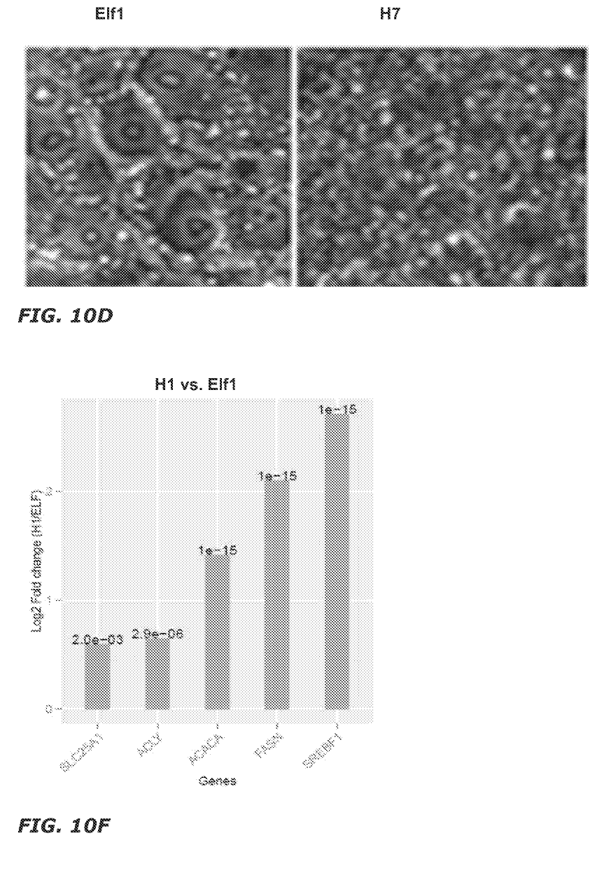

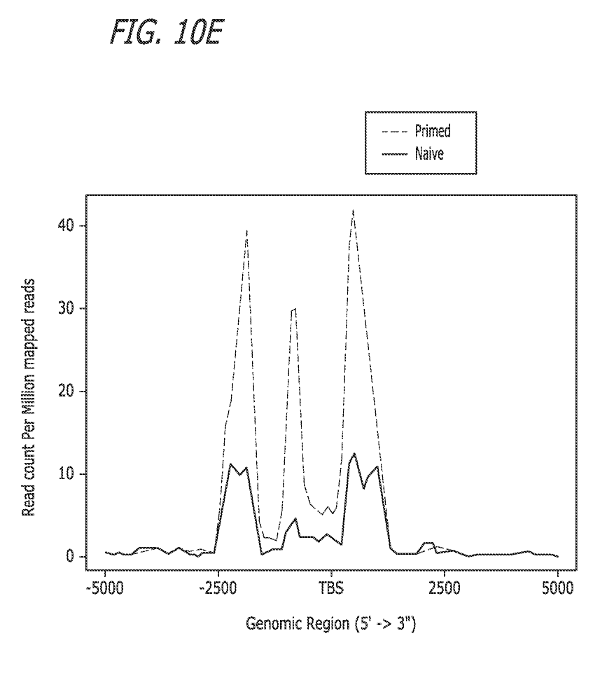

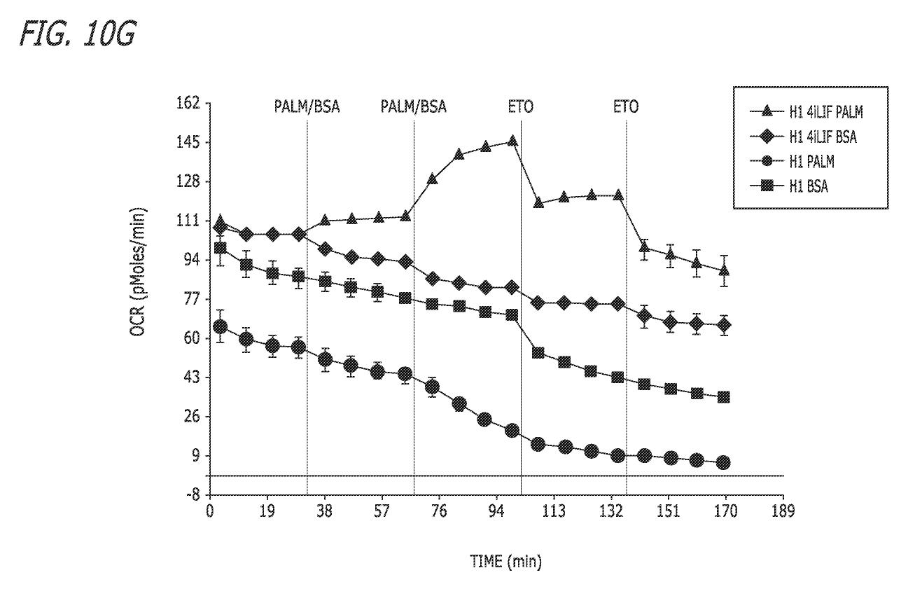

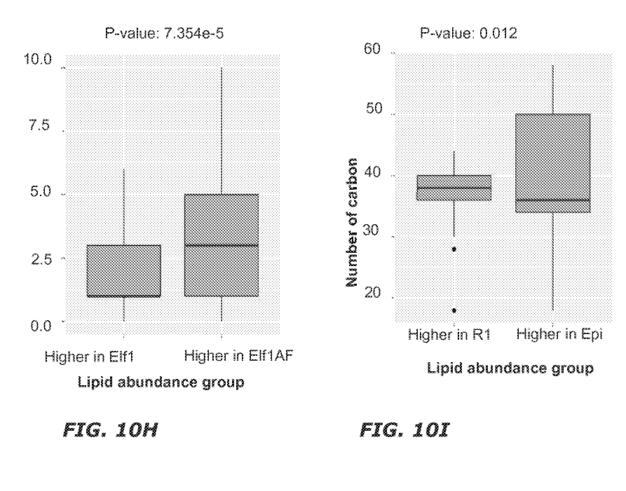

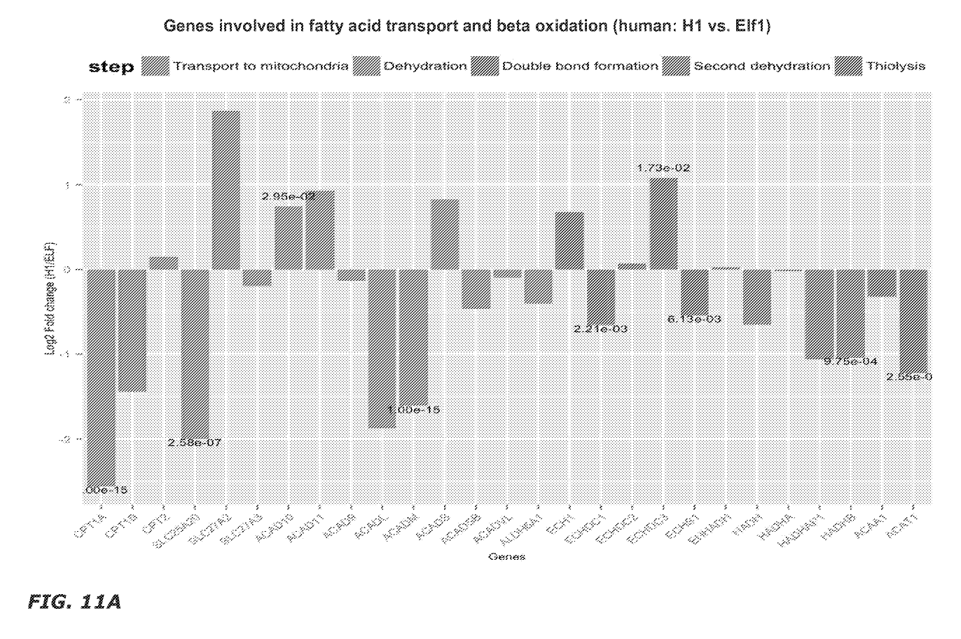

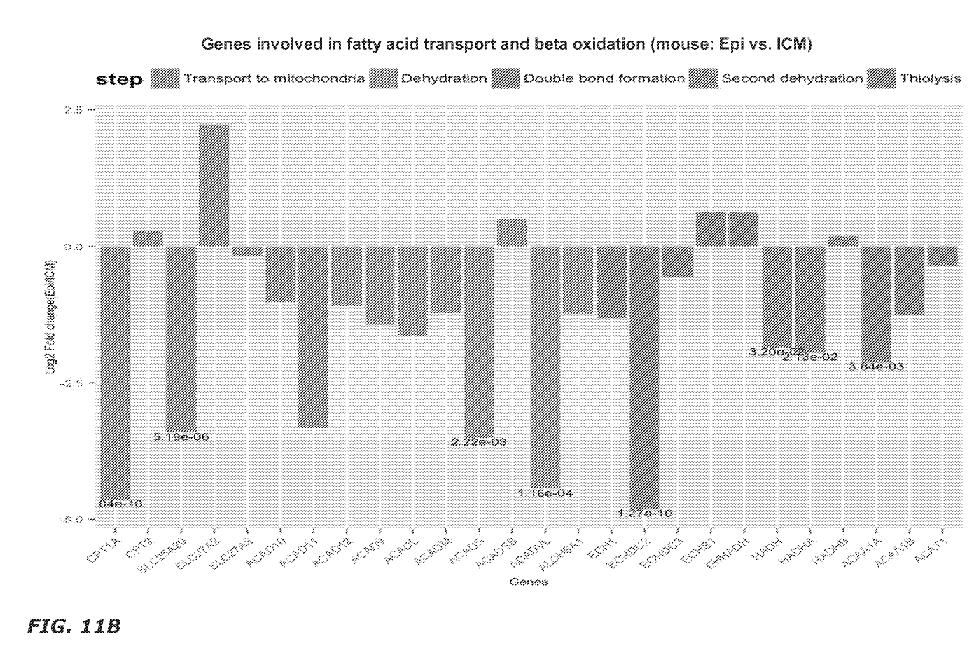

Further lipid analysis was performed using a LC-QTOF instrument on naïve Elf1 and primed H1 cells as well as non-targeted LC-QTOF analysis on Elf1, Elf AF, R1 and mEpi ESCs. All lipidomic features were classified into two groups: more abundant in H1 or more abundant in Elf1. For 119 features with identified molecular formulae and structures, lipids more abundant in H1 have higher numbers of carbons (Wilcoxon rank sum test p-value 4.30e-4, FIG. 3A). For 320 features with just identified mass, lipids more abundant in H1 are heavier (p-value=1.66e-10, FIG. 3B). Non-targeted LC-QTOF also showed that lipids more abundant in mEpi than R1 have significantly higher number of carbons (p-value=0.012, FIG. 10I). When sorting based on level of unsaturation lipids more abundant in primed R1AF have a higher number of double bonds than lipids more abundant in naïve R1 (p-value=0.044, FIG. 3C), which can also be observed in primed Elf1 AF compared to naïve Elf1 (p-value=7.35e-5).

In concordance with the significant increase in long carbon chain lipids observed in primed mouse and human ESCs, a significant increase in accumulation of lipid droplets was also detected in the primed state, as observed by Oil Red 0 and BODIPY staining (FIG. 10A-10D). These data indicate an increased synthesis and/or decreased beta-oxidation in primed cells.

To identify the cause for accumulation of lipids in primed ESCs, differences among the enzymes involved in fatty acid metabolism were studied. Interestingly, several of the enzymes involved in fatty acid transport into the mitochondria and fatty acid beta-oxidation are significantly downregulated in primed human ESCs, as well as in mouse in vivo post-implantation state (FIG. 1I).

Carnitine acyltransferase 1 (CPT1) transfers long chain acyl groups to carnitine. This enzyme is responsible for a very important step of mitochondrial fatty acid beta-oxidation by facilitating the initial step in acyl transfer to the mitochondrial matrix. Three isoforms have been described: CPT1A, CPT1B and CPT1C; however, CPT1C is not involved in fatty acid beta-oxidation. Interestingly, the rate limiting fatty acid transporter CPT1A is downregulated in both mouse in vivo post-implantation and human primed ESC state compared to all analyzed naïve states (FIG. 3D). The decrease in CPT1A expression in the primed state could be due to increased H3K27me3 and decreased H3K4me3 and H3K27ac marks observed in CPT1A promoter in primed hESCs (FIG. 3E; FIG. 10E). miRNA analysis of Elf1 compared to primed HI cells reveals that several of the miRNAs predicted to target CPT1A and other enzymes involved in beta-oxidation are up-regulated in primed hESCs (e.g., miR-9, miR-33a-3p, FIG. 3F). Moreover, microRNAs predicted by Targetscan and miRTarBase to target enzymes involved in fatty acid synthesis were downregulated in primed cells (e.g., miR-10a and miR-193, FIG. 3F). Concomitantly, key fatty acid synthesis genes were up in primed H1 hESCs compared to naïve Elf1 state (SLC25A1, ACLY, ACACA, FASN, and SREBP-1c; FIG. 10F). miRNAs were further validated by qPCR analysis and showed that miR-9, predicted to target CPT1A, was upregulated, while miR-10a, predicted to target SREBP-1c (a regulator of fatty acid and cholesterol synthesis), was downregulated in the human primed state (FIG. 3G).

To test the level of fatty acid beta-oxidation in naïve and primed human and mouse ESCs, a palmitate-oxidation assay was performed in the Seahorse metabolic flux analyzer. Importantly, both mouse and human naïve ESCs were capable of utilizing palmitate as an energy source, while primed mouse or human ESCs were not (FIG. 3H-3J; FIG. 10C). This result suggests that primed human and mouse ESCs are not capable of significant beta-oxidation and, in combination with increased fatty acid synthesis, may explain the accumulation of lipids observed in this state (FIG. 10A-10D).

### Example 4

### Differential Amino Acid Metabolism in Naïve and Primed ESCs

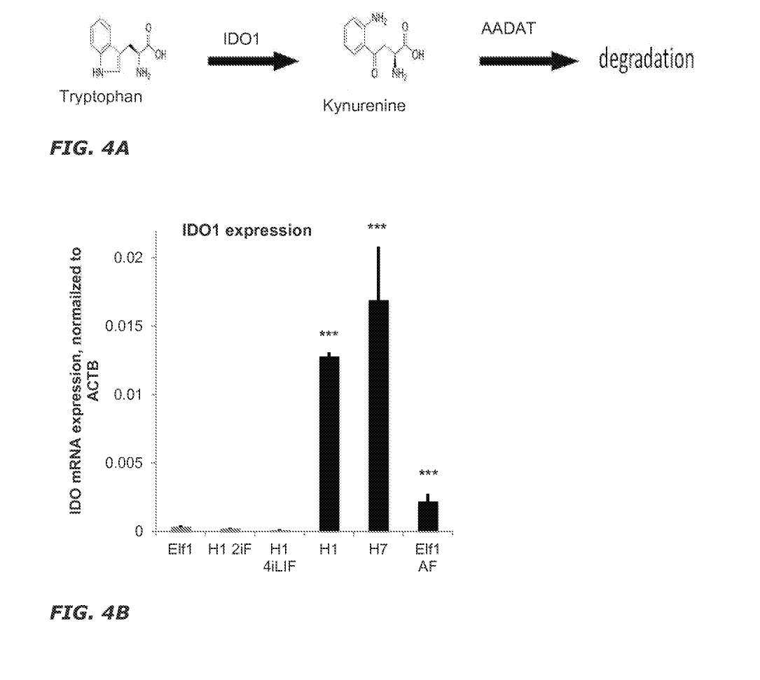

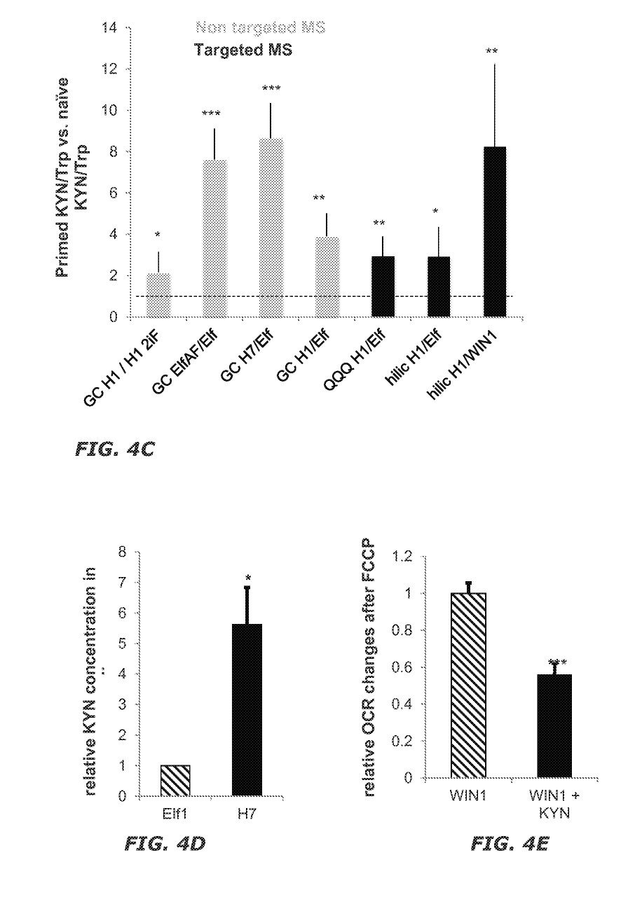

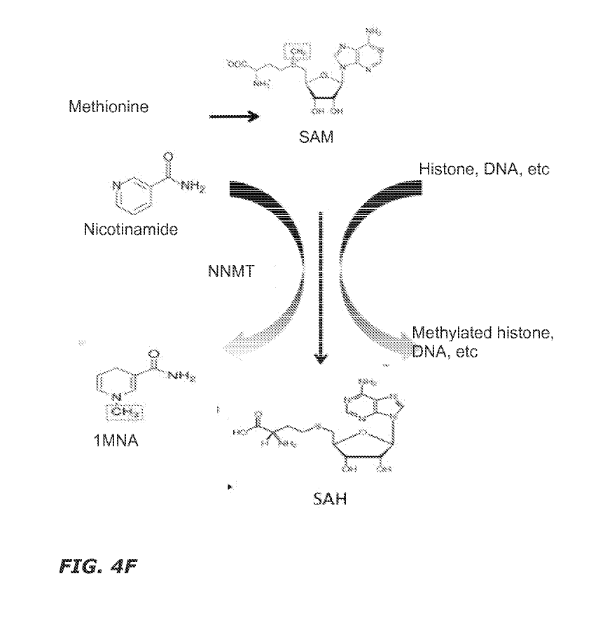

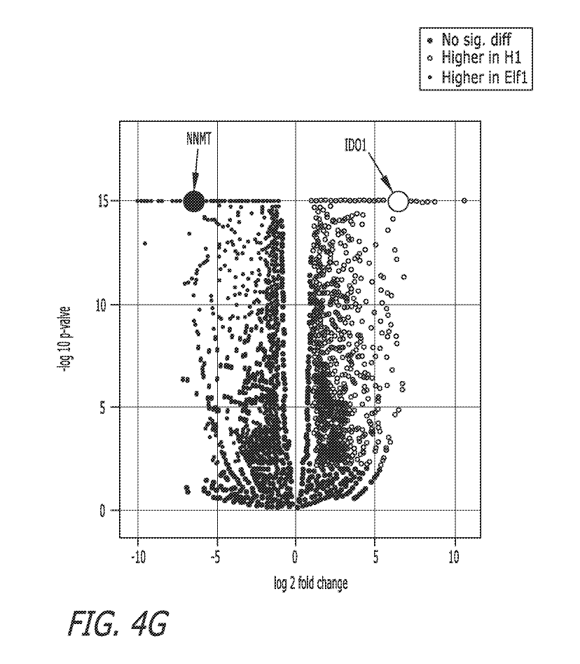

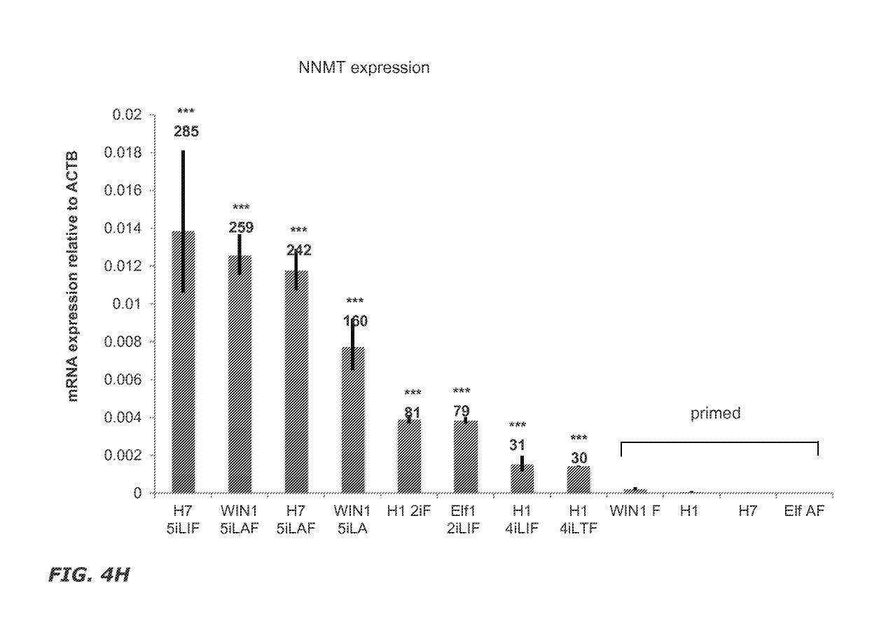

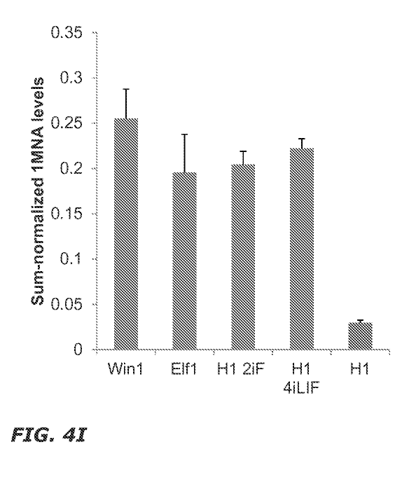

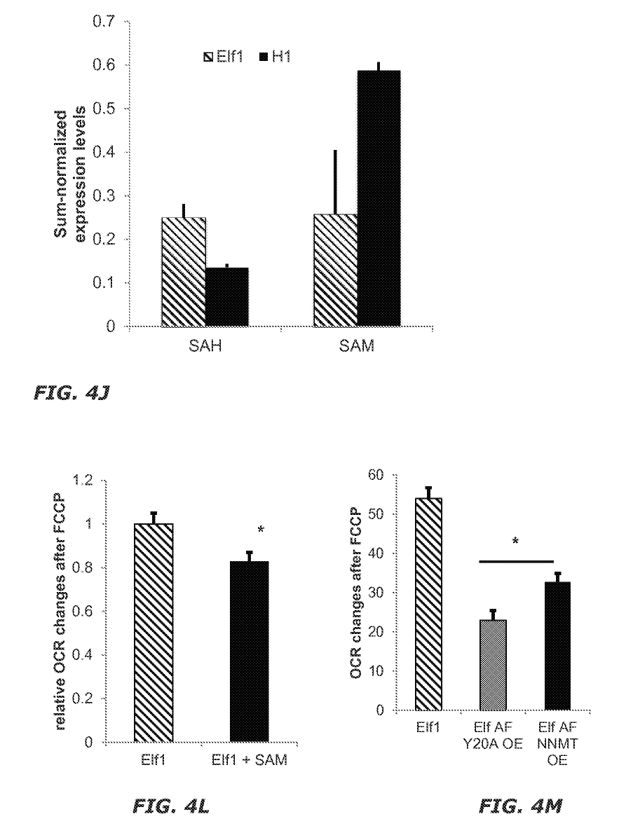

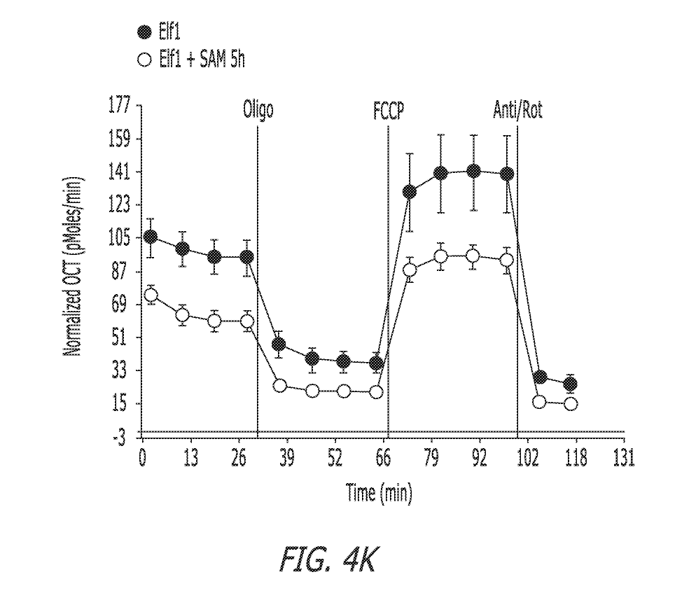

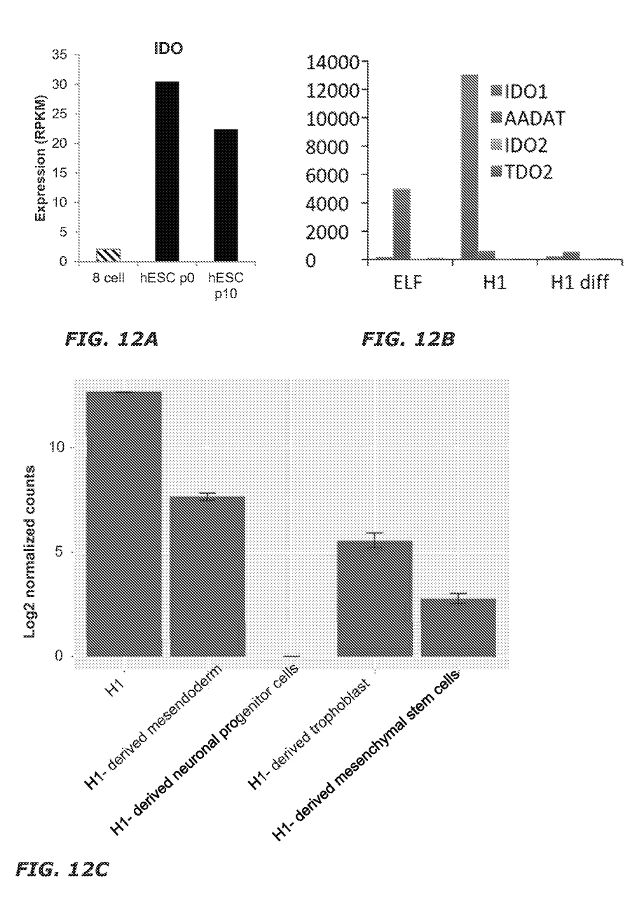

In addition to glycolysis and fatty acid metabolism, primed cells show changes in amino acid metabolism pathways. In primed vs. naïve hESCs a large enrichment of the tryptophan degradation product kynurenine was observed, which can act as a ligand for the nuclear receptor AHR29 (FIG. 4A). Interestingly, tryptophan is shown to be critical for primed hESCs growth. RNAseq and qPCR data show a large increase of the tryptophan metabolizing enzyme IDO1 in primed hESCs (FIG. 4B, 4G), providing further evidence of a major change in the tryptophan degradation pathway. IDO1 is also upregulated in primed hESCs compared to the in vivo eight cell human embryo (FIG. 12A). After peaking in primed hESCs, IDO1 levels quickly drop when the hESCs begin to differentiate, indicating that the function of IDO1 is specific for the primed state (FIG. 12B-12C). In addition to the consistent increase from the naïve to primed state in kynurenine vs. tryptophan ratios observed in intracellular metabolite levels (FIG. 4C), secreted kynurenine can be measured in the media of primed hESCs (FIG. 4D). In addition, the naïve to primed hESC metabolic switch is accelerated if kynurenine is added to the media (FIG. 4E).

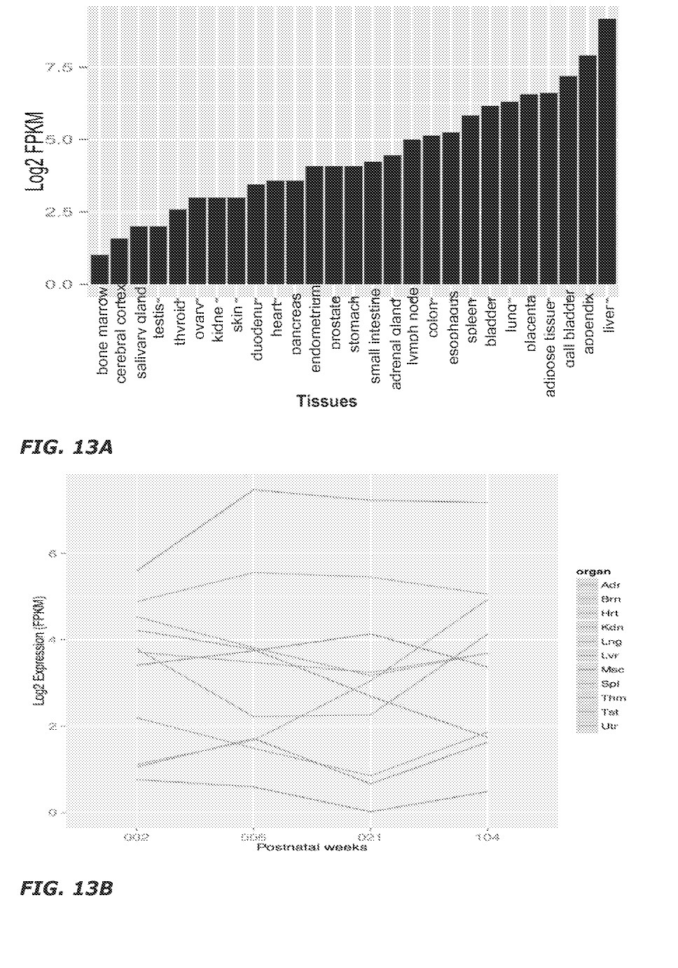

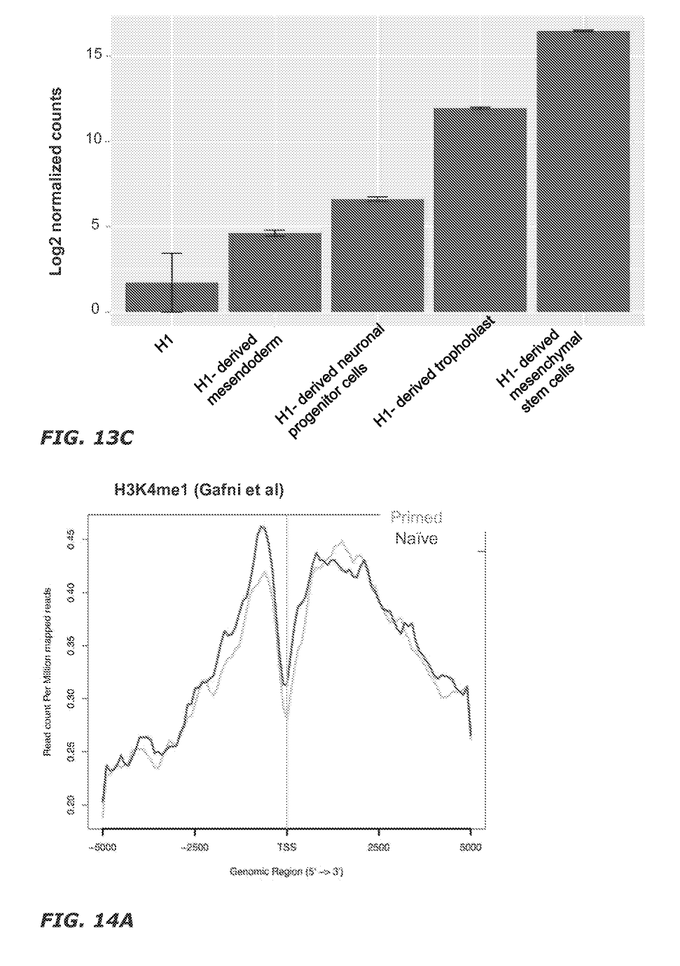

Methionine and nicotinamide downregulation along with 1-methyl-nicotinamide (1MNA) upregulation in the naïve state correlates with upregulation of nicotinamide N-methyltransferase (NNMT), shown previously to create a metabolic methyl sink, thereby promoting epigenetic remodeling in cancer (FIG. 4F-4I). Primed hESCs show an increase in SAM and a decrease in SAH levels compared to the naïve state (FIG. 4I). The increase in SAM correlates with the sharp decrease in NNMT enzyme levels observed in primed hESCs (RNA-seq, microarray and qPCR data; FIG. 4G-4H), suggesting that SAM levels may be reduced in the naïve state by high NNMT activity. Further, significant expression changes of NNMT among various tissues reveal that NNMT is dynamically regulated during development and suggest that NNMT might act as regulator of SAM levels also in a developmental context, not only in cancer (FIG. 13). Accordingly, high levels of SAM induce a primed-like metabolic profile in naïve hESCs while overexpression of NNMT, but not the mutated form of NNMT delays the naïve to primed hESC metabolic switch (FIG. 4K-4M).

### Example 5

### NNMT Regulates Repressive Histone Modifications

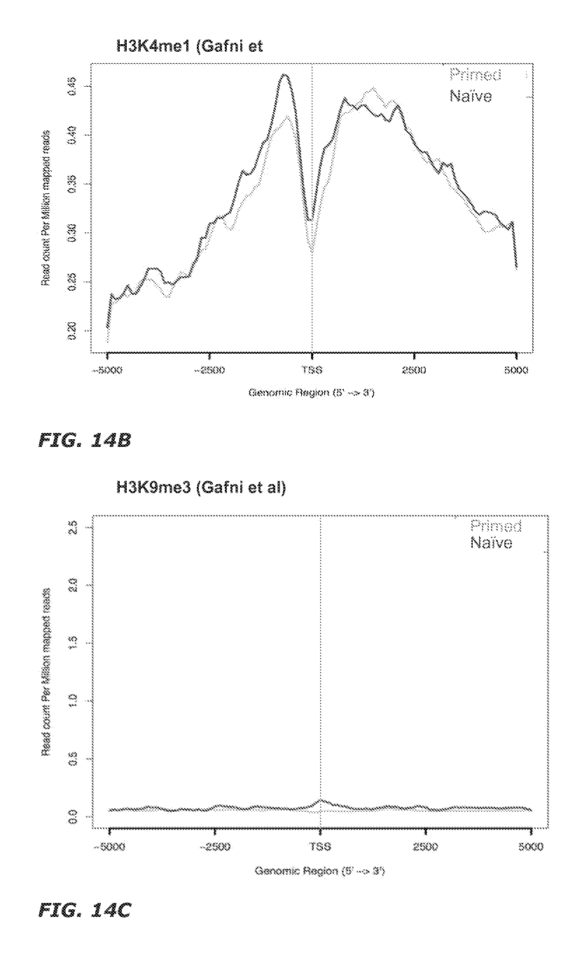

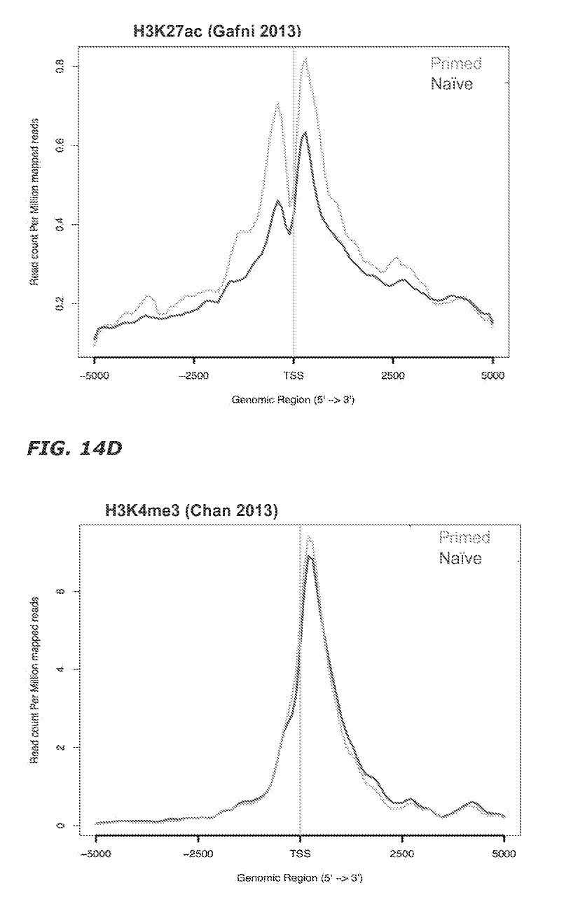

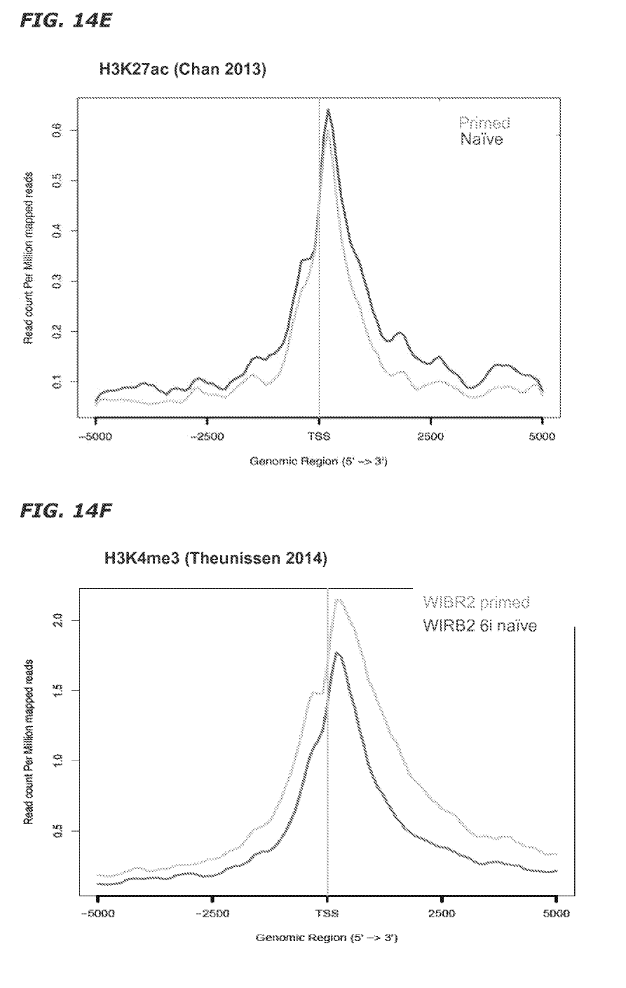

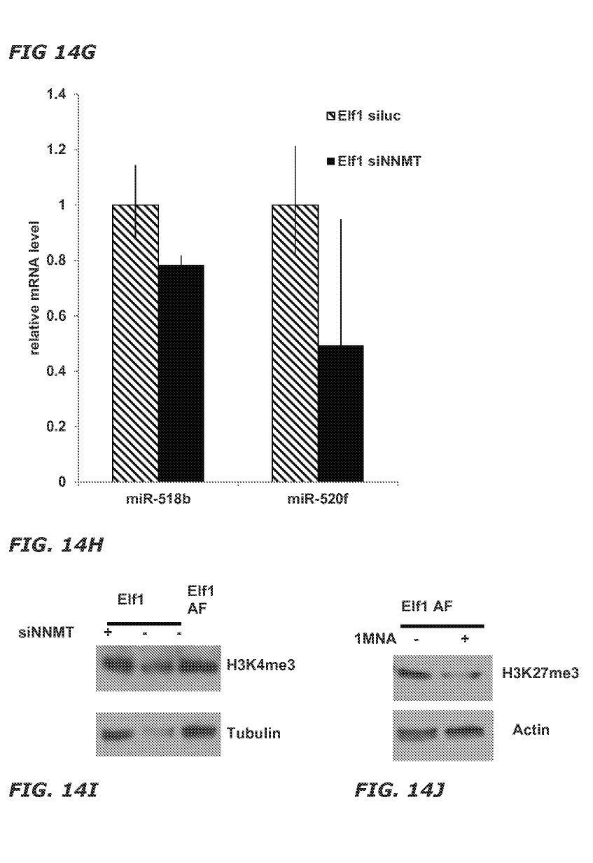

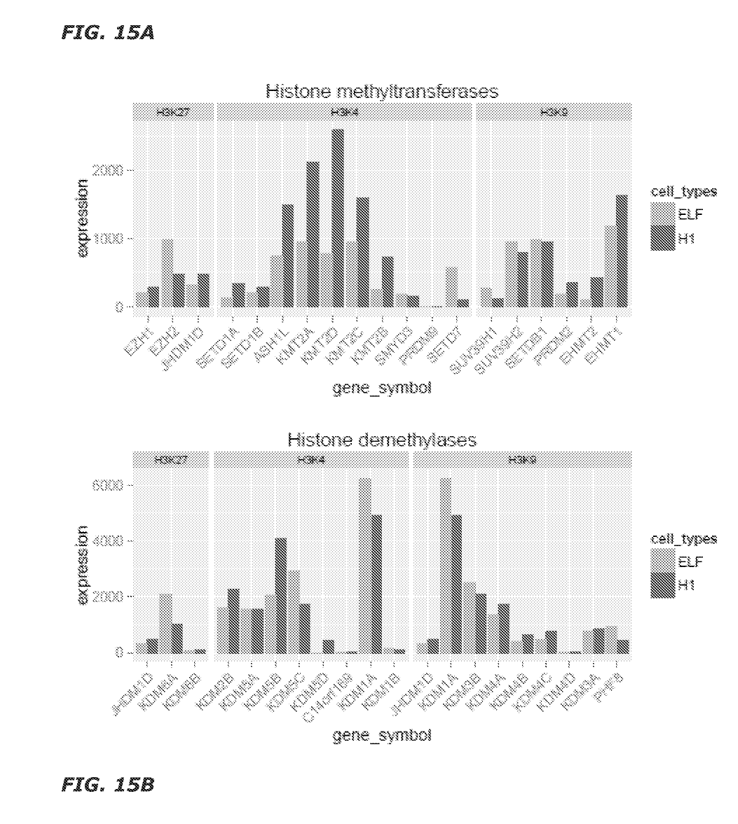

Reduction of NNMT levels during the naïve (Elf1) to primed transition correlates with a significant increase in SAM levels and in H3K27me3 histone methylation marks in 648 developmentally regulated genes (FIG. 5A). This correlation is observed in other naïve lines (FIG. 5A-5B). Moreover, Western blot analysis revealed an overall increase of H3K27me3 and H3K9me3 marks in primed hESCs (H7) compared to naïve hESCs (Elf1), while the H3K9/K14 acetylation marks remained unchanged (FIG. 5C). ChIP-seq analysis of other marks (H3K4me1, H3K4me3 and H3K27ac) did not show any significant change between primed and naïve hESCs (FIG. 14A-14G). RNA-seq analysis of histone methyltransferases and histone demethylases involved in H3K27 and H3K9 methyl marks did not show changes in expression levels that could explain the significant increase in repressive methylation marks observed at the primed state (FIG. 15A-15B). Furthermore, Western analysis showed that the protein levels of the polycomb repressive complex 2 (PRC2) regulator, EED, were not significantly changed between the two pluripotent states (FIG. 5C).

To test whether NNMT is causal for low H3K27me3 and H3K9me3 levels in naïve hESC, NNMT was knocked down in Elf1 using siRNA (80% reduction of NNMT; FIG. 5D) and analyzed the histone methylation marks. This direct reduction of NNMT levels in naïve hESCs significantly reduced the level of its product, 1MNA, reduced naïve hESC enriched microRNA miR-10a and increased H3K27me3 and H3K9me3 marks, as analyzed by Western blots, while H3K9/K14 acetylation and H3K4me3 marks did not change (FIG. 5D-5E, FIG. 14H-14I), suggesting that NNMT is a key regulator of the histone repressive methylation marks in the naïve state of pluripotency. Moreover, the addition of 1 MNA to the media of primed hESCs (Elf1 AF) decreased H3K27me3 marks (FIG. 14J).

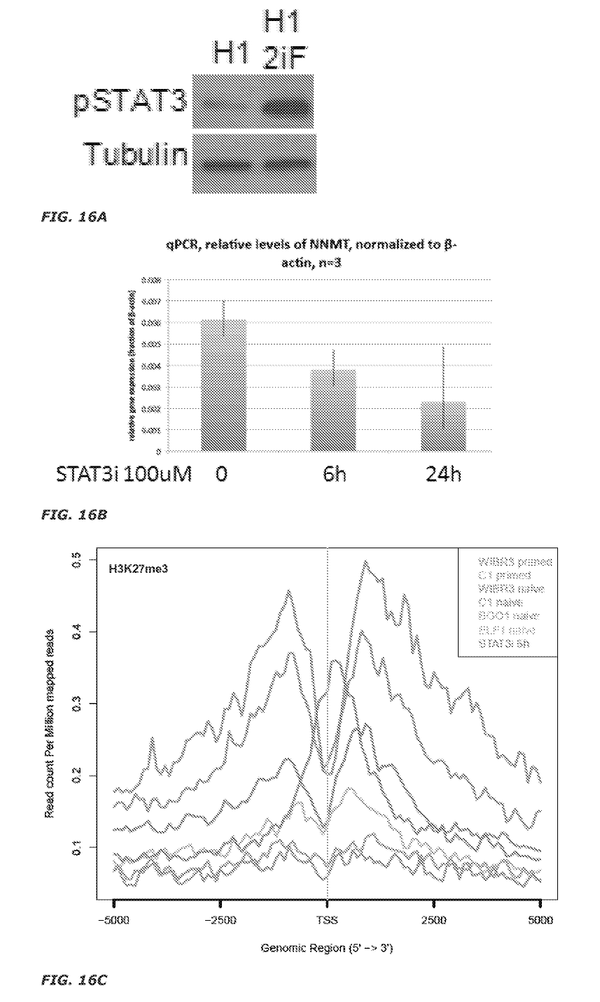

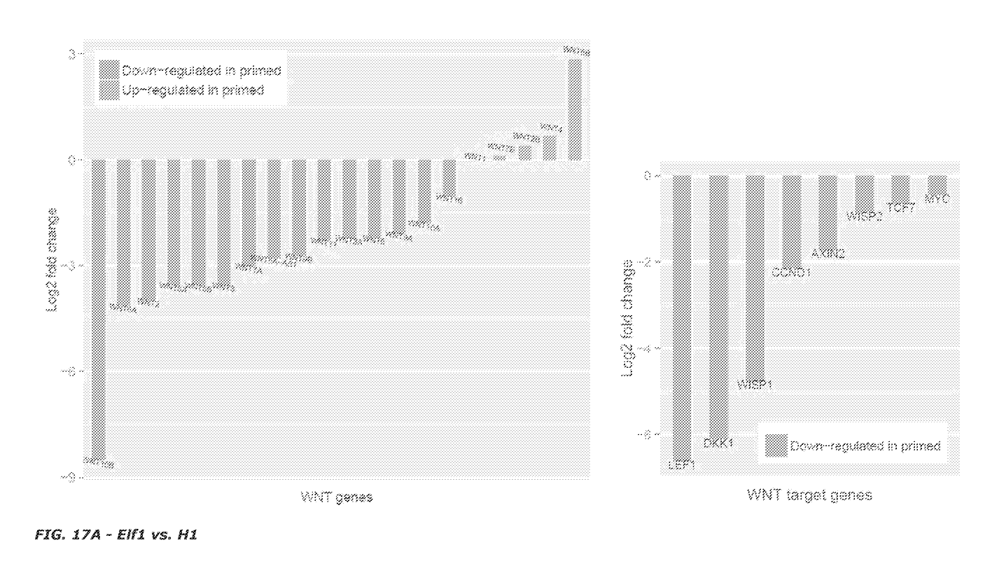

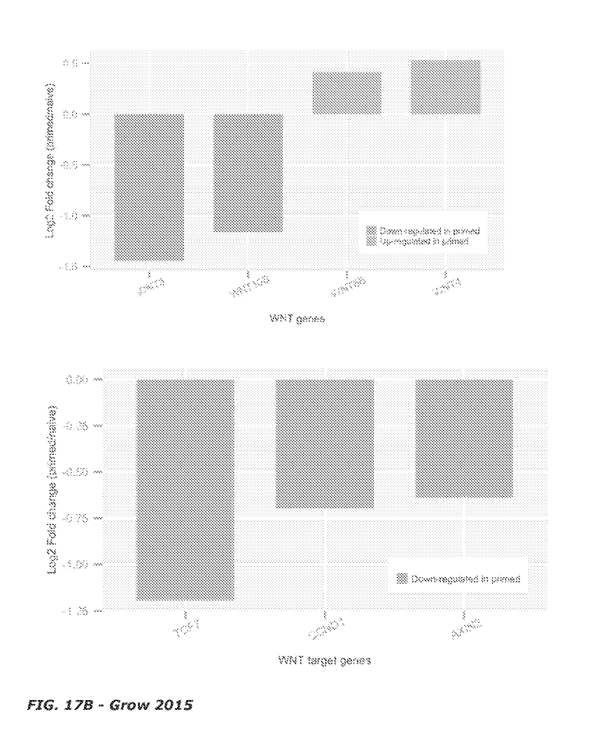

NNMT levels were also altered by inhibiting the LIF/STAT pathway. The LIF/STAT pathway was activated in naïve hESCs since they were grown in media supplemented with LIF. LIF is known to activate STAT3, which has been shown to bind to the NNMT promoter and activate its transcription. Interestingly, H1 toggled to more naïve state using 2iF8, which also have high level of NNMT (FIG. 4H) activates endogenous LIF pathway and show STAT3 phosphorylation (FIG. 16A). Treating naïve hESCs with a STAT3 inhibitor might affect NNMT expression and the repressive histone methylation pattern of those cells. qPCR analysis showed a reduction of NNMT expression on Elf1 cells as early as 6 hr after STAT3 inhibitor addition (FIG. 16B). Importantly, reduction of NNMT in naïve hESCs by STAT3 inhibitor also increased H3K27me3 and H3K9me3 marks, as shown by Western blot analysis (FIG. 5F). H3K27me3 in naïve hESCs was characterized by ChIP-seq analysis and a significant increase in H3K27me3 marks were observed at promoters after 6 hr STAT3 inhibitor treatment (FIG. 5G; FIG. 16C). Interestingly, over 25% of genes (313 genes) with primed-enriched H3K27me3 marks already showed increased H3K27me3 marks after 6 hr STAT3 inhibitor treatment (FIG. 5H). Windowed chromatin heatmaps revealed a dramatic increase in H3K27me3 marks close to the transcription start site of these 313 genes in the naïve to primed transition as well as after 6 hr STAT3 inhibitor treatment (FIG. 5I).

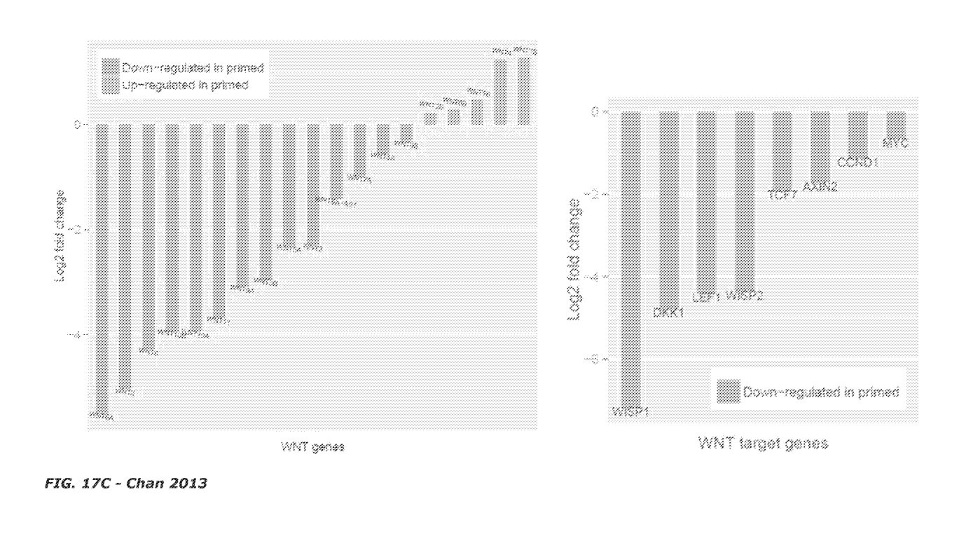

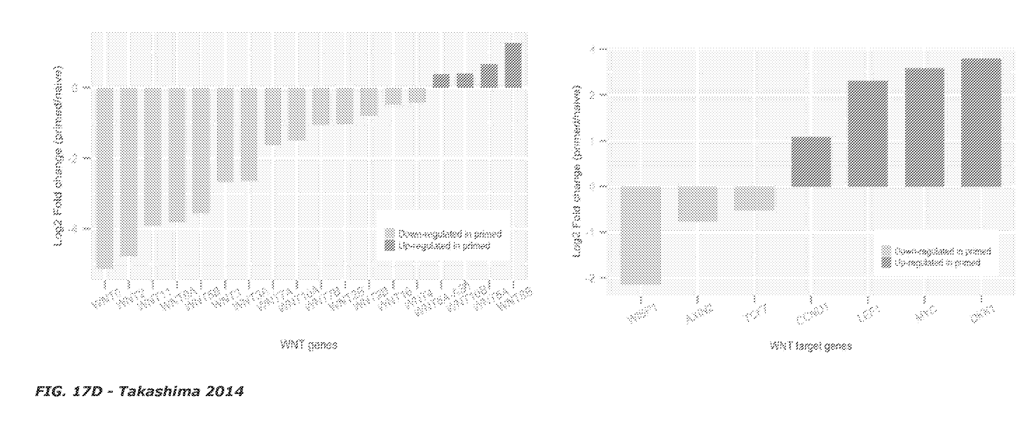

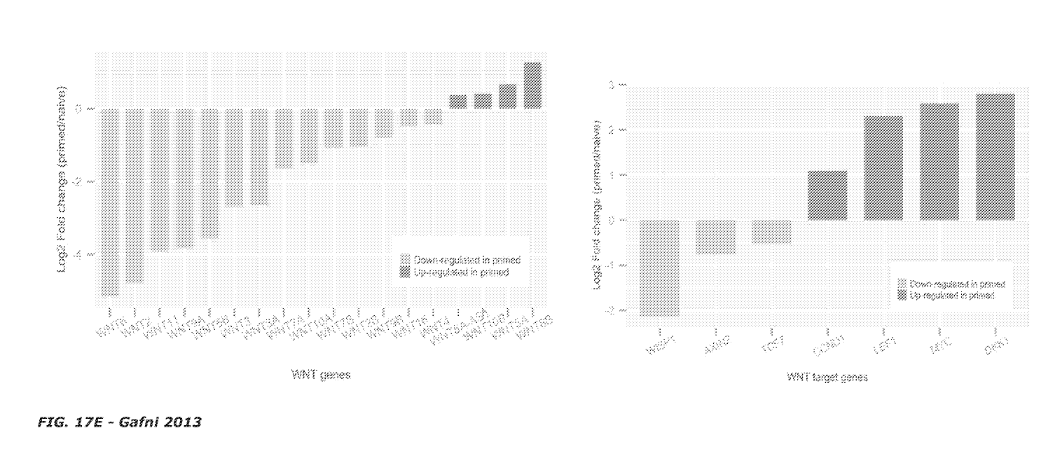

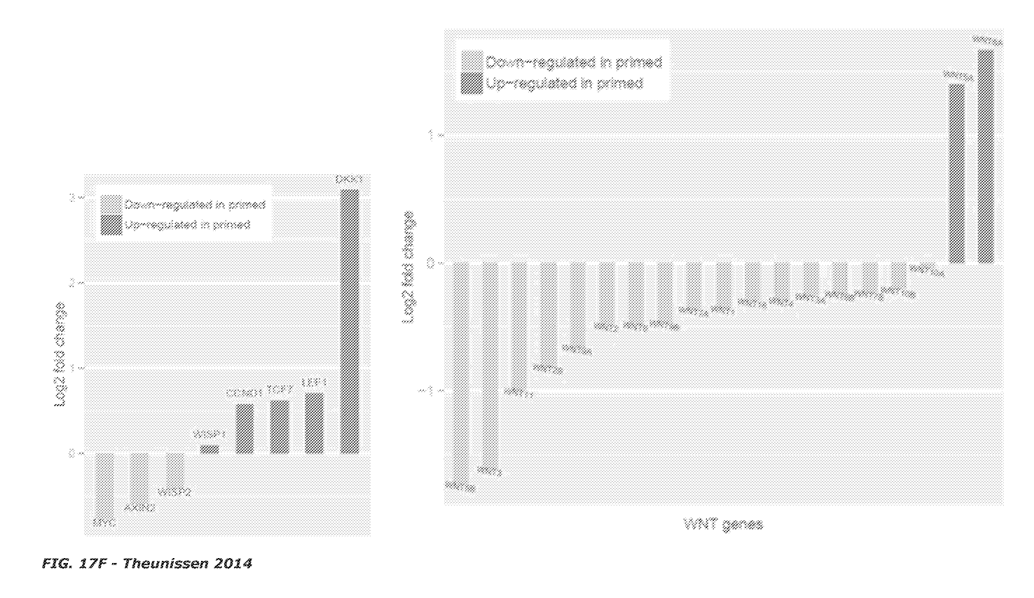

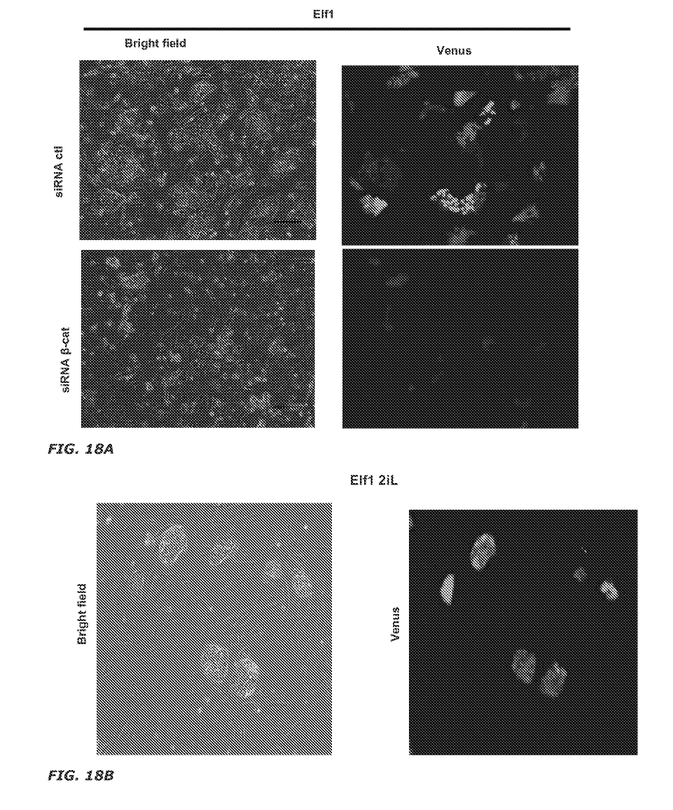

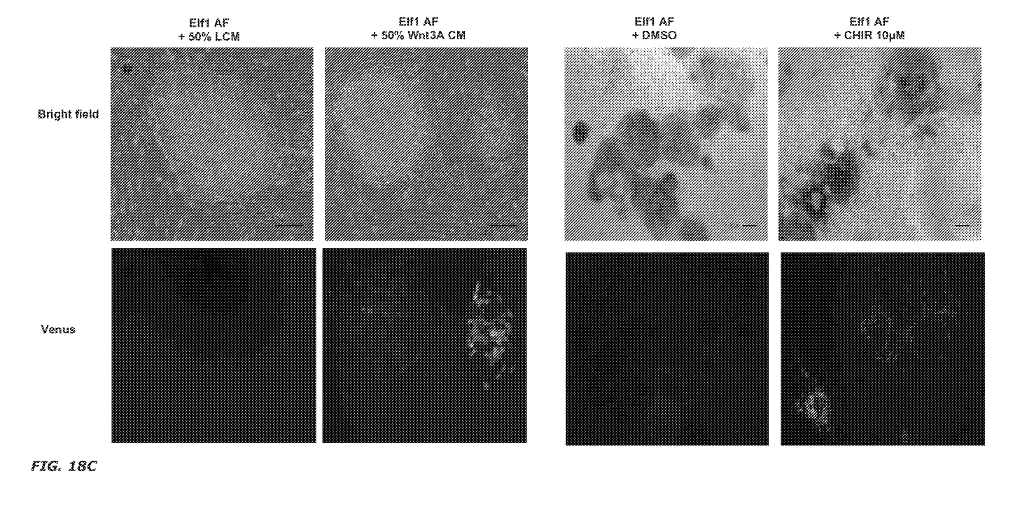

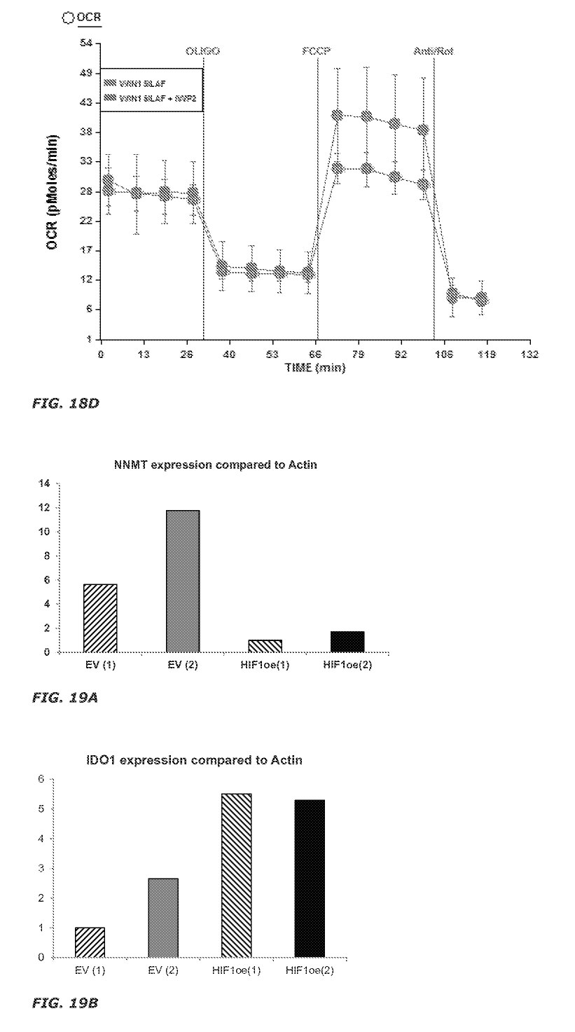

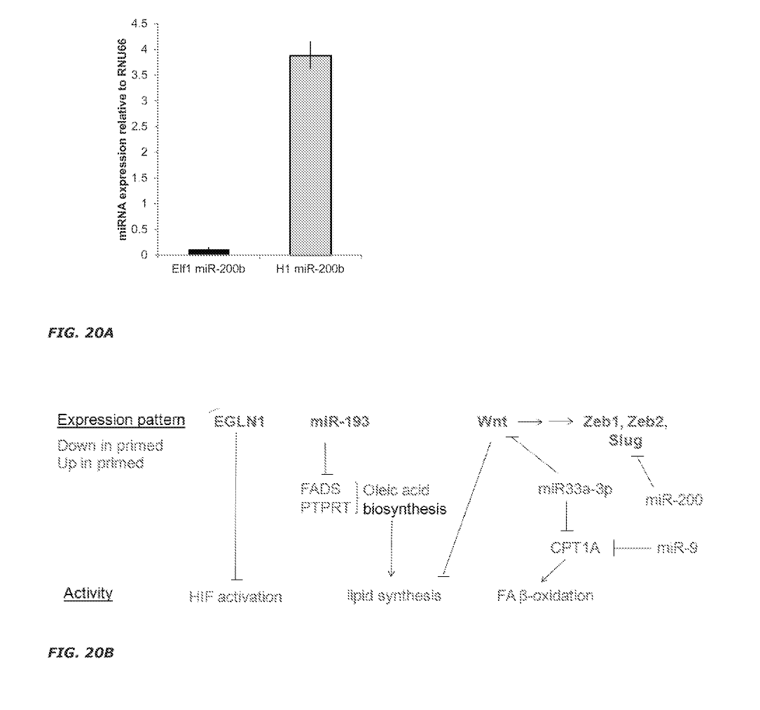

To determine the functional consequence of H3K27me3 marks after STAT3 inhibitor treatment, the overlapping 313 genes were analyzed through RNA-seq and ChIP-seq data. The majority of these genes are developmentally regulated genes that are repressed in primed compared to naïve state. Importantly, among the 313 genes with repressive H3K27me3 marks are Wnt ligands Wnt5 and Wnt9, and Wnt targets, ZEB1, ZEB2 and SLUG. Further, the majority of Wnt ligands and target genes are downregulated in primed compared to naïve hESCs, suggesting that the Wnt pathway might be inactivated during the naïve to primed transition (FIG. 17A-17F, FIG. 1G, Vime). The Wnt pathway may additionally be downregulated by the primed state enriched miRNAs, miR-33a and miR-200b (FIG. 3F; FIG. 20A), which are predicted regulators of Wnt and Zeb respectively (Targetscan). In addition, miR-155-5p, miR-148-3p, and miR-130a-3p which have been shown to target JARID2 were all upregulated in naïve compared to primed state hESCs, consistent with the observed upregulation of JARID2 protein in primed hESCs (FIG. 1L-1M). Previous studies have revealed that in human and mouse primed ESCs the Wnt pathway is not active and forced activation of the pathway leads to differentiation. The activity of the Wnt pathway in naïve hESCs revealed that while a Wnt pathway activity reporter is not activated in primed hESCs, strong activation is observed in naïve hESCs (FIG. 5J; FIG. 18A-18D). Wnt activity in naïve hESCs is dependent on β-catenin since siRNA (β-cat) or XAV939 treatment dramatically downregulated the reporter activity (FIG. 5K, FIG. 18A). In addition, the Wnt ligand is produced by the naïve hESCs since IWP2, an inhibitor that represses Wnt palmitylation also represses the reporter activity in naïve cells (FIG. 5K). Inhibition of Wnt in naïve hESCs reduces expression of NNMT and naïve hESC enriched microRNA miR-3728 and accelerates the transition toward the primed metabolic state (FIG. 5L-5N, FIG. 18D). These data reveal that the robust Wnt activity in naïve hESC is among the earliest responders to the repressive H3K27me3 marks during naïve to primed hESC transition.

Another gene category of interest in the group of 313 early H3K27me3 responders is metabolic genes. A dramatic increase of H3K27me3 marks was observed in prolyl hydroxylase 2 (EGLN1), ECHS1, HIGD1 and miR-193 promoters in the primed state as well as in STAT3 inhibitor treated Elf1 cells, compared to the naïve state. The repressive marks in these promoters correlated with the observed reduced gene expression in the primed state. Since EGLN1 induces HIF1 degradation, its repression in the primed state (FIG. 5O) suggests that HIF1 is stabilized (FIG. 5P). Analysis of HIF1 protein in the naïve and primed states supports this hypothesis (FIG. 1J). Furthermore, HIF1 stabilization accelerates primed hESC markers, including increased H3K27me3 marks (FIG. 5Q, FIG. 19A-19B). miR-193 is predicted to target enzymes involved in oleic acid biosynthesis (FADS and PTPRT, TargetScan; Mirtarbase). Metabolomic analysis showed that oleic acid levels are increased in primed hESCs compared to naïve, suggestive of increased fatty acid synthesis (FIG. 20B). Furthermore, repressive epigenetic marks and reduced expression of ECHS1 and HIGD1A correlate with reduced beta-oxidation and electron transport chain activity in primed hESCs (FIG. 1I, FIG. 3J).

In summary, downregulated NNMT expression results in increased levels of SAM and induction of H3K27me3 repressive marks. JARID2 protein upregulation in primed state may give further regulation/target specificity for the H3K27me3 marks generated by PRC2 (FIG. 1L-1M) since JARID2, the catalytically inactive demethylase is an essential component of PRC2 in ESC. NNMT downregulation activates HIF, represses Wnt pathway and the electron transport chain supercomplex regulator HIGD1, thereby inducing the metabolic switch with a dramatic reduction of mitochondrial activity, suggesting that NNMT reduction moves the cells towards the primed state (FIG. 5N, 5R).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of detecting the developmental state of a stem cell, comprising:
measuring the levels of one or more metabolites in a culture of the stem cells; and
determining whether the stem cells are in a naïve or a primed state,
wherein naïve state stem cells have higher levels of the one or more naïve state metabolites than the primed state stem cells, and primed state stem cells have higher levels of one or more primed state metabolites than the naïve state stem cells.

2. The method of claim 1, wherein the one or more metabolites are primed state metabolites or naïve state metabolites.

3. The method of claim 1, wherein the stem cell is an embryonic stem cell, a germline stem cell, an induced pluripotent stem cell, an adult stem cell, a hematopoietic stem cell, or a dental pulp stem cell.

4. The method of claim 1, where the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

5. The method of claim 1, wherein the primed state metabolite is a breakdown product of tryptophan, S-adenosyl methionine (SAM), succinate, fructose (1,6/2,6)-biphosphonate, lactate, methionine, nicotinamide, kynurenine, long carbon chain lipids, or an aryl hydrocarbon receptor (AHR) ligand, or an inducer of indoleamine 2,3-diozygenase 1 (IDO1), IDO2, or tryptophan 2,3-dioxygenase 2 (TDO2), or an inhibitor of nicotinamide-N-methyl-transferase (NNMT).

6. The method of claim 1, wherein the naïve state metabolite is a glycogen synthase kinase 3 (GSK3) inhibitor, a mitogen-activated protein kinase (MEK) inhibitor, 1-methylnicotinamide (1-MNA), tryptophan, S-adenosylhomocysteine (SAH), or an inhibitor of indoleamine 2,3-diozygenase 1 (IDO1), IDO2, or tryptophan 2,3-dioxygenase 2 (TDO2), or an inducer of nicotinamide-N-methyl-transferase (NNMT).

7. The method of claim 5, wherein the AHR ligand is a halogenated aromatic hydrocarbon such as a polychlorinated dibenzodioxin (2,3,7,8-tetrachlorodibenzo-p-dioxin (TCDD), a dibenzofuran, or a biphenyl, a polycyclic aromatic hydrocarbon such as (3-methylcholanthrene, a benzo(a)pyrene, a benzanthracene, or a benzoflavone, a derivative of tryptophan such as an indigo dye or indirubin, a tetrapyrrole such as bilirubin, an arachidonic acid metabolite such as lipoxin A4 or prostaglandin G, a modified low-density lipoprotein, or a dietary carotenoid.

8. The method of claim 5, wherein the long carbon chain lipid has a carbon chain length between about 25 and about 50 carbons.

9. (canceled)

10. The method of claim 8, wherein the long carbon chain lipid has a carbon chain length longer than about 40 carbons.

11. The method of claim 6, wherein the MEK inhibitor is trametinib,
selumetinib, binimetinib, PD-325901, cobimetinib, CI1040, or PD035901

12. A method of promoting the transition of stem cells from one state to another state; wherein
(a) if the transition is from a naïve state to a primed state, the method comprises culturing the stem cell in a culture medium supplemented with at least one primed state metabolite; or
(b) if the transition is from a primed state to a naïve state, the method comprises culturing the stem cell in a culture medium supplemented with at least one naïve state metabolite; and
determining the level of kynurenine in the stem cells, wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

13. (canceled)

14. The method of claim 12, wherein the stem cell is an embryonic stem cell, a germline stem cell, an induced pluripotent stem cell, an adult stem cell, a hematopoietic stem cell, or a dental pulp stem cell.

15. (canceled)

16. The method of claim 12, wherein the stem cells are cultured with the primed state metabolite or the naïve state metabolite for at least three days to reach the desired state.

17.-23. (canceled)

24. A method of maintaining stem cells in a defined state, wherein;
(a) if the state is a naïve state, the method comprises culturing naïve state stem cells in a culture medium supplemented with at least one naïve state metabolite; or
(b) if the state is a primed state, the method comprises culturing primed state stem cells in a culture medium supplemented with at least one primed state metabolite.

25. (canceled)

26. The method of claim 25, wherein the stem cell is an embryonic stem cell, a germline stem cell, an induced pluripotent stem cell, an adult stem cell, a hematopoietic stem cell, and a dental pulp stem cell.

27. The method of claim 25, where the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

28. The method of claim 25, wherein the stem cells are cultured with the primed state metabolite or the naïve state metabolite for at least three days to reach the desired state.

29.-35. (canceled)

36. A method of inhibiting the transition of stem cells from one state to another, wherein;
(i) if inhibiting the transition from a primed state to a naïve state, the method comprises culturing primed state stem cells in a culture medium supplemented with at least one primed state metabolite; or
(ii) if inhibiting the transition from a naïve state to a primed state, the method comprises culturing naïve state stem cells in a culture medium containing at least one naïve state metabolite.

37. (canceled)

38. (canceled)

39. The method of claim 36, where the method further comprises determining the concentration of kynurenine and tryptophan in the culture media of the stem cells, wherein the culture media has not been supplemented with kynurenine or tryptophan, and wherein a kynurenine/tryptophan ratio lower than about 0.015 is indicative of a preponderance of naïve state stem cells and a kynurenine/tryptophan ratio higher than about 0.015 is indicative of a preponderance of primed state stem cells.

40.-51. (canceled)
